(19) United States
(12) Patent Application Publication
(10) Pub. No.: US 2026/0022192 A1
(43) Pub. Date: Jan. 22, 2026

(54) RECOMBINANT OLIGOSACCHARYLTRANSFERASES AND METHODS OF USE THEREOF

(71) Applicant: CORNELL UNIVERSITY, Ithaca, NY (US)

(72) Inventors: Matthew DELISA, Ithaca, NY (US); Myriam Belen SOTOMAYOR BURNEO, Painted Post, NY (US); May TAW, Wayne, PA (US)

(21) Appl. No.: 19/276,530

(22) Filed: Jul. 22, 2025

Related U.S. Application Data
(60) Provisional application No. 63/674,186, filed on Jul. 22, 2024.

(51) Int. Cl. C07K 16/32; C12N 9/10

(52) U.S. Cl. CPC C07K 16/32; C12N 9/1051; C07K 2317/24; C07K 2317/41; C07K 2317/52; C07K 2317/622

(57) ABSTRACT

The present disclosure is directed to a recombinant oligosaccharyltransferase (OST) capable of catalyzing the transfer of a glycan onto a sequon comprising an N−X−T motif, wherein X can be any amino acid. Also disclosed are nucleic acid sequences and vectors encoding the recombinant OST, as well as host cells comprising the recombinant OST, nucleic acid sequences, or vectors as described herein. The present disclosure is also directed to glycoproteins produced by the disclosed host cells, methods of producing glycosylated proteins, and systems comprising a plasmid encoding the recombinant OST.

This application claims the benefit of U.S. Provisional Patent Application Ser. No. 63/674,186, filed Jul. 22, 2024, which is hereby incorporated by reference in its entirety.

This invention was made with government support under W911NF-23-2-0039 awarded by the Defense Advanced Research Projects Agency. The government has certain rights in the invention.

The Sequence Listing is being submitted electronically in XML format and is hereby incorporated by reference in its entirety. Said XML copy, created on Jul. 21, 2025, is named 147402.009491.xml and is 203,220 bytes in size. No new matter is being introduced.

### FIELD

The present disclosure relates to recombinant oligosaccharyltransferases and methods of use thereof.

### BACKGROUND

Protein glycosylation is an important post-translational modification that occurs in all domains of life (Abu-Qarn et al., “Not Just for Eukarya Anymore: Protein Glycosylation in Bacteria and Archaea,” Curr. Opin. Struct. Biol. 18:544-550 (2008)). It is estimated that over half of all naturally occurring proteins in eukaryotes are glycoproteins (Apweiler et al., “On the Frequency of Protein Glycosylation, as Deduced from Analysis of the SWISS-PROT database,” Biochim. Biophys. Acta. 1473:4-8 (1999); Stanley et al. in Essentials of Glycobiology, Edn. 4th. (eds. A. Varki et al.) 103-116 (Cold Spring Harbor (NY); 2022); and Khoury et al., “Proteome-Wide Post-Translational Modification Statistics: Frequency Analysis and Curation of the Swiss-Prot Database,” Sci. Rep. 1 (2011), with an even greater proportion among therapeutic proteins (Seeberger et al. in Essentials of Glycobiology, Edn. 4th. (eds. A. Varki et al.) 771-784 (Cold Spring Harbor (NY); 2022). Of the different types of protein glycosylation, asparagine-linked (N-linked) glycosylation is the most common (Khoury et al., “Proteome-Wide Post-Translational Modification Statistics: Frequency Analysis and Curation of the Swiss-Prot Database,” Sci. Rep. 1 (2011) and Walsh et al., “Protein Posttranslational Modifications: The Chemistry of Proteome Diversifications,” Angew Chem. Int. Ed. Engl. 44:7342-7372 (2005)).

The central reaction in the pathway is catalyzed by the oligosaccharyltransferase (OST), which transfers a preassembled oligosaccharide from a lipid-linked oligosaccharide (LLO) donor to an asparagine residue within a consensus acceptor site or sequon (typically N−X−S/T where X≠P) in a newly synthesized protein (Shrimal et al., “Cotranslational and Posttranslocational N-Glycosylation of Proteins in the Endoplasmic Reticulum,” Semin Cell Dev Biol 41:71-78 (2015)). While N-linked glycosylation in eukaryotes, archaea, and bacteria share many mechanistic features, some notable differences have been observed, especially with respect to the OSTs that are central to these systems (Abu-Qarn et al., “Not Just for Eukarya Anymore: Protein Glycosylation in Bacteria and Archaea,” Curr. Opin. Struct. Biol. 18:544-550 (2008); Weerapana et al., “Asparagine-Linked Protein Glycosylation: From Eukaryotic to Prokaryotic Systems,” Glycobiology 16: 91R-101R (2006); and Dell et al., “Similarities and Differences in the Glycosylation Mechanisms in Prokaryotes and Eukaryotes,” Int. J. Microbiol. 2010:148178 (2010)). For example, most eukaryotic OSTs are hetero-octameric complexes comprised of multiple non-catalytic subunits and a catalytic subunit, STT3 (Kelleher & Gilmore, “An Evolving View of the Eukaryotic Oligosaccharyltransferase,” Glycobiology 16:47R-62R (2006); Mohanty, S. et al., “Structural Insight into the Mechanism of N-Linked Glycosylation by Oligosaccharyltransferase,” Biomolecules 10 (2020); Ramirez et al., “Cryo-Electron Microscopy Structures of Human Oligosaccharyltransferase Complexes OST-A and OST-B,” Science 366:1372-1375 (2019); and Wild et al., “Structure of the Yeast Oligosaccharyltransferase Complex Gives Insight into Eukaryotic N-Glycosylation,” Science 359:545-550 (2018)). In contrast, archaea and bacteria possess single-subunit OSTs (ssOSTs) that are homologous to STT3 (Mohanty, S. et al., “Structural Insight into the Mechanism of N-Linked Glycosylation by Oligosaccharyltransferase,” Biomolecules 10 (2020); Matsumoto et al., “Crystal Structures of an Archaeal Oligosaccharyltransferase Provide Insights Into The Catalytic Cycle Of N-Linked Protein Glycosylation,” Proc. Natl. Acad. Sci. USA 110:17868-17873 (2013); and Lizak et al., “X-Ray Structure of a Bacterial Oligosaccharyltransferase,” Nature 474(7351):350-355 (2011)). Another difference among the various OSTs is their distinct but overlapping acceptor sequon preferences. The prototypical bacterial ssOST, namely PglB from Campylobacter jejuni (CjPglB), recognizes a more stringent D/E−X−1−N−X+1−S/T (X−1, +1≠P) sequon compared to the N−XS/T sequon recognized by eukaryotic and archaeal OSTs (Kowarik et al., “Definition of the Bacterial N-Glycosylation Site Consensus Sequence,” EMBO J. 25(9):1957-1966 (2006)). However, this requirement for an acidic residue in the −2 position of the sequon, known as the “minus two rule”, is not universally followed by all bacterial ssOSTs. Indeed, several PglB homologs from the Desulfobacterota (formerly Deltaproteobacteria) phylum including D. alaskensis G20 (formerly D. desulfuricans G20) PglB (DaPglB), D. gigas DSM 1382 PglB (DgPglB), and D. vulgaris Hildenborough PglB (DvPglB) exhibit sequon specificities that are relaxed compared to CjPglB and overlap with those of eukaryotic and archaeal OSTs (Ollis et al., “Substitute Sweeteners: Diverse Bacterial Oligosaccharyltransferases with Unique N-Glycosylation Site Preferences,” Sci. Rep. 5:15237 (2015)).

To date, these and other functional details about bacterial ssOSTs come from studies where the C. jejuni protein glycosylation machinery has been functionally reconstituted in laboratory strains of Escherichia coli, a feat that was first demonstrated more than 20 years ago (Wacker et al., “N-Linked Glycosylation in Campylobacter Jejuni and its Functional Transfer into E. Coli,” Science 298:1790-1793 (2002)). Since that time, many groups have leveraged CjPglB and its homologs for performing N-linked glycosylation of diverse protein substrates. Included among these substrates are fragments of human immunoglobulin (IgG) such as CH2 or CH2-CH3 (hereafter fragment crystallizable (Fc) domain), which hold promise in the treatment of autoimmune disorders (Anthony et al., “Recapitulation of IVIG Anti-Inflammatory Activity with a Recombinant IgG Fc,” Science 320:373-376 (2008) and Debre et al., “Infusion of Fc Gamma Fragments for Treatment of Children with Acute Immune Thrombocytopeniaurpura,” Lancet 342:945-949 (1993)). However, the use of engineered E. coli for producing glycosylated Fc domains has been limited to the attachment of non-human glycan structures at mutated acceptor sequons (Ollis et al., “Substitute Sweeteners: Diverse Bacterial Oligosaccharyltransferases with Unique N-Glycosylation Site Preferences,” Sci. Rep. 5:15237 (2015); Fisher et al., “Production of Secretory and Extracellular N-Linked Glycoproteins in Escherichia Coli,” Appl. Environ. Microbiol. 77(3):871-881 (2011); Schwarz et al., “A Combined Method for Producing Homogeneous Glycoproteins with Eukaryotic N-Glycosylation,” Nat. Chem. Biol. 6(4):264-266 (2010); Schwarz et al., “Relaxed Acceptor Site Specificity of Bacterial Oligosaccharyltransferase In Vivo,” Glycobiology 21(1):45-54 (2011); and Valderrama-Rincon et al., “An Engineered Eukaryotic Protein Glycosylation Pathway in Escherichia Coli,” Nat. Chem. Biol. 8(5):434-436 (2012)). While some progress has been made to overcome these shortcomings, the overall poor glycosylation efficiency of Fc domains in E. coli (<5%) remains an unsolved problem that has discouraged efforts to develop this user-friendly host for biosynthesis of Fc domains, as well as their parental IgG counterparts, with relevant glycosylation.

The present disclosure is directed at overcoming these and other deficiencies in the art.

### SUMMARY

One aspect of the present disclosure is directed to a recombinant oligosaccharyltransferase (OST) capable of catalyzing the transfer of a glycan onto a sequon comprising an N−X−T motif, wherein X can be any amino acid.

Another aspect of the present disclosure is directed to a nucleic acid molecule encoding a recombinant oligosaccharyltransferase according to the present disclosure.

Another aspect of the present disclosure is directed to a vector comprising a nucleic acid sequence encoding a recombinant oligosaccharyltransferase according to the present disclosure and a promoter heterologous to the nucleic acid sequence encoding the recombinant oligosaccharyltransferase.

Another aspect of the present disclosure is directed to a host cell comprising a recombinant oligosaccharyltransferase, nucleic acid sequence, or vector according to the present disclosure.

Another aspect of the present disclosure is directed to a glycoprotein produced by the host cell according to the present disclosure.

A further aspect of the present disclosure is directed to a method of producing a glycosylated protein. This method involves providing a prokaryotic host cell expressing a heterologous prokaryotic oligosaccharyltransferase enzyme capable of transferring a glycan to an N-glycosylation acceptor site of a protein, said acceptor site comprising an N−X−T motif, where X can be any amino acid but proline, and culturing the prokaryotic host cell under conditions effective to produce a glycosylated protein.

Yet another aspect of the present disclosure is directed to a system comprising: a first plasmid encoding enzymes for N-glycan biosynthesis; a second plasmid encoding a recombinant oligosaccharyltransferase (OST) according to the present disclosure; and/or a third plasmid encoding a protein of interest.

### BRIEF DESCRIPTION OF THE DRAWINGS

FIGS. 1A-1C demonstrate bioprospecting of Desulfovibrio species for functional PglB homologs. FIG. 1A is a phylogenetic tree of the PglB homologs evaluated in the present disclosure. The curated list of enzymes was generated from a BLAST search using DaPglB and DgPglB as the query sequences. CjPglB and ClPglB were added for comparison. The tree was generated by the neighbor-joining method from multiple sequence alignment using Molecular Evolutionary Genetics Analysis version 11 (MEGA11) software (Tamura et al., “MEGA11: Molecular Evolutionary Genetics Analysis Version 11,” Mol. Biol. Evol. 38:3022-3027 (2021), which is hereby incorporated by reference in its entirety). FIG. 1B provides immunoblot analysis of periplasmic fractions from CLM24 cells transformed with plasmid pMW07-pglΔBCDEF encoding genes for biosynthesis of a modified C. jejuni heptasaccharide glycan (GalNAc₅(Glc)GlcNAc), plasmid pBS-scFv13-R4^{DQNAT} encoding the scFv 13-R4^{DQNAT} acceptor protein, and a derivative of plasmid pMLBAD encoding one of the PglB homologs as indicated. The first two lanes in left and right panels of FIG. 1A and FIG. 1C were loaded with the same positive and negative control samples (marked with asterisk). Blots were probed with polyhistidine epitope tag-specific antibody (anti-His) to detect the C-terminal 6x-His tag on the acceptor protein (top panel) and hR6 serum specific for the C. jejuni heptasaccharide glycan (bottom panel). Molecular weight (M_{W}) markers are indicated on the left. The g0 and g1 arrows indicate unglycosylated and monoglycosylated acceptor proteins, respectively. Blots are representative of biological replicates (n=3). FIG. 1C are bar graphs showing glycosylation efficiency determined by densitometric analysis as described in the methods, with data reported as mean±SD. Red bars in a color version of this image correspond to positive and negative controls generated with CjPglB and CjPglB^{mut}, respectively; blue bars in a color version of this image correspond to samples generated with Desulfovibrio PglBs. Statistical significance was determined by unpaired two-tailed Student's t-test. Calculated p values are represented as follows: *, p<0.05; ***, p<0.001; ns, not significant.

FIGS. 2A-2D demonstrate the glycosylation of non-canonical sequons by Desulfovibrio PglB homologs. FIG. 2A provides immunoblot analysis of periplasmic fractions from CLM24 cells transformed with the following plasmids: pMW07-pglΔBCDEF for making GalNAc₅(Glc)GlcNAc; a derivative of pMLBAD encoding one of the PglB homologs as indicated; and pBS-scFv13-R4^{AQNAT} encoding the scFv13-R4(N34L/N77L) acceptor protein with AQNAT (SEQ ID NO: 22) sequon. Blots were probed with anti-His antibody (top panel) and hR6 serum (bottom panel). Molecular weight (M_{W}) markers are indicated on the left. The g₀ and g₁ arrows indicate unglycosylated and monoglycosylated acceptor proteins, respectively. Blots are representative of biological replicates (n=3). FIG. 2B are bar graphs showing glycosylation efficiency determined by densitometric analysis as described in the methods, with data reported as mean±SD. Red bars in a color version of this image correspond to positive and negative controls generated by CjPglB with scFv13-R4^{DQNAT} or scFv13-R4(N34L/N77L)^{AQNAT} as acceptors, respectively; blue bars in a color version of this image correspond to samples generated with Desulfovibrio PglBs. Statistical significance was determined by unpaired two-tailed Student's t-test. Calculated p values are represented as follows: *, p<0.05; ***, p<0.001; ns, not significant. FIG. 2C and FIG. 2D provide the results of immunoblot and densitometric analysis similar to FIG. 2A and FIG. 2B, but with plasmid pBS-scFv13-R4^{QYNST} encoding scFv13-R4(N34L/N77L) with QYNST (SEQ ID NO: 5) sequon. Red bars in a color version of this image correspond to positive and negative controls generated by CjPglB with scFv13-R4^{DQNAT} or scFv13-R4(N34L/N77L)^{QYNST} as acceptors, respectively; blue bars in a color version of this image correspond to samples generated with Desulfovibrio PglBs. The first two lanes in left and right panels of FIG. 2A and FIG. 2C were loaded with the same positive and negative control samples (marked with asterisk).

FIGS. 3A-3C demonstrate the molecular determinants of DmPglB acceptor-site specificity. FIG. 3A provides immunoblot analysis of periplasmic fractions from CLM24 cells transformed with the following plasmids: pMW07-pglΔBCDEF for making GalNAc₅(Glc)GlcNAc; pMLBAD encoding DmPglB, DmPglB^{mut}, CjPglB or CjPglB^{mut}; and pBS-scFv13-R4^{XQNAT} encoding the scFv13-R4 with each of the 20 amino acids in the −2 position of the C-terminal sequon as indicated. Blots were probed with anti-His antibody (top panel) and hR6 serum (bottom panel). Molecular weight (M_{W}) markers are indicated on the left. The g0 and g1 arrows indicate unglycosylated and monoglycosylated acceptor proteins, respectively. Blots are representative of biological replicates (n=3). FIG. 3B provides heatmap analysis of the relative −2 amino acid preference of CjPglB, DgPglB, and DmPglB. Relative preferences (weaker=white; stronger=dark cyan) were determined based on densitometry analysis of the glycosylation efficiency for each acceptor protein in the anti-His immunoblot (see FIGS. 10A-10B and FIG. 11 for efficiency data). FIG. 3C is a sequence logo showing experimentally determined acceptor-site specificity of DmPglB using glycoSNAP-based library screening of YebF(N24L)-Im7^{XXNXT}.

FIGS. 4A-4C demonstrate molecular determinants of relaxed acceptor-site specificity of DmPglB. FIG. 4A shows the electrostatic potential of various OST peptide-binding pockets modeled with either DQNAT (SEQ ID NO: 6) (top) or QYNST (SEQ ID NO: 5) (bottom) acceptor peptides (yellow in a color version of this image). Electrostatic surfaces were generated based on calculations using the adaptive Poisson-Boltzmann solver (APBS) (Baker et al., “Electrostatics of Nanosystems: Application to Microtubules and the Ribosome,” Proc. Natl. Acad. Sci. USA 98:10037-10041 (2001), which is hereby incorporated by reference in its entirety). FIG. 4B provides sequence alignments of conserved, short motifs in eukaryotic STT3s (human and plant STT3A and STT3B, protozoan Leishmania major STT3D and Trypanosoma brucei TbSTTA) and bacterial ssOSTs (ClPglB, CjPglB, DgPglB, DmPglB, DiPglB). Alignments shown were made using Clustal Omega web server multiple alignment editor (Madeira et al., “Search and Sequence Analysis Tools Services from EMBL-EBI in 2022,” Nucleic Acids Res 50:W276-W279 (2022), which is hereby incorporated by reference in its entirety). Conserved residues are shown in bold uppercase text and shaded gray in a color version of this image while notable residues that deviate between eukaryotic and bacterial sequences are shown in bold lowercase text and are shaded yellow in a color version of this image. FIG. 4C provides a structural model of QYNST (SEQ ID NO: 5) peptide (yellow in a color version of this image) in the peptide-binding pocket of the same OSTs in FIG. 4A. Depicted in green in a color version of this image are amino acids at the entrance to the peptide-binding cavity that cluster to create a positively charged patch in ClPglB but are neutral in all other OSTs. The SVSE (SEQ ID NO: 34)/SVIE (SEQ ID NO: 35)/TIXE (SEQ ID NO: 33) motifs are depicted in gold in a color version of this image.

FIGS. 5A-5C demonstrate glycosylation of the native QYNST (SEQ ID NO: 5) sequon in IgG Fc domains by DmPglB. FIG. 5A provides non-reducing immunoblot analysis of protein A-purified proteins from whole-cell lysate of CLM24 cells transformed with the following plasmids: pMW07-pglΔBCDEF for making GalNAc₅(Glc)GlcNAc (left) or pMW07-pglΔBICDEF for making GalNAc₅GlcNAc (right); pMLBAD encoding CjPglB, DgPglB, DmPglB, or DmPglB^{mut}; and pTrc99S-hinge-Fc encoding hinge-Fc derived from human IgG1. Blots were probed with anti-human IgG (anti-IgG) to detect human Fc (top panel) and hR6 serum (bottom panel). Molecular weight (M_{W}) markers are indicated on the left. The g0, g1, and g2 arrows indicate unglycosylated, monoglycosylated, and diglycosylated Fc proteins, respectively. Blots are representative of biological replicates (n=3). FIG. 5B are graphs showing glycosylation efficiency determined as above with data reported as the mean±SD. Statistical significance was determined by unpaired two-tailed Student's t-test. Calculated p values are represented as follows: ****, p<0.0001. FIG. 5C provides non-reducing immunoblot analysis similar to FIG. 5A but with JUDE-1 cells transformed with plasmid pMAZ360-YMF10-IgG encoding a full-length chimeric IgG1 specific for PA along with plasmids for glycan biosynthesis and ssOST as indicated. Asterisks denote band shifts due to glycosylation of HC-LC dimer.

FIGS. 6A-6C demonstrate chemoenzymatic remodeling of E. coli-derived hinge-Fc glycans. FIG. 6A is a schematic representation of the chemoenzymatic reaction for trimming and remodeling hinge-Fc glycans. FIG. 6B provides immunoblot analysis of the four E. coli-derived glycoforms (from left to right): aglycosylated hinge-Fc, glycosylated GalNAc₅GlcNAc-hinge-Fc, GlcNAc-hinge-Fc, and G2-hinge-Fc. Blot was probed with anti-human IgG (anti-IgG) to detect human Fc. Molecular weight (M_{W}) markers are indicated on the left. The g0, g1, and g1 arrows indicate unglcyosylated, monoglycosylated, and diglycosylated Fc proteins, respectively. Blot is representative of biological replicates (n=3). FIG. 6C describes ELISA analysis of the same constructs in FIG. 6B with FcγRIIIA-V158 as immobilized antigen. Data are average of biological replicates (n=3)±SD.

FIGS. 7A-7B demonstrate glycosylation of scFv 13-4^{DQNAT/QYNST} by Desulfovibrio PglBs. FIG. 7A and FIG. 7B show longer exposure of same immunoblots in left-hand panels of FIG. 1B and FIG. 2C, respectively, revealing six Desulfovibrio PglB homologs that are capable of glycosylating scFv13-R4^{DQNAT} (FIG. 7A) and four Desulfovibrio PglB homologs that are capable of glycosylating scFv13-R4(N34L/N77L)^{QYNST} (FIG. 7B).

FIG. 8 provides MS analysis of scFv13-R4^{QYNST} glycosylation mediated by DmPglB. Intact glycopeptide mass corresponding to ALEGGQYN (SEQ ID NO: 57)[+HcxNAc(6)Hex(1)] ST released by trypsin/α-lytic protease treatment of glycosylated scFv13-R4^{QYNST} and detected by LC-MS/MS. Site-specific glycan attachment was confirmed by glycopeptide feature and peptide backbone identification under HCD fragmentation.

FIGS. 9A-9C analyze DmPglB autoglycosylation. FIG. 9A shows immunoblot analysis of glycosylated DmPglB generated by incubating purified aglycosylated DmPglB in an IVG reaction containing LLOs bearing the GalNAc₅GlcNAc glycan. Blots were probed with polyhistidine epitope tag-specific antibody (anti-His) to detect the C-terminal 10x-His tag on DmPglB (left panel) and hR6 serum specific for the hexasaccharide glycan (right panel). Molecular weight (M_{W}) markers are indicated on the left. The asterisks indicate mono-, and diglycosylated DmPglB. Blots are representative of biological replicates (n=3). FIG. 9B provides a structural model of DmPglB generated using the AlphaFold2 protein structure prediction algorithm implemented with ColabFold. Unstructured C-terminus highlighted in red with two identified glycosylation sequons colored in blue and marked with heptasaccharide schematics. FIG. 9C provides the results of glycoprotcomics analysis identifying concurrent HexNAc(6) glycosylation on sites EANGT (SEQ ID NO: 24) and AANAT (SEQ ID NO: 25). The example HCD MS2 spectrum shows informative fragment ions localizing two simultaneous HexNAc(6) glycan attachments on sites EANGT (SEQ ID NO: 24) and AANAT (SEQ ID NO: 25), and a deamidation modification on site PTNGT (SEQ ID NO: 58) along the semi-specific 48 amino acid tryptic glycopeptide. The attempt to fully disentangle the 5 potential glycosylation sites by trypsin/α-lytic protease sequential digestion could not confidently localize HexNAc(6) glycan attachment on any of the other sites, namely PTNGT (SEQ ID NO: 58), GENTT (SEQ ID NO: 59), or PANTT (SEQ ID NO: 60), indicating low glycan occupancy on these sites. Taken together, mass spectrometry evidence supports the major autoglycosylation state of DmPglB as g2, specifically on sites EANGT (SEQ ID NO: 24) and AANAT (SEQ ID NO: 25).

FIGS. 10A-10B investigate the molecular determinants of DgPglB acceptor-site specificity. FIG. 10A shows immunoblot analysis of periplasmic fractions from CLM24 cells transformed with the following: plasmid pMW07-pglΔBICDEF, plasmid pMAF10 encoding DgPglB, CjPglB, or CjPglBmut, and plasmid pBS-scFv13-R4XQNAT encoding the scFv13-R4(N34L/N77L) acceptor protein with one of the 20 sequon variants at the C-terminus as indicated. Blots were probed with polyhistidine epitope tag-specific antibody (anti-His) to detect the C-terminal 6x-His tag on the acceptor protein (top panel) and hR6 serum specific for the C. jejuni heptasaccharide glycan (bottom panel). Molecular weight (M_{W}) markers are indicated on the left. The g0 and g1 arrows indicate un-and monoglycosylated acceptor proteins, respectively. Blots are representative of biological replicates (n=2). FIG. 10B is a bar graph showing glycosylation efficiency was determined by densitometric analysis as described in the methods, with data reported as mean±SD. Red bars correspond to positive and negative controls generated by CjPglB and CjPglBmut with scFv13-R4^{DQNAT} as acceptor; blue bars correspond to samples generated by DgPglB with each of the 20 scFv13-R4(N34L/N77L)^{XQNAT} variants as indicated. Statistical significance was determined by unpaired two-tailed Student's t-test. Calculated p values are represented as follows: *, p<0.05; **, p<0.01; ns, not significant.

FIG. 11 is a graph showing the molecular determinants of DmPglB acceptor-site specificity. Glycosylation efficiency corresponding to the glycoprotein samples in FIG. 3A immunoblot. Efficiency was determined by densitometric analysis as described in the methods, with data reported as mean±SD. Red bars correspond to positive control generated by CjPglB with scFv13-R4^{DQNAT} as acceptor and negative controls generated by CjPglBmut or DmPglBmut with scFv13-R4^{DQNAT} as acceptor; blue bars correspond to samples generated by DmPglB with each of the 20 scFv13-R4(N34L/N77L)^{XQNAT} variants as indicated.

FIG. 12 is a structural model of DmPglB that reveals key active site residues. Structural model of DmPglB was generated using the AlphaFold2 protein structure prediction algorithm implemented with ColabFold as described in the methods. Residues colored purple in a color version of this image indicate key active site amino acids D55N and E363Q that are in close proximity of the DQNAT (SEQ ID NO: 6) acceptor sequon peptide colored in yellow and correspond to active site residues D56 and E319 in ClPglB or D54N and E316Q in CjPglB. Double mutation of these residues renders the enzyme catalytically inactive (Lizak et al., “X-Ray Structure of a Bacterial Oligosaccharyltransferase,” Nature 474(7351):350-355 (2011) and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety).

FIGS. 13A-13D illustrate an unbiased determination of DmPglB sequon specificity using glycoSNAP. FIG. 13A shows immunoblot analysis of acceptor proteins in colony secretions derived from E. coli CLM24 carrying a plasmid encoding either YebF(N24L)-Im7^{DQNAT} along with plasmids encoding N-glycosylation machinery with either wild-type DmPglB (wt) or an inactive mutant (mut). Blots were probed with anti-polyhistidine antibody (anti-His; red in a color version of this image) to detect acceptor proteins and the lectin soybean agglutinin (SBA; green in a color version of this image) to detect glycans. Overlay of anti-His and SBA images is shown as merge. FIG. 13B shows GlycoSNAP screen of sequon library whereby colonies were replicated on nitrocellulose transfer membranes and membranes were probed with anti-His antibody (red in a color version of this image) and SBA lectin (green in a color version of this image) as in FIG. 13A. Merged image reveals positive hits (yellow colonies in a color version of this image) that are indicated by white arrows. FIG. 13C shows an immunoblot of periplasmic fractions from CLM24 cells transformed with the following: plasmid pMW07-pglΔBCDEF, plasmid pMLBAD encoding DmPglB or DmPglBmut, and plasmid pTrc99S-YebF(N24L)-Im7XXNXT encoding one of the sequon mutants at the C-terminus as indicated. Blots were probed with anti-His antibody to detect the C-terminal 6x-His tag on the acceptor protein (top panel) and hR6 serum specific for the C. jejuni heptasaccharide glycan (bottom panel). Molecular weight (M_{W}) markers are indicated on the left. The g0 and g1 arrows indicate unglycosylated and monoglycosylated acceptor proteins, respectively. Blots are presentative of biological replicates (n=3). FIG. 13D is a list of all 65 glycoSNAP hits isolated from XXNXT (SEQ ID NO: 39) sequon library in this study with multiply identified hits indicated in parentheses.

FIGS. 14A-14D demonstrate the quantitative in vitro determination of PglB catalysis. FIG. 14A shows tricine SDS-PAGE analysis of peptide glycosylation determined by quantification of fluorescently labeled substrate and product over time using 0.5 μM peptide substrate and 0.18 μM PglB. Before gel loading, samples were diluted such that the concentration of total peptide in each lane was identical. Glycosylated peptide (g1) is separated from non-glycosylated peptide (g0), and bands were visualized by a fluorescence gel scan at 488 nm excitation and 526 nm emission. FIG. 14B is a graph showing the determination of turnover rates from the reactions in FIG. 14A. The amount of glycopeptide was determined from band intensities of fluorescence gel scans. The sum of the signals for glycosylated and non-glycosylated peptide for each lane was defined as 100%. Data were fitted by linear regression and the turnover rate was calculated from the slope of the fit (from top to bottom, R2=0.9229, 0.9705, 0.9424). FIG. 14C shows tricine SDS-PAGE analysis of in vitro glycosylation with different amounts of fluorescently labeled peptide indicated above the lanes and 0.18 μM PglB. Separation of glycopeptides and gel scanning was performed as in FIG. 14A. FIG. 14D is a graph showing the determination of Michaelis-Menten kinetics for the in vitro glycosylation reactions shown in FIG. 14C with quantification of glycosylated peptide performed as in FIG. 14A. Data were fitted by nonlinear regression according to the Michaelis-Menten formula using Prism 10 for MacOS version 10.3.0 (from left to right, R2=0.9462, 0.8981, 0.8243). Each data point in FIG. 14B and FIG. 14D represents the average of three individual reactions±SD.

FIGS. 15A-15B illustrate conserved sequence motifs in eukaryotic and bacterial OSTs. FIG. 15A is a schematic of PglB topological structure showing positions of the short motifs that are highly conserved in eukaryotic and bacterial OSTs. FIG. 15B provides amino acid sequences corresponding to the following motifs: SVSE (SEQ ID NO: 34)/TIXE (SEQ ID NO: 33), WWDXG (SEQ ID NO: 23), DNXTZNX (T/S) (SEQ ID NO: 62)/DGGK (SEQ ID NO: 32), and DK/MI. SVSE (SEQ ID NO: 34)/TIXE (SEQ ID NO: 33) motif occurs in EL5, with SVSE (SEQ ID NO: 34) present in eukaryotes and TIXE (SEQ ID NO: 33) present in bacteria (Taguchi et al., “The Structure of an Archaeal Oligosaccharyltransferase Provides Insight into the Strict Exclusion of Proline from the N-Glycosylation Sequon,” Commun. Biol. 4(1):941 (2021), which is hereby incorporated by reference in its entirety); Desulfovibrio sp. PglBs possess eukaryotic-like SVIE (SEQ ID NO: 35)/SIIE (SEQ ID NO: 36) motif. DGGK (SEQ ID NO: 32) motif is conserved among Campylobacter PglBs (Barre et al., “A Conserved DGGK Motif is Essential for the Function of the PglB Oligosaccharyltransferase from Campylobacter jejuni,” Glycobiology 27(10):978-989(2017), which is hereby incorporated by reference in its entirety); all Desulfovibrio sp. PglBs possess (D/N) G (G/A/S) (H/N/Q/R/S) (SEQ ID NO: 61) motif. Eukaryotic STT3s possess double sequon motif, DNXTZNX (T/S) (SEQ ID NO: 62) (where X and Z=any residue), in this location. WWDXG (SEQ ID NO: 23) (where X=Y or W) and DK/MI motifs (Igura et al. EMBO J 2008) occur in C-terminal globular domain, where eukaryotic DK motif=DXXKXXX (M/I) (SEQ ID NO: 37) and bacterial MI motif=MXXIXXX (I/V/W) (SEQ ID NO: 38); Desulfovibrio sp. PglBs possess a hybrid XL motif, (D/M/L) XXLXXXI (SEQ ID NO: 63). Asterisk denotes location corresponding to conserved R331 residue in ClPglB that provides salt bridge to −2 residue in bound DQNATF (SEQ ID NO: 64) substrate peptide (Lizak et al., “X-Ray Structure of a Bacterial Oligosaccharyltransferase,” Nature 474(7351):350-355 (2011), which is hereby incorporated by reference in its entirety).

FIGS. 16A-16C provide MS analysis of Fc glycosylation mediated by DmPglB. Intact glycopeptide masses corresponding to EEQYN[+Glycan] STYR were detected from the following samples: GalNAc₅(Glc)GlcNAc-hinge-Fc (FIG. 16A); GalNAc₅(Glc)GlcNAc-IgG (FIG. 16B); and GalNAc₅GlcNAc-IgG (FIG. 16C). Glycan attachment was confirmed based on canonical oxonium ions (HexNAc, HexNAcHex, etc.) and neutral losses (Pep, Pep+HexNAc, etc.) generated by N-glycopeptides under HCD fragmentation. Peptide backbone identities were confirmed based on b/y fragments, when limited backbone fragmentation was available (panel b), confident identification was made based on accurate precursor mass of the MS2 scan and its alignment to the retention time of non-glycosylated peptide EEQYNSTYR (SEQ ID NO: 40) in the same chromatogram.

FIGS. 17A-17B illustrate remodeling bacteria-derived IgG1-Fc with eukaryotic N-glycans. FIG. 17A shows LC-ESI-MS monitoring of protein A-purified hinge-Fc glycoproteins derived from CLM24 cells transformed with: plasmid pMW07-pglΔBCDEF (top panel) or pMW07-pglABICDEF (bottom panel), plasmid pMLBAD encoding DmPglB, and plasmid pTrc99S-hinge-Fc encoding hinge-Fc derived from human IgG1. Glycosylation efficiency estimated to be 29% and 12%, respectively. Reduction with DTT converted glycosylated hinge-Fc dimers to monomers. The signal at m/z=27,803 is the monomeric starting material corresponding to GalNAc5GlcNAc-hinge-Fc that was subjected to chemoenzymatic remodeling. FIG. 17B shows LC-ESI-MS monitoring of the exo-α-N-acetylgalactosaminidase-treated hinge-Fc glycoproteins (right panel) and the EndoS2-D184M-catalyzed reaction with the complex-type Gal₂GlcNAc₂Man₃GlcNAc (G2)-oxazoline (left panel). For G2-hinge-Fc, calculated m/z=28,206 Da and found m/z=28,206 Da. The signal at m/z=26,786 is the precursor material GlcNAc-hinge-Fc.

### DETAILED DESCRIPTION

### General Definitions

Unless otherwise indicated, the definitions and embodiments described in this and other sections are intended to be applicable to all embodiments and aspects of the present application herein described for which they are suitable as would be understood by a person skilled in the art.

As used herein, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

Terms of degree such as “about” and “approximately” as used herein mean a reasonable amount of deviation of the modified term such that the end result is not significantly changed. These terms of degree should be construed as including a deviation of at least ±1% (and up to ±5% or ±10%) of the modified term if this deviation would not negate the meaning of the word it modifies. The allowable variation encompassed by the term “about” or “approximately” may depend on the context.

The term “and/or” as used herein means that the listed items are present, or used, individually or in combination. In effect, this term means that “at least one of” or “one or more” of the listed items is used or present.

As will be understood by a person of ordinary skill in the art, for any and all purposes, such as in terms of providing a written description, all ranges disclosed herein also encompass any and all possible subranges and combinations of subranges thereof, as well as any value within a range. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by a person of ordinary skill in the art all language such as “up to,” “at least,” and the like include the number recited and refer to ranges which can be subsequently broken down into subranges or specific values therein as discussed above. Finally, as will be understood by a person of ordinary skill in the art, and as discussed above, a range includes each individual value.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

As used herein, amino acid residues will be indicated either by their full name or according to the standard three-letter or one-letter amino acid code.

The term “polypeptide,” “peptide”, or “protein” are used interchangeably and to refer to a polymer of amino acid residues. The terms encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “express” and “expression” mean allowing or causing the information in a DNA sequence to become produced, for example producing an RNA by activating the cellular functions involved in transcription of a DNA sequence.

The terms “nucleic acid” and “nucleotide” encompass both DNA and RNA unless specified otherwise.

As used herein, the “DNA constructs” of the disclosure are nucleic acid molecules containing a combination of two or more genetic elements not naturally occurring together. Each DNA construct comprises a non-naturally occurring nucleotide sequence that can be in the form of linear DNA or circular DNA, i.e., placed within a vector.

As used herein, the term “glycan” refers to a complex carbohydrate molecule comprising sugar molecules linked together in a branched or linear form. The term “glycan” is inclusive of both oligosaccharides and polysaccharides and includes both branched and unbranched polymers.

Certain terms employed in the specification, examples, and claims are collected herein. Unless defined otherwise, all technical and scientific terms used herein have the same meaning as commonly understood by one of ordinary skill in the art to which this invention belongs.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Provided herein are embodiments wherein any embodiment described herein may be combined with any one or more other embodiments, provided the combination is not mutually exclusive.

### Recombinant Oligosaccharyltransferase (OST)

Human immunoglobulin G (IgG) antibodies are one of the most important classes of biotherapeutic agents and undergo glycosylation at the conserved N297 site in the CH2 domain, which is critical for IgG Fc effector functions and anti-inflammatory activity. Hence, technologies for producing authentically glycosylated IgGs are in high demand. While attempts to engineer Escherichia coli for this purpose have been described, they have met limited success due in part to the lack of available oligosaccharyltransferase (OST) enzymes that can install N-linked glycans within the QYNST (SEQ ID NO: 5) sequon of the IgG CH2 domain. The Examples (infra) of the present disclosure demonstrate the identification of a previously uncharacterized single-subunit OST (ssOST) from the bacterium Desulfovibrio marinus that exhibited greatly relaxed substrate specificity and, as a result, was able to catalyze glycosylation of native CH2 domains in the context of both a hinge-Fc fragment and a full-length IgG. Although the attached glycans were bacterial in origin, conversion to a homogeneous, asialo complex-type G2 N-glycan at the QYNST (SEQ ID NO: 5) sequon of the E. coli-derived hinge-Fc was achieved via chemoenzymatic glycan remodeling. Importantly, the resulting G2-hinge-Fc exhibited strong binding to human FcγRIIIa (CD16a), one of the most potent receptors for eliciting antibody-dependent cellular cytotoxicity (ADCC). Taken together, the discovery of a unique ssOST from D. marinus provides previously unavailable biocatalytic capabilities to the bacterial glycoprotein engineering toolbox and opens the door to using E. coli for the production and glycoengineering of human IgGs and fragments derived thereof.

Accordingly, one aspect of the present disclosure is directed to a recombinant oligosaccharyltransferase (OST) capable of catalyzing the transfer of a glycan onto a sequon comprising an N−X−T motif, wherein X can be any amino acid.

In accordance with this and all aspects of the present disclosure, the term “oligosaccharyltransferase” refers generally to a glycosylation enzyme or subunit of a glycosylation enzyme complex that is capable of transferring a glycan, i.e., an oligosaccharide or polysaccharide, from a donor substrate to a particular acceptor substrate. The donor substrate is typically a lipid carrier molecule linked to the glycan, and the acceptor substrate is typically a particular amino acid residue of a target glycoprotein. Suitable OSTs include those enzymes that transfer a glycan to an asparagine residue, i.e., an OST involved in N-linked glycosylation, and those enzymes that transfer a glycan or activated sugar moiety to a hydroxyl oxygen molecule of an amino acid residue, i.e., an OST involved in O-linked glycosylation. An OST may be a single-subunit enzyme, a multi-subunit enzyme complex, or a single subunit derived from a multi-subunit enzyme complex. In some embodiments, the OST according to the present disclosure is a single subunit OST.

In accordance with this and all aspect of the present disclosure, the term “sequon” refers to a specific sequence of amino acids in a protein that is recognized by enzymes responsible for glycosylation, particularly N-linked glycosylation (see, e.g., Kornfeld and Kornfeld, “Assembly of Asparagine-Linked Oligosaccharides,” Annu. Rev. Biochem. 54:31-664 (1985), which is hereby incorporated by reference in its entirety). This sequence is crucial for the attachment of carbohydrate groups to proteins, which can affect protein folding, stability, and function.

In some embodiments, the sequon comprises an X₋₂QNX₋₁T (SEQ ID NO: 3) motif, where X₋₂ and X₋₁ can be any amino acid but proline, or an XQNAT (SEQ ID NO: 4) motif, where X can be any amino acid.

In some embodiments, the sequon is selected from the group consisting of QYNST (SEQ ID NO: 5), DQNAT (SEQ ID NO: 6), AENIT (SEQ ID NO: 7), NENIT (SEQ ID NO: 8), LVNSS (SEQ ID NO: 9), SRNLT (SEQ ID NO: 10), QSNDT (SEQ ID NO: 11), FSNTT (SEQ ID NO: 12), PGNAS (SEQ ID NO: 13), QSNST (SEQ ID NO: 14), NFNLT (SEQ ID NO: 15), LGNAT (SEQ ID NO: 16), MENFS (SEQ ID NO: 17), SPNKT (SEQ ID NO: 18), DVNKS (SEQ ID NO: 19), LLNKS (SEQ ID NO: 20), SQNSS (SEQ ID NO: 21), and AQNAT (SEQ ID NO: 22).

TABLE 1
Exemplary Sequon/N-Glycosylation
Acceptor Site Sequences

| | SEQ |
|---|---|
| Sequon/N-Glycosylation | ID |
| Acceptor Site | NO: |
| X₋₂QNX₋₁T, where X₋₂ and X₋₁ can | 3 |
| be any amino acid but proline | |
| XQNAT motif, where X can | 4 |
| be any amino acid | |
| QYNST | 5 |
| DQNAT | 6 |
| AENIT | 7 |
| NENIT | 8 |
| LVNSS | 9 |
| SRNLT | 10 |
| QSNDT | 11 |
| FSNTT | 12 |
| PGNAS | 13 |
| QSNST | 14 |
| NFNLT | 15 |
| LGNAT | 16 |
| MENFS | 17 |
| SPNKT | 18 |
| DVNKS | 19 |
| LINKS | 20 |
| SQNSS | 21 |
| AQNAT | 22 |

The recombinant oligosaccharyltransferase according to the present disclosure is capable of catalyzing glycosylation of an antibody. The term “antibody” refers to a complete immunoglobulin molecule or a functional fragment thereof. Naturally occurring antibodies generally include tetramers, usually composed of at least two heavy (H) chains and at least two light (L) chains. Each heavy (H) chain includes a heavy chain variable (hereinafter referred to as VH) domain and a heavy chain constant (CH) domain. The heavy chain constant domain includes three CH1, CH2, and CH3 constant domains. The heavy chain can be of any isotype, including IgG (IgG1, IgG2, IgG3, and IgG4 subtypes), IgA (IgA1 and IgA2 subtypes), IgM, and IgE. Each light chain includes a light chain variable (hereinafter referred to as VL) domain and a light chain constant (light chain constant, CL) domain. Light chains include kappa (κ) chains and lambda (λ) chains. The combination of VH domain and VL domain is generally responsible for recognizing antigens, while the CH domain can mediate the binding of immunoglobulins to host tissues or factors, including various cells of the immune system (such as effector cells) and the first step of the complement system of the classical pathway. VH and VL domains can be subdivided into highly variable (hypervariability) regions, also known as complementarity determining regions (CDRs), and the CDRs are interspersed with more conserved antibody framework regions (FRs). Each VH and VL domain is composed of three CDRs and four FRs respectively. The order from N-terminus to C-terminus is as follows: FR1, CDR1, FR2, CDR2, FR3, CDR3 and FR4. The heavy and light chain variable regions contain binding domains to interact with antigens.

The recombinant oligosaccharyltransferase according to the present disclosure is capable of catalyzing glycosylation of an antigen-binding fragment of an antibody. The term “antigen-binding fragment” refers to the complete structure or part of an antibody, which may include Fab fragments, Fab′ fragments, F(ab′)2 fragments, Fd fragments, Fv fragments, and disulfide-linked fragments. Fv fragments include single chain variable fragments (scFv), single chain variable fragment dimers ((scFv)₂, also known as diabodies), single chain variable fragment Trimers ((scFv)₃, also known as triabodies), single chain variable region fragment tetramers ((scFv)₄, also known as tetrabodies), single domain antibodies (single domain antibodies, dAb), minibodies, nanobodies, and multispecific antibodies formed from antibody fragments.

The terms “antibody” and “antigen-binding fragment thereof” encompass any modified configuration of the immunoglobulin molecule that comprises an antigen recognition site of the required specificity, including glycosylation variants of antibodies, amino acid sequence variants of antibodies, and covalently modified antibodies.

In some embodiments, the antibody, or antigen-binding fragment thereof, according to the present disclosure is a human antibody, a humanized antibody, or an antigen-binding fragment of a human antibody or humanized antibody. In accordance with such embodiments, the antibody or antigen-binding fragment thereof is an IgG antibody, an IgM antibody, an IgA antibody, an IgE antibody, or an IgD antibody. The antibody or antigen-binding fragment thereof may be an IgG antibody or antigen-binding fragment thereof, e.g., an IgG1 antibody, an IgG2 antibody, an IgG3 antibody, or an IgG4 antibody.

Thus, in some embodiments, the recombinant oligosaccharyltransferase according to the present disclosure is capable of catalyzing glycosylation of human IgG and/or fragments thereof. In accordance with such embodiments, the human IgG and/or fragments thereof may comprise a C_{H}2 domain.

In accordance with this and all aspects of the present disclosure, the glycan may be a prokaryotic glycan. Prokaryotic glycans are diverse carbohydrate structures found in bacteria and archaea, playing crucial roles in cellular processes such as cell wall integrity, signaling, and immune evasion (see, e.g., Moens and Vanderleyden, “Glycoproteins in Prokaryotes,” Arch. Microbiol. 168(3):169-175 (1997), which is hereby incorporated by reference in its entirety). These glycans are often distinct from eukaryotic glycans, offering unique structural features and biosynthetic pathways.

Suitable exemplary prokaryotic glycans include, without limitation, GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAc₂ (Man5), Man₅₋₉GlcNAc₂ (Man5-9 or high mannose glycan), GlcNAc₂Man₃GlcNAc₂ (G0), Ga₁GlcNAc₂Man₃GlcNAc₂ (G1), Gal₂GlcNAc₂Man₃GlcNAc₂ (G2), Sia₁Gal₂GlcNAc₂Man₃GlcNAc₂ (S1G2), Sia₂Gal₂GlcNAc₂Man₃GlcNAc₂ (S2G2), GlcNAc₂Man₃GlcNAc₂(Fuc) (G0F), Gal₁GlcNAc₂Man₃GlcNAc₂(Fuc) (G1F), Gal₂GlcNAc₂Man₃GlcNAc₂(Fuc) (G2F), Sia₁Gal₂GlcNAc₂Man₃GlcNAc₂(FUC) (S1G2F), Sia₂Gal₂GlcNAc₂Man₃GlcNAc₂ (Fuc) (S2G2F), mono-GlcNAc, bacterial capsular polysaccharide (CPS) antigens, and/or bacterial O-antigen polysaccharide (O-PS) antigens.

In accordance with this and all aspects of the present disclosure, the glycan may be a eukaryotic glycan. Eukaryotic glycans are complex carbohydrate structures found on the surfaces of cells and proteins in organisms such as animals, plants, and fungi (see, e.g., Stanley P, Moremen KW, Lewis NE, et al. N-Glycans. In: Varki A, Cummings RD, Esko JD, et al., editors. Essentials of Glycobiology. 4th edition. Cold Spring Harbor (NY): Cold Spring Harbor Laboratory Press; 2022. Chapter 9, which is hereby incorporated by reference in its entirety). These glycans play critical roles in cellular communication, protein folding, and immune response.

In some embodiments, the eukaryotic glycan comprises a GlcNAc₂ core. The GlcNac₂ core may further comprise at least one mannose residue.

Suitable exemplary eukaryotic glycans include, without limitation, GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAc₂ (Man5), Man₅₋₉GlcNAc₂ (Man5-9 or high mannose glycan), GlcNAc₂Man₃GlcNAc₂ (G0), Gal₁GlcNAC₂Man₃GlcNAc₂ (G1), Gal₂GlcNAc₂Man₃GlcNAc₂ (G2), Sia₁Gal₂GlcNAc₂Man₃GlcNAc₂ (S1G2), Sia₂Gal₂GlcNAc₂Man₃GlcNAc₂ (S2G2), GlcNAc₂Man₃GlcNAC₂(Fuc) (G0F), Gal₁GlcNAc₂Man₃GlcNAc₂(Fuc) (G1F), Gal₂GlcNAc₂Man₃GlcNAc₂(Fuc) (G2F), Sia₁Gal₂GlcNAc₂Man₃GlcNAc₂(Fuc) (S1G2F), and/or Sia₂Gal₂GlcNAc₂Man₃GlcNAc₂ (Fuc) (S2G2F).

As described in the Examples of the present disclosure infra, Applicant sought to discover ssOSTs capable of N-glycosylation of the authentic QYNST (SEQ ID NO: 5) sequon in human Fc fragments and full-length IgGs expressed in E. coli. It was hypothesized that uncharacterized PglBs with broader substrate recognition and higher glycosylation efficiency might exist in the genomes of other Desulfobacterota. To test this hypothesis, a collection of 19 PglB homologs was generated by genome mining of Desulfovibrio spp. and screened in E. coli for the ability to glycosylate canonical and non-canonical acceptor sequons in periplasmically expressed acceptor proteins. This screening campaign led to the discovery of a PglB homolog from D. marinus strain DSM 18311 (DmPglB) that could efficiently glycosylate eukaryotic-type N−X−T motifs in different model acceptor proteins regardless of the residue at the −2 position. The Examples (infra) further demonstrate that the relaxed sequon specificity of DmPglB enabled glycosylation of authentic QYNST (SEQ ID NO: 5) sequons in the context of both a human hinge-Fc fragment and a full-length chimeric IgG composed of murine antigen-binding regions (Fv) and human constant domains.

Thus, in some embodiments, the oligosaccharyltransferase is a Desulfovibrio marinus oligosaccharyltransferase.

In accordance with this and all aspects of the present disclosure, the OST may have the amino acid sequence of SEQ ID NO: 1 or an amino acid sequence having at least 85%, 90%, 95%, 96%, 97%, 98%, or 99% sequence identity to SEQ ID NO:1. The amino acid sequence of SEQ ID NO: 1 is shown below.

SEQ ID NO: 1
MIFSREHSIRRDWKALIVTCVIVMLAGMAVRMQELPEWNQAYRVAGEFIMGTHDAYHWLAGAMGF
GSAADAPPSELLRALSHMTGISVGNLGFFLPAIFGGLVGAATVLWAWALGGLEAGLVAGVIATLAP
GYYYRSRLGYYDTDIVTLLFPLLLTFGLAIWLDGSLCDSWVNRFRSAFSKKNGKAVADATKDEGAE
EETAAPDEPDEPRRFFLIWPALLGGFGSWAALWHGYMLTFLQLTVEMLLELVFVAGKRGRRGALLW
GVAAFAAAGFWGLYGTLGAVVAALLAGALPKNIRAKVYSLAPGLLAAAVVLVASGAAESIVVGGSK
FLASYIKPVAQQTAFRGDTGELVFPGIGQSVIEAQNLPLAEVEDRFHPWGWLSLAGIGGFFMLLVL
RPSALFLLPFLAIALSAVKLGTRMAMFGAPAVGLGLGFLFLWIGRAVLGGQSWSRYVLTFILGALA
LGVALPGVSLFLTLPPTPVLSRHHAQALIDLGKEADKSSEVWTWWDWGYATHYYAGLQSFADGGRH
YGEHVFTLGLALTTPSPMQSAQLIQYSAEHNEEPWTEWEKMGLDKTRDELRSLGTEDLHLKPPMPL
YVVATFENIRLSPWICYYGTWDFEKEQGVHARVASIRESENLDWEKGTMTFQDEKEPIEVKSIHVL
SSQGRKDRHYDKNTGPNLILNSESRRYYALDDLAFQSMLTQLLIAPKEFERLDRYFELVYDDFPWV
RVYKVREVPKDAPAKPQTPAVESPEANGTAANATQPTNGTESGENTTQPANTTQ

In some embodiments, the oligosaccharyltransferase is a Desulfovibrio marinus oligosaccharyltransferase and has the amino acid sequence of SEQ ID NO: 1.

As used herein, the term “glycosylation efficiency” refers to the effectiveness or success rate of the glycosylation process. Established methods for determining glycosylation efficiency are well known in the field and are illustrated, for example, in the Examples section of the present disclosure.

In some embodiments, the OST according to the present disclosure has a glycosylation efficiency for a sequon comprising the amino acid sequence of any one of SEQ ID NOs: 3-22 of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or any amount or range therebetween.

Example 3 (infra) of the present disclosure describes that DmPglB glycosylates the AQNAT (SEQ ID NO: 22) sequon with a glycosylation efficiency of 90%, the DQNAT (SEQ ID NO: 6) sequon with a glycosylation efficiency of 90%, and the non-canonical QYNST (SEQ ID NO: 5) sequon with a glycosylation efficiency of 95%. Thus, in some embodiments, the OST according to the present disclosure has a glycosylation efficiency of at least 90%, at least 95%, at least 96%, at least 97%, at least 98%, at least 99%, or 100%, for a given sequon.

The Examples of the present disclosure infra demonstrate that DmPglB promotes glycosylation of the native QYNST (SEQ ID NO: 5) motif in a human hinge-Fc fragment and a full-length, chimeric IgG antibody, with efficiencies that were significantly higher than any of the efficiencies reported previously for PglB-mediated Fc glycosylation in E. coli (Ollis et al., “Substitute Sweeteners: Diverse Bacterial Oligosaccharyltransferases with Unique N-Glycosylation Site Preferences,” Sci. Rep. 5:15237 (2015); Fisher et al., “Production of Secretory and Extracellular N-Linked Glycoproteins in Escherichia Coli,” Appl. Environ. Microbiol. 77(3):871-881 (2011); Schwarz et al., “A Combined Method for Producing Homogeneous Glycoproteins with Eukaryotic N-Glycosylation,” Nat. Chem. Biol. 6(4):264-266 (2010); Schwarz et al., “Relaxed Acceptor Site Specificity of Bacterial Oligosaccharyltransferase In Vivo,” Glycobiology 21(1):45-54 (2011); and Valderrama-Rincon et al., “An Engineered Eukaryotic Protein Glycosylation Pathway in Escherichia Coli,” Nat. Chem. Biol. 8(5):434-436 (2012), which are hereby incorporated by reference in their entirety). Thus, in some embodiments, the OST according to the present disclosure has a glycosylation efficiency greater than PglB OSTs described in Ollis et al., “Substitute Sweeteners: Diverse Bacterial Oligosaccharyltransferases with Unique N-Glycosylation Site Preferences,” Sci. Rep. 5:15237 (2015); Fisher et al., “Production of Secretory and Extracellular N-Linked Glycoproteins in Escherichia Coli,” Appl. Environ. Microbiol. 77(3):871-881 (2011); Schwarz et al., “A Combined Method for Producing Homogeneous Glycoproteins with Eukaryotic N-Glycosylation,” Nat. Chem. Biol. 6(4):264-266 (2010); Schwarz et al., “Relaxed Acceptor Site Specificity of Bacterial Oligosaccharyltransferase In Vivo,” Glycobiology 21(1):45-54 (2011); and Valderrama-Rincon et al., “An Engineered Eukaryotic Protein Glycosylation Pathway in Escherichia Coli,” Nat. Chem. Biol. 8(5):434-436 (2012), which are hereby incorporated by reference in their entirety.

In some embodiments, the OST described herein demonstrates a glycosylation efficiency greater than the glycosylation efficiency of CjPglB. In accordance with such embodiments, the OST has a glycosylation efficiency for a sequon containing the amino acid sequence of any one of SEQ ID NOs: 3-22 that exceeds the efficiency of CjPglB.

### Nucleic Acid Sequences Encoding Recombinant Oligosaccharyltransferases

Another aspect of the present disclosure is directed to a nucleic acid molecule encoding a recombinant oligosaccharyltransferase according to the present disclosure.

A “nucleic acid molecule” refers to a polymeric form of nucleotides of any length, either deoxyribonucleotides or ribonucleotides, or analogs thereof. The nucleic acid molecules according to the present disclosure encode a recombinant oligosaccharyltransferase (OST). Suitable recombinant oligosaccharyltransferase (OST) are disclosed in detail supra.

In some embodiments, the nucleic acid molecule comprises the nucleic acid sequence of SEQ ID NO: 2 or a nucleic acid sequence having at least 85%, 90%, 95%, 96%, 97%, 98%, or 99% sequence identity to SEQ ID NO:2.

SEQ ID NO: 2
ATGATTTTTTCCCGTGAGCACTCTATCCGCCGTGATTGGAAAGCATTAATCGTAACTTGTGTGATT
GTAATGCTGGCAGGTATGGCAGTGCGCATGCAGGAATTGCCCGAGTGGAATCAACCAGCATACCGT
GTAGCAGGTGAATTTATTATGGGCACACATGACGCGTATCACTGGCTTGCAGGGGCGATGGGCTTC
GGGTCAGCTGCTGACGCGCCGCCATCTGAGTTGCTGCGTGCCCTGTCGCACATGACTGGGATCTCC
GTGGGTAACCTTGGGTTCTTTTTGCCTGCGATCTTCGGAGGCTTAGTTGGGGCGGCGACCGTCTTA
TGGGCCTGGGCCCTTGGTGGTTTGGAGGCGGGCCTGGTGGCCGGTGTCATTGCCACGCTGGCGCCT
GGTTACTACTACCGTTCACGTTTGGGGTACTATGACACAGATATCGTCACTCTGTTATTCCCATTG
CTTTTGACATTTGGGCTGGCGATCTGGTTGGATGGTAGCTTATGTGATAGTTGGGTGAACCGCTTT
CGTTCGGCCTTTTCCAAGAAGAACGGAAAAGCTGTCGCTGATGCGACTAAGGATGAAGGCGCGGAG
GAGGAGACAGCCGCTCCAGACGAGCCCGATGAACCACGTCGTTTCTTTTTAATCTGGCCTGCGTTG
TTGGGAGGTTTCGGGTCCTGGGCAGCTCTGTGGCATGGTTACATGTTAACTTTCCTTCAGTTGACG
GTGTTTATGTTGCTTTTTCTGGTATTCGTCGCCGGTAAGCGCGGGCGCCGTGGAGCCTTATTGTGG
GGAGTGGCCGCTTTCGCTGCGGCCGGATTTTGGGGCTTATATGGCACGCTTGGGGCCGTAGTTGCC
GCGCTTCTTGCGGGAGCGCTTCCGAAGAACATCCGTGCCAAAGTGTATTCACTGGCTCCAGGGTTA
TTAGCAGCTGCAGTTGTCTTGGTTGCTTCTGGGGCCGCGGAATCTATCGTTGTAGGTGGATCAAAG
TTTTTGGCTAGTTATATCAAGCCGGTGGCACAACAAACTGCCTTTCGTGGGGATACTGGTGAACTG
GTATTTCCTGGGATTGGGCAATCCGTTATTGAAGCACAGAACCTTCCATTAGCTGAGGTCTTCGAT
CGTTTCCACCCATGGGGATGGCTTTCCCTGGCCGGTATCGGAGGTTTTTTTATGTTACTGGTTCTG
CGCCCGTCCGCTCTGTTTCTGCTTCCTTTCTTAGCCATTGCACTTTCCGCCGTTAAGTTAGGTACC
CGCATGGCCATGTTTGGCGCCCCGGCGGTTGGGTTGGGCCTTGGATTTTTATTCCTTTGGATCGGT
CGTGCCGTGTTGGGTGGACAGAGCTGGTCCCGTTATGTCCTGACGTTCATCCTTGGTGCCCTTGCG
TTGGGGGTCGCGTTACCCGGGGTAAGTTTATTCCTTACACTGCCGCCAACTCCCGTACTGTCGCGC
CACCACGCGCAGGCTTTGATTGACTTGGGCAAGGAGGCTGATAAATCATCGGAAGTGTGGACGTGG
TGGGACTGGGGTTACGCGACGCACTACTACGCTGGACTTCAATCCTTCGCTGATGGGGGACGTCAT
TATGGCGAACACGTCTTTACTTTAGGGCTGGCATTGACAACGCCGAGTCCCATGCAAAGCGCACAA
CTGATTCAGTATTCAGCGGAACACAACGAGGAGCCTTGGACCGAGTGGGAGAAAATGGGCTTGGAC
AAGACCCGTGACTTCTTACGCTCTCTGGGAACTGAAGATCTGCACTTAAAGCCTCCCATGCCACTT
TATGTCGTGGCTACTTTTGAAAACATTCGTCTGTCGCCTTGGATTTGTTATTATGGAACTTGGGAC
TTCGAGAAAGAGCAGGGTGTCCACGCGCGTGTGGCGAGCATTCGCGAGAGTTTTAACTTGGACTGG
GAAAAGGGAACGATGACTTTTCAAGATGAAAAAGAACCCATTGAGGTCAAGTCGATCCATGTTTTG
TCCTCGCAGGGGCGCAAAGACCGTCATTATGATAAAAATACGGGCCCAAACCTTATCTTAAACAGC
GAAAGTCGCCGCTATTACGCGCTGGACGATTTGGCATTCCAATCAATGTTAACTCAGCTTCTTATT
GCCCCTAAGGAATTCGAACGTCTTGACCGCTATTTCGAATTAGTCTATGATGACTTTCCGTGGGTC
CGTGTATACAAGGTTCGCGAGGTACCGAAGGATGCGCCTGCTAAGCCGCAGACACCGGCTGTCGAA
AGTCCGGAAGCTAACGGCACTGCCGCAAATGCTACTCAACCAACTAATGGGACAGAATCCGGCGAG
AACACCACCCAACCAGCTAACACGACACAG

In some embodiments, the nucleic acid molecule comprises the nucleic acid sequence of SEQ ID NO: 2.

### Vectors Encoding Recombinant Oligosaccharyltransferases

Another aspect of the present disclosure is directed to a vector comprising a nucleic acid sequence encoding a recombinant oligosaccharyltransferase according to the present disclosure and a promoter heterologous to the nucleic acid sequence encoding the recombinant oligosaccharyltransferase.

The nucleic acid molecules of the present disclosure may be inserted into “vectors.” The term “vector” is widely used and understood by those of skill in the art to refer to a vehicle that allows or facilitates the transfer of nucleic acid molecules from one environment to another or that allows or facilitates the manipulation of a nucleic acid molecule. Vectors can be linear or circular. Vectors can integrate into a target genome of a host cell or replicate independently in a host cell. Vectors can comprise, e.g., an origin of replication, a multicloning site, and/or a selectable marker.

The term “vector” also includes both viral and nonviral means for introducing a nucleic acid molecule into a cell in vitro, in vivo, or ex vivo. Vectors may be introduced into desired host cells by well-known methods, including, but not limited to, transfection, transduction, cell fusion, and lipofection.

The vector may be an expression vector. The term “expression vector” refers to nucleic acid construct that permits the expression of an mRNA, protein, polypeptide, or peptide by a host cell. In some embodiments, the vector is an expression vector capable of directing the expression of a nucleic acid sequence encoding an oligosaccharyltransferase according to the present disclosure. In accordance with such embodiments, the vector may be a prokaryotic expression vector.

Non-limiting examples of prokaryotic expression vectors include, but are not limited to, plasmids such as pMLBAD vectors, pSF vectors, pET vectors, pBAD vectors, pUC vectors, pBAD vectors, pGEX vectors, and pQE vectors. In some embodiments, the vector is a pMLBAD vector (Lefebre and Valvano, “Construction and Evaluation of Plasmid Vectors Optimized for Constitutive and Regulated Gene Expression in Burkholderia cepacian Complex Isolates,” Appl. Environ. Microbiol. 68(12):5956-5964 (2002), which is hereby incorporated by reference in its entirety) or a pSF vector (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety).

The expression vector may include one or more regulatory sequences, selected on the basis of the cells to be used for expression, which is operably linked to the nucleic acid to be expressed. Within an expression vector, “operably linked” is intended to mean that a nucleic acid sequence of interest is linked to the regulatory sequence(s) in a manner which allows for expression of the nucleic acid sequence (e.g., in an in vitro transcription/translation system or in a cell when the vector is introduced into the cell). Regulatory sequences include promoters, enhancers, and other expression control elements (e.g., polyadenylation signals). Regulatory sequences include those which direct constitutive expression of a nucleic acid in many types of cells, those which direct expression of the nucleic acid sequence only in certain cells (e.g., tissue specific regulatory sequences), and those which direct the expression of the nucleic acid sequence upon stimulation with a particular agent (e.g., inducible regulatory sequences). The design of the expression vector can depend on such factors as the choice of the cell to be transformed, the level of expression of protein desired, etc.

A variety of genetic signals and processing events that control many levels of gene expression (e.g., DNA transcription and messenger RNA (“mRNA”) translation) can be incorporated into the nucleic acid construct to maximize enzyme production. For purposes of expressing a cloned nucleic acid sequence encoding one or more desired enzymes, it is advantageous to use strong promoters to obtain a high level of transcription. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR.

There are other specific initiation signals required for efficient gene transcription and translation in prokaryotic cells that can be included in the nucleic acid construct to maximize peptide production, e.g., the Shine-Dalgarno ribosome binding site. Depending on the vector system and host utilized, any number of suitable transcription and/or translation elements, including constitutive, inducible, and repressible promoters, as well as minimal 5′ promoter elements, enhancers or leader sequences may be used. For a review on maximizing gene expression sec Roberts and Lauer, “Maximizing Gene Expression on a Plasmid Using Recombination In Vitro,” Methods in Enzymology 68:473-82 (1979), which is hereby incorporated by reference in its entirety.

As an alternative to recombinant expression of an oligosaccharyltransferase according to the present disclosure using a cell, an expression vector containing a nucleic acid sequence encoding an oligosaccharyltransferase according to the present disclosure can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce an oligosaccharyltransferase according to the present disclosure.

A nucleic acid molecule encoding an oligosaccharyltransferase or other protein component of the present disclosure (e.g., glycoprotein target, enzymes involved in glycan production), a promoter molecule of choice, including, without limitation, enhancers, and leader sequences, a suitable 3′ regulatory region to allow transcription in the host, and any additional desired components, such as reporter or marker genes, are cloned into the vector of choice using standard cloning procedures in the art, such as described in Joseph Sambrook et al., MOLECULAR CLONING: A LABORATORY MANUAL (Cold Springs Harbor 1989); Frederick M. Ausubel, SHORT PROTOCOLS IN MOLECULAR BIOLOGY (Wiley 1999), and U.S. Pat. No. 4,237,224 to Cohen and Boyer, which are hereby incorporated by reference in their entirety.

### Host Cells Comprising Recombinant Oligosaccharyltransferases, Nucleic Acid Sequences or Vectors According to the Disclosure

Another aspect of the present disclosure is directed to a host cell comprising a recombinant oligosaccharyltransferase, nucleic acid sequence, or vector according to the present disclosure.

Recombinant molecules (e.g., nucleic acid sequences and vectors according to the present disclosure) can be introduced into cells, without limitation, via transfection (if the host is a eukaryote), transduction, conjugation, mobilization, electroporation, lipofection, protoplast fusion, calcium chloride transformation, mobilization, transfection using bacteriophage, or particle bombardment, using standard cloning procedures known in the art, as described by JOSEPH SAMBROOK et al., MOLECULAR CLONING: A LABORATORY MANUAL (Cold Springs Harbor 1989), which is hereby incorporated by reference in its entirety. For bacterial cells, suitable techniques include calcium chloride transformation, electroporation, and transfection using bacteriophage. The cells may be stably or transiently transfected with a nucleic acid sequence comprising a nucleotide sequence encoding an oligosaccharyltransferase according to the present disclosure.

Suitable host cells for recombinant protein production include both prokaryotic host cells and eukaryotic host cells. Suitable prokaryotic host cells include, without limitation, E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix, sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp, Stenotrophomonas sp., Micrococcus sp., Neisseria sp., Bdellovibrio sp., Hemophilus sp., Klebsiella sp., Proteus mirabilis, Enterobacter cloacae, Serratia sp., Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderia sp., Aeromonas sp., Plesiomonas sp., Legionella sp. and alpha-proteobacteria such as Wolbachia sp., cyanobacteria, spirochactes, green sulfur and green non-sulfur bacteria, Gram-negative cocci, Gram negative bacilli which are fastidious, Enterobacteriaceae-glucose-fermenting gram-negative bacilli, Gram negative bacilli-non-glucose fermenters, Gram negative bacilli-glucose fermenting, oxidase positive.

In addition to bacteria cells, eukaryotic cells such as mammalian, insect, and yeast systems are also suitable host cells for transfection/transformation of the expression vector for recombinant protein production. Mammalian cell lines available in the art for expression of a heterologous protein or polypeptide include Chinese hamster ovary cells, HeLa cells, baby hamster kidney cells, COS cells and many others.

In some embodiments, the cells are engineered to constitutively express an oligosaccharyltransferase according to the present disclosure.

In some embodiments, the cells are engineered such that expression of the oligosaccharyltransferase according to the present disclosure may be induced.

The host cell may further comprise a protein of interest. In accordance with this and all aspects of the present disclosure, a “protein of interest” includes any peptide, polypeptide, or protein that comprise one or more glycan acceptor amino acid residues. The one or more glycan acceptor amino acid sites may be an engineered or natural glycan acceptor site. The protein of interest may have 1, 2, 3, 4, 5, 6, 7, 8, 9, or more glycan acceptor amino acid sites. In some embodiments, the protein of interest has a single glycan acceptor amino acid site. In other embodiments, the protein of interest has 2 glycan acceptor amino acid sites or 3 glycan acceptor amino acid sites. In further embodiments, the protein of interest has at least 2 glycan acceptor amino acid sites, at least 3 glycan acceptor amino acid sites, at least 4 glycan acceptor amino acid sites, or at least 5 glycan acceptor amino acid sites.

Suitable exemplary proteins of interest include, without limitation, immunological proteins (immunoglobulins, histocompatibility antigens), hormones, enzymes, cell attachment recognition sites, receptors, protein folding chaperones, developmentally regulated proteins, and proteins involved in hemostasis and thrombosis. In some embodiments, the protein of interest is a therapeutic protein such as an antibody or an antigen-binding fragment thereof.

The protein of interest may be an antibody or an antigen-binding fragment thereof. An antibody, or antigen-binding fragment thereof, according to the present disclosure may be a human antibody, a humanized antibody, or an antigen-binding fragment of a human antibody or humanized antibody. In accordance with such embodiments, the antibody or antigen-binding fragment thereof is an IgG antibody, an IgM antibody, an IgA antibody, an IgE antibody, or an IgD antibody. In some embodiments, the antibody or antigen-binding fragment thereof is an IgG antibody or antigen-binding fragment thereof. Thus, the antibody may be an IgG1 antibody, an IgG2 antibody, an IgG3 antibody, or an IgG4 antibody. In some embodiments, the antibody antigen-binding fragment thereof is an antigen-binding fragment of an IgGI antibody, an IgG2 antibody, an IgG3 antibody, or an IgG4 antibody.

The antibody or antigen-binding fragment of the present disclosure may be a human IgG antibody or ah antigen-binding fragment of a human IgG antibody. In accordance with such embodiments, the human IgG or antigen-binding fragment thereof may be of IgG1, IgG2, IgG3, or IgG4 isotype.

The antibody, or antigen-binding fragment thereof, according to the present disclosure may be a mouse antibody or an antigen-binding fragment of a mouse antibody. In accordance with such embodiments, the antibody or antigen-binding fragment thereof is an IgG antibody, IgM antibody, IgA antibody, IgE antibody, or IgD antibody. In some embodiments, the antibody or antigen-binding fragment thereof is a mouse IgG antibody or antigen-binding fragment thereof. Thus, in some embodiments, the antibody is an IgG1 antibody, an IgG2a antibody, an IgG2b antibody, an IgG2c antibody, or an IgG3 antibody. In some embodiments, the antibody antigen-binding fragment thereof is an antigen-binding fragment of an IgGI antibody, an IgG2a antibody, an IgG2b antibody, an IgG2c antibody, or an IgG3 antibody.

The antibody, or antigen-binding fragment thereof, according to the present disclosure may be chimeric antibody or an antigen-binding fragment of a chimeric antibody. The chimeric antibody or chimeric antigen-binding fragment thereof may include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, the chimeric antibody or antigen-binding fragment thereof comprises a mouse variable region or part of a mouse variable region and a human constant region or portion of a human constant region. In some embodiments, amino acid modifications can be made in the variable region and/or the constant region.

An antibody according to the present disclosure may be a full-length antibody. In accordance with such embodiments, the full-length antibody comprises two heavy chains and two light chains, each including a variable domain and a constant domain.

The antigen-binding fragment according to the present disclosure can comprise or be an antigen-binding fragment of a full-length antibody. Examples of antigen-binding fragments encompassed within the term “antigen-binding fragment” include (i) a Fab fragment comprising a variable light chain (VL) domain, a variable heavy chain (VH) domain, constant light chain (CL) domain, and a fist constant heavy chain (CH1) domain; (ii) a F(ab′)2 fragment, a bivalent fragment comprising two Fab fragments linked by a disulfide bridge at the hinge region; (iii) a Fd fragment comprising the VH and CHI domains of a heavy chain; (iv) a Fv fragment consisting of the VL and VH domains of a single arm of an antibody, (v) a dAb fragment (Ward et al., “Binding Activities of a Repertoire of Single Immunoglobulin Variable Domains Secreted from Escherichia coli,” Nature 341(6242):544-546 (1989), which is hereby incorporated by reference in its entirety), comprising a VH domain; (vi) an isolated CDR that retains functionality; and (vii) single-chain variable fragment (scFv) comprising the VH and VL domains of an antibody joined together by a flexible polypeptide linker (see, e.g., Bird et al., “Single-Chain Antigen-Binding Proteins,” Science 242(4877):423-426(1988); and Huston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85(16):5879-5883 (1988), which are hereby incorporated by reference in their entirety). In some embodiments, the antibody, or antigen-binding fragment thereof, is a fragment antigen binding (Fab) fragment, a Fd fragment, a F(ab′)2 fragment, a variable fragment (Fv), a single chain variable fragment (scFv), or similar constructs utilizing CDRs, VH, and/or VL sequences.

In some embodiments, the protein of interest is a fragment of human IgG, where the fragment is C_{H}2, C_{H}2-C_{H}3, hinge-C_{H}2, hinge-C_{H}2-C_{H}3, fragment crystallizable (Fc) domain, a single-chain variable fragment (scFv), single-chain antibody (scAb), single-domain antibody (scAb), Fab, and/or V_{H}/V_{L} variable regions.

Antibody heavy chain constant region sequences are well known in the art (see, e.g., Wurzburg et al., “Structure of the Human IgE-Fc Cε3-Cε4 Reveals Conformational Flexibility in the Antibody Effector Domains,” Immunity 13(3):375-385 (2000), which is hereby incorporated by reference in its entirety).

In some embodiments, the antibody or antigen-binding fragment thereof comprises a C_{H}2 domain, a C_{H}2-C_{H}3 fragment, a hinge-C_{H}2 fragment, or a hinge-C_{H}2-C_{H}3 fragment of a human IgG1, IgG2, IgG3, or IgG4 antibody. The amino acid sequence of CH₂ domains, C_{H}2-C_{H}3 fragments, hinge-C_{H}2 fragments, and hinge-C_{H}2-C_{H}3 fragments corresponding to human IgG1, IgG2, IgG3, and IgG4 are shown in Tables 2-5 below.

TABLE 2
Human IgG1 Antibody Sequences

| | | SEQ |
|---|---|---|
| Description | Sequence (UniProt Accession No. P01857) | ID NO: |
| C_{H}2 | PCPAPELLGGPSVFLFPPKPKDTLMISRTPEVTCVVVDVSHEDP | 41 |
| | EVKFNWYVDGVEVHNAKTKPREEQYNSTYRVVSVLTVLHQDWLN | |
| | GKEYKCKVSNKALPAPIEKTISKAK | |
| C_{H}2-C_{H}3 | PCPAPELLGGPSVFLFPPKPKDTLMISRTPEVTCVVVDVSHEDP | 42 |
| | EVKFNWYVDGVEVHNAKTKPREEQYNSTYRVVSVLTVLHQDWLN | |
| | GKEYKCKVSNKALPAPIEKTISKAKgqprepqvytlppsrdelt | |
| | knqvsltclvkgfypsdiavewesngqpennykttppvldsdgs | |
| | fflyskltvdksrwqqgnvfscsvmhealhnhytqkslslspel | |
| | (C_{H}2 shown in uppercase; C_{H}3 shown in lowercase) | |
| hinge-C_{H}2 | EPKSCDKTHTCPPCPAPELLGGPSVFLFPPKPKDTLMISRTPEV | 43 |
| | TCVVVDVSHEDPEVKENWYVDGVEVHNAKTKPREEQYNSTYRVV | |
| | SVLTVLHQDWLNGKEYKCKVSNKALPAPIEKTISKAK | |
| | (Hinge shown in bold; C_{H}2 shown in uppercase) | |
| hinge-C_{H}2- | EPKSCDKTHTCPPCPAPELLGGPSVFLFPPKPKDTLMISRTPEV | 44 |
| C_{H}3 | TCVVVDVSHEDPEVKENWYVDGVEVHNAKTKPREEQYNSTYRVV | |
| | SVLTVLHQDWLNGKEYKCKVSNKALPAPIEKTISKAKgqprepq | |
| | vytlppsrdeltknqvsltclvkgfypsdiavewesngqpenny | |
| | kttppvldsdgsfflyskltvdksrwqqgnvfscsvmhealhnh | |
| | ytqkslslspel | |
| | (Hinge shown in bold; C_{H}2 shown in uppercase; | |
| | C_{H}3 shown in lowercase) | |

TABLE 3
Human IgG2 Antibody Sequences

| | | SEQ |
|---|---|---|
| Description | Sequence (UniProt Accession No. P01859) | ID NO: |
| C_{H}2 | APPVAGPSVFLFPPKPKDTLMISRTPEVTCVVVDVSHEDPEVQF | 45 |
| | NWYVDGVEVHNAKTKPREEQFNSTFRVVSVLTVVHQDWLNGKEY | |
| | KCKVSNKGLPAPIEKTISKTK | |
| C_{H}2-C_{H}3 | APPVAGPSVFLFPPKPKDTLMISRTPEVTCVVVDVSHEDPEVQF | 46 |
| | NWYVDGVEVHNAKTKPREEQFNSTFRVVSVLTVVHQDWLNGKEY | |
| | KCKVSNKGLPAPIEKTISKTKgqprepqvytlppsreemtknqv | |
| | sltclvkgfypsdisvewesngqpennykttppmldsdgsffly | |
| | skltvdksrwqqgnvfscsvmhealhnhytqkslslspel | |
| | (C_{H}2 shown in uppercase; C_{H}3 shown in lowercase) | |
| hinge-C_{H}2 | ERKCCVECPPCPAPPVAGPSVFLFPPKPKDTLMISRTPEVTCVV | 47 |
| | VDVSHEDPEVQFNWYVDGVEVHNAKTKPREEQFNSTFRVVSVLT | |
| | VVHQDWLNGKEYKCKVSNKGLPAPIEKTISKTK | |
| | (Hinge shown in bold; C_{H}2 shown in uppercase) | |
| hinge-C_{H}2- | ERKCCVECPPCPAPPVAGPSVFLFPPKPKDTLMISRTPEVTCVV | 48 |
| C_{H}3 | VDVSHEDPEVQFNWYVDGVEVHNAKTKPREEQFNSTFRVVSVLT | |
| | VVHQDWLNGKEYKCKVSNKGLPAPIEKTISKTKgqprepqvytl | |
| | ppsreemtknqvsltclvkgfypsdisvewesngqpennykttp | |
| | pmldsdgsfflyskltvdksrwqqgnvfscsvmhealhnhytqk | |
| | slslspel | |
| | (Hinge shown in bold; C_{H}2 shown in uppercase; | |
| | C_{H}3 shown in lowercase) | |

TABLE 4
Human IgG3 Antibody Sequences

| | | SEQ |
|---|---|---|
| Description | Sequence (UniProt Accession No. P01860) | ID NO: |
| C_{H}2 | APELLGGPSVFLFPPKPKDTLMISRTPEVTCVVVDVSHEDPEVQ | 49 |
| | FKWYVDGVEVHNAKTKPREEQYNSTFRVVSVLTVLHQDWLNGKE | |
| | YKCKVSNKALPAPIEKTISKTK | |
| C_{H}2-C_{H}3 | APELLGGPSVFLFPPKPKDTLMISRTPEVTCVVVDVSHEDPEVQ | 50 |
| | FKWYVDGVEVHNAKTKPREEQYNSTFRVVSVLTVLHQDWLNGKE | |
| | YKCKVSNKALPAPIEKTISKTKgqprepqvytlppsreemtknq | |
| | vsltclvkgfypsdiavewessgqpennynttppmldsdgsffl | |
| | yskltvdksrwqqgnifscsvmhealhnrftqkslslspe | |
| | (C_{H}2 shown in uppercase; C_{H}3 shown in lowercase) | |
| hinge-C_{H}2 | ELKTPLGDTTHTCPRCPEPKSCDTPPPCPRCPEPKSCDTPPPCP | 51 |
| | RCPEPKSCDTPPPCPRCPAPELLGGPSVFLFPPKPKDTLMISRT | |
| | PEVTCVVVDVSHEDPEVQFKWYVDGVEVHNAKTKPREEQYNSTF | |
| | RVVSVLTVLHQDWLNGKEYKCKVSNKALPAPIEKTISKTK | |
| | (Hinge shown in bold; C_{H}2 shown in uppercase) | |
| hinge-C_{H}2- | ELKTPLGDTTHTCPRCPEPKSCDTPPPCPRCPEPKSCDTPPPCP | 52 |
| C_{H}3 | RCPEPKSCDTPPPCPRCPAPELLGGPSVFLFPPKPKDTLMISRT | |
| | PEVTCVVVDVSHEDPEVQFKWYVDGVEVHNAKTKPREEQYNSTF | |
| | RVVSVLTVLHQDWLNGKEYKCKVSNKALPAPIEKTISKTKgqpr | |
| | epqvytlppsreemtknqvsltclvkgfypsdiavewessgqpe | |
| | nnynttppmldsdgsfflyskltvdksrwqqgnifscsvmheal | |
| | hnrftqkslslspe | |
| | (Hinge shown in bold; C_{H}2 shown in uppercase; | |
| | C_{H}3 shown in lowercase) | |

TABLE 5
Human IgG4 Antibody Sequences

| | | SEQ |
|---|---|---|
| Description | Sequence (UniProt Accession No. P01861) | ID NO: |
| C_{H}2 | APEFLGGPSVFLFPPKPKDTLMISRTPEVTCVVVDVSQEDPEVQ | 53 |
| | FNWYVDGVEVHNAKTKPREEQFNSTYRVVSVLTVLHQDWLNGKE | |
| | YKCKVSNKGLPSSIEKTISKAK | |
| C_{H}2-C_{H}3 | APEFLGGPSVFLFPPKPKDTLMISRTPEVTCVVVDVSQEDPEVQ | 54 |
| | FNWYVDGVEVHNAKTKPREEQFNSTYRVVSVLTVLHQDWLNGKE | |
| | YKCKVSNKGLPSSIEKTISKAKgqprepqvytlppsqeemtknq | |
| | vsltclvkgfypsdiavewesngqpennykttppvldsdgsffl | |
| | ysrltvdksrwqegnvfscsvmhealhnhytqkslslslel | |
| | (C_{H}2 shown in uppercase; C_{H}3 shown in lowercase) | |
| hinge-C_{H}2 | ESKYGPPCPSCPAPEFLGGPSVFLFPPKPKDTLMISRTPEVTCV | 55 |
| | VVDVSQEDPEVQFNWYVDGVEVHNAKTKPREEQFNSTYRVVSVL | |
| | TVLHQDWLNGKEYKCKVSNKGLPSSIEKTISKAK | |
| | (Hinge shown in bold; C_{H}2 shown in uppercase) | |
| hinge-C_{H}2- | ESKYGPPCPSCPAPEFLGGPSVFLFPPKPKDTLMISRTPEVTCV | 56 |
| C_{H}3 | VVDVSQEDPEVQFNWYVDGVEVHNAKTKPREEQFNSTYRVVSVL | |
| | TVLHQDWLNGKEYKCKVSNKGLPSSIEKTISKAKgqprepqvyt | |
| | lppsqeemtknqvsltclvkgfypsdiavewesngqpennyktt | |
| | ppvldsdgsfflysrltvdksrwqegnvfscsvmhealhnhytq | |
| | kslslslel | |
| | (Hinge shown in bold; C_{H}2 shown in uppercase; | |
| | CH3 shown in lowercase) | |

Suitable exemplary proteins of interest include, without limitation, an antibody or a derivative thereof including a fragment crystallizable (Fc) domain, a single-chain variable fragment (scFv), a single-chain antibody (scAb), a single-domain antibody (scAb), a Fab, V_{H}/V_{L} variable regions, a Fc domain (QYNST (SEQ ID NO: 5)), a human EPO (AENIT (SEQ ID NO: 7), NENIT (SEQ ID NO: 8), LVNSS (SEQ ID NO: 9)), a Rnase A (SRNLT (SEQ ID NO: 10)), Fab domains (e.g., Cetuximab, QSNDT (SEQ ID NO: 11), or Etanercept, FSNTT (SEQ ID NO: 12)/PGNAS (SEQ ID NO: 13)), Alpha-1-antitrypsin QSNST (SEQ ID NO: 14), NFNLT (SEQ ID NO: 15), LGNAT (SEQ ID NO: 16), CRM197 vaccine carrier MENFS (SEQ ID NO: 17), SPNKT (SEQ ID NO: 18), DVNKS (SEQ ID NO: 19), PD vaccine carrier LLNKS (SEQ ID NO: 20), and Murine Tnfa SQNSS (SEQ ID NO: 21).

### Recombinant Oligosaccharyltransferases Produced by Host Cells

Another aspect of the present disclosure is directed to a glycoprotein produced by the host cell according to the present disclosure.

Once an oligosaccharyltransferase according to the present disclosure has been produced, it may be isolated or purified by any method known in the art for isolation or purification of a protein, for example, by chromatography (e.g., ion exchange, affinity, reverse phase, hydrophobic interaction, particularly by affinity for the specific antigen, by Protein A, and sizing column chromatography), centrifugation, differential solubility, or by any other standard technique for the isolation or purification of proteins.

In some embodiments, the OST is produced in purified form (e.g., at least about 70 to about 75% pure, at least about 80% to 85% pure, or least about 90% or 95% pure) by conventional techniques. Depending on whether the recombinant host cell is made to secrete the protein into growth medium (see U.S. Pat. No. 6,596,509 to Bauer et al., which is hereby incorporated by reference in its entirety), the protein can be isolated and purified by centrifugation (to separate cellular components from supernatant containing the secreted protein) followed by sequential ammonium sulfate precipitation of the supernatant. The fraction containing the protein can be subjected to gel filtration in an appropriately sized dextran or polyacrylamide column to separate the protein from other cellular components and proteins. If necessary, the protein fraction may be further purified by HPLC.

### Methods of Producing Recombinant Oligosaccharyltransferases

A further aspect of the present disclosure is directed to a method of producing a glycosylated protein. This method involves providing a prokaryotic host cell expressing a heterologous prokaryotic oligosaccharyltransferase enzyme capable of transferring a glycan to an N-glycosylation acceptor site of a protein, said acceptor site comprising an N−X−T motif, where X can be any amino acid but proline, and culturing the prokaryotic host cell under conditions effective to produce a glycosylated protein.

The N-glycosylation acceptor site of the protein may comprise an X₋₂QNX₋₁T (SEQ ID NO: 3) motif, where X₋₂ and X₋₁ can be any amino acid but proline, or an XQNAT (SEQ ID NO: 4) motif, wherein X can be any amino acid. In some embodiments, the N-glycosylation acceptor sites of the protein is selected from the group consisting of QYNST (SEQ ID NO: 5), DQNAT (SEQ ID NO: 6), AENIT (SEQ ID NO: 7), NENIT (SEQ ID NO: 8), LVNSS (SEQ ID NO: 9), SRNLT (SEQ ID NO: 10), QSNDT (SEQ ID NO: 11), FSNTT (SEQ ID NO: 12), PGNAS (SEQ ID NO: 13), QSNST (SEQ ID NO: 14), NFNLT (SEQ ID NO: 15), LGNAT (SEQ ID NO: 16), MENFS (SEQ ID NO: 17), SPNKT (SEQ ID NO: 18), DVNKS (SEQ ID NO: 19), LLNKS (SEQ ID NO: 20), SQNSS (SEQ ID NO: 21), and AQNAT (SEQ ID NO: 22).

The recombinant oligosaccharyltransferase according to the present disclosure is capable of catalyzing glycosylation of an antibody and/or an antigen-binding fragment of an antibody. Exemplary antibodies and antigen-binding fragments thereof are provided supra. In some embodiments, the oligosaccharyltransferase is capable of catalyzing glycosylation of human IgG and/or an antigen-binding fragment thereof.

The human IgG and/or antigen-binding fragments thereof may comprise a C_{H}2 domain.

In some embodiments of the methods and systems according to the present disclosure, the glycan is a prokaryotic glycan. Exemplary prokaryotic glycans are provided supra and may be selected from the group consisting of GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAC₂ (Man3 or trimmanosyl core glycan), Man₅GlcNAC₂ (Man5), Man₅₋₉GlcNAc₂ (Man5-9 or high mannose glycan), GlcNAc₂Man₃GlcNAc₂ (G0), Gal₁GlcNAc₂Man₃GlcNAc₂ (G1), Gal₂GlcNAc₂Man₃GlcNAc₂ (G2), Sia₁Gal₂GlcNAc₂Man₃GlcNAc₂ (S1G2), Sia₂Gal₂GlcNAc₂Man₃GlcNAc₂ (S2G2), GlcNAc₂Man₃GlcNAc₂(Fuc) (G0F), Gal₁GlcNAc₂Man₃GlcNAC₂(Fuc) (G1F), Gal₂GlcNAc₂Man₃GlcNAc₂(Fuc) (G2F), Sia₁Gal₂GlcNAc₂Man₃GlcNAc₂(Fuc) (S1G2F), Sia₂Gaₗ2GlcNAc₂Man₃GlcNAc₂ (Fuc) (S2G2F), mono-GlcNAc, bacterial capsular polysaccharide (CPS) antigens, and/or bacterial O-antigen polysaccharide (O-PS) antigens.

In some embodiments of the methods and systems according to the present disclosure, the glycan is a eukaryotic glycan. Exemplary eukaryotic glycans are provided supra and may be selected from the group consisting of GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAC₂ (Man3 or trimmanosyl core glycan), Man₅GlcNAc₂ (Man5), Man₅₋₉GlcNAc₂ (Man5-9 or high mannose glycan), GlcNAc₂Man₃GlcNAc₂ (G0), Gal₁GlcNAc₂Man₃GlcNAc₂ (G1), Gal₂GlcNAc₂Man₃GlcNAc₂ (G2), Sia₁Gal₂GlcNAc₂Man₃GlcNAc₂ (S1G2), Sia₂Gal₂GlcNAc₂Man₃GlcNAc₂ (S2G2), GlcNAc₂Man₃GlcNAc₂(Fuc) (G0F), Gal₁GlcNAc₂Man₃GlcNAc₂(Fuc) (G1F), Gal₂GlcNAc₂Man₃GlcNAc₂(Fuc) (G2F), Sia₁Gal₂GlcNAc₂Man₃GlcNAc₂(Fuc) (S1G2F), and/or Sia₂Gal₂GlcNAc₂Man₃GlcNAc₂(Fuc) (S2G2F).

In some embodiments, the oligosaccharyltransferase is a single subunit OST.

As described herein, the oligosaccharyltransferase may be a Desulfovibrio marinus oligosaccharyltransferase.

In some embodiments of the methods and systems according to the present disclosure, the oligosaccharyltransferase is DmPglB.

The oligosaccharyltransferase may comprise the amino acid sequence of SEQ ID NO: 1, or an amino acid sequence having at least 85%, 90%, 95%, 96%, 97%, 98%, or 99% sequence identity to SEQ ID NO:1.

Suitable prokaryotic host cells for use in the methods and systems according to the present disclosure are provided supra. In some embodiments, the prokaryotic host cell is selected from the group consisting of E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix, sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp, Stenotrophomonas sp., Micrococcus sp., Neisseria sp., Bdellovibrio sp., Hemophilus sp., Klebsiella sp., Proteus mirabilis, Enterobacter cloacae, Serratia sp., Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderiasp., Aeromonas sp., Plesiomonas sp., Legionella sp. and alpha-proteobacteria such as Wolbachia sp., cyanobacteria, spirochactes, green sulfur and green non-sulfur bacteria, Gram-negative cocci, Gram negative bacilli which are fastidious, Enterobacteriaceae-glucose-fermenting gram-negative bacilli, Gram negative bacilli-non-glucose fermenters, Gram negative bacilli-glucose fermenting, oxidase positive.

In some embodiments, the prokaryotic host cell is an E. coli host cell. Suitable E. coli host cells are well known in the art. In accordance with this and all aspects of the present disclosure, the E. coli host cell is E. coli strain CLM24, JUDE-1, BL21 (DE3), a variant of BL21, SHuffle and all of its variations, CyDisCo and derivatives, FÅ113 and derivatives, Origami and derivatives, BW25113 and derivatives, MG1655 and derivatives, W3110 and derivatives, AF1000 and derivatives, Rosetta and derivatives, Rosetta-gami B strains, KS272 and derivatives, Lemo21(DE3), NiCo21(DE3), Tuner(DE3), BLR (DE3), or KRX.

In accordance with this and all aspects of the present disclosure, the host cell does not comprise a native oligosaccharyltransferase activity.

In some embodiments, the heterologous oligosaccharyltransferase enzyme is encoded by a nucleic acid molecule comprising the nucleotide sequence of SEQ ID NO: 2, or a nucleic acid sequence having at least 85%, 90%, 95%, 96%, 97%, 98%, or 99% sequence identity to SEQ ID NO:2.

The prokaryotic host cell may further express a heterologous protein of interest. Suitable exemplary proteins of interest are provided supra. In some embodiments, the protein of interest is selected from the group consisting of an antibody, an antibody, a monoclonal IgG1 antibody or derivative thereof including fragment crystallizable (Fc) domain, a single-chain variable fragment (scFv), a single-chain antibody (scAb), a single-domain antibody (scAb), a Fab, V_{H}/V_{L} variable regions, a Fc domain (QYNST (SEQ ID NO: 5)), a human EPO (AENIT (SEQ ID NO: 7), NENIT (SEQ ID NO: 8), LVNSS (SEQ ID NO: 9)), a Rnase A (SRNLT (SEQ ID NO: 10)), Fab domains (e.g., Cetuximab, QSNDT (SEQ ID NO: 11), or Etanercept, FSNTT (SEQ ID NO: 12)/PGNAS (SEQ ID NO: 13)), Alpha-1-antitrypsin QSNST (SEQ ID NO: 14), NFNLT (SEQ ID NO: 15), LGNAT (SEQ ID NO: 16), CRM197 vaccine carrier MENFS (SEQ ID NO: 17), SPNKT (SEQ ID NO: 18), DVNKS (SEQ ID NO: 19), PD vaccine carrier LLNKS (SEQ ID NO: 20), and Murine Tnfa SQNSS (SEQ ID NO: 21).

In some embodiments, the protein of interest is an antibody or an antigen-binding fragment thereof. For example, the antibody or antigen-binding fragment thereof is a human IgG or fragment thereof. In accordance with such embodiments, the antibody or antigen-binding fragment thereof is a human IgG or an antigen-binding fragment thereof.

As described in more detail supra, the human IgG or fragment thereof is of IgG1, IgG2, IgG3, or IgG4 isotype.

In some embodiments, the protein of interest is a fragment of human IgG, wherein the fragment is C_{H}2, C_{H}2-C_{H}3, hinge-C_{H}2, hinge-C_{H}2-C_{H}3, fragment crystallizable (Fc) domain, single-chain variable fragment (scFv), single-chain antibody (scAb), single-domain antibody (scAb), Fab, and/or V_{H}/V_{L} variable regions.

In accordance with this an all aspects of the present disclosure, the protein of interest is selected from the group consisting of scFv13, YebF, RNase A, hinge-Fc, and full-length IgG.

In accordance with this an all aspects of the present disclosure, the prokaryotic host cell lacks a native glycosylation pathway. In accordance with such embodiments, the prokaryotic host cell may be E. coli strain CLM24.

In some embodiments, the prokaryotic host cell may further express a heterologous glycosylation pathway. For example, the prokaryotic host cell may further expresses GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAC₂ (Man5), Man₅₋₉GlcNAc₂ (Man5-9 or high mannose glycan), GlcNAc₂Man₃GlcNAc₂ (G0), Gal₁GlcNAc₂Man₃GlcNAc₂ (G1), Gal₂GlcNAc₂Man₃GlcNAc₂ (G2), Sia₁Gal₂GlcNAc₂Man₃GlcNAc₂ (S1G2), Sia₂Gal₂GlcNAc₂Man₃GlcNAc₂ (S2G2), GlcNAc₂Man₃GlcNAc₂(Fuc) (G0F), Gal₁GlcNAc₂Man₃GlcNAc₂(Fuc) (G1F), Gal₂GlcNAc₂Man₃GlcNAc₂(Fuc) (G2F), Sia₁Gal₂GlcNAc₂Man₃GlcNAc₂(Fuc) (S1G2F), Sia₂Gal₂GlcNAc₂Man₃GlcNAc₂ (Fuc) (S2G2F), mono-GlcNAc, bacterial capsular polysaccharide (CPS) antigens, and/or bacterial O-antigen polysaccharide (O-PS) antigens.

In some embodiments, the glycosylated protein comprises an N-linked GalNac₅GlcNAc. In accordance with such embodiments, the method may further comprise removing GalNAc from the N-linked GalNac₅GlcNAc.

In some embodiments, said removing comprises subjecting the glycosylated protein to enzymatic trimming with an exo-α-N-acetylglycosamineidase to form a GlcNAc stump. In accordance with such embodiments, the method may further comprise transglycosylating the GlcNAc stump. In some embodiments, said transglycosylating is catalyzed by EndoS2-D184M with a G2-oxaxoline as a donor substrate to produce a glycosylated protein comprising Gal₂GlcNAc₂Man₃GlcNAc₂, EndoF3 D165A, Endo-S D233Q, Endo-CC1 N180H, and/or Endo-M N175Q.

### Systems Comprising Recombinant Oligosaccharyltransferases

Yet another aspect of the present disclosure is directed to a system comprising: a first plasmid encoding enzymes for N-glycan biosynthesis; a second plasmid encoding a recombinant oligosaccharyltransferase (OST) according to the present disclosure; and/or a third plasmid encoding a protein of interest.

Exemplary recombinant OSTs according to the present disclosure are provided supra. In some embodiments, the oligosaccharyltransferase is DmPglB. In accordance with such embodiments, the oligosaccharyltransferase may comprise the amino acid sequence of SEQ ID NO: 1, or an amino acid sequence having at least 85%, 90%, 95%, 96%, 97%, 98%, or 99% sequence identity to SEQ ID NO:1.

Exemplary proteins of interest are provided supra. In some embodiments of the system according to the present disclosure, the protein of interest is a glycoprotein target.

The protein of interest may be selected from the group consisting of an antibody, a monoclonal IgGI antibody or derivative thereof including fragment crystallizable (Fc) domain, a single-chain variable fragment (scFv), a single-chain antibody (scAb), a single-domain antibody (scAb), a Fab, V_{H}/V_{L} variable regions, a Fc domain (QYNST (SEQ ID NO: 5)), a human EPO (AENIT (SEQ ID NO: 7), NENIT (SEQ ID NO: 8), LVNSS (SEQ ID NO: 9)), a Rnase A (SRNLT (SEQ ID NO: 10)), Fab domains (eg. Cetuximab, QSNDT (SEQ ID NO: 11), or Etanercept, FSNTT (SEQ ID NO: 12)/PGNAS (SEQ ID NO: 13)), Alpha-1-antitrypsin QSNST (SEQ ID NO: 14), NFNLT (SEQ ID NO: 15), LGNAT (SEQ ID NO: 16), CRM197 vaccine carrier MENFS (SEQ ID NO: 17), SPNKT (SEQ ID NO: 18), DVNKS (SEQ ID NO: 19), PD vaccine carrier LINKS (SEQ ID NO: 20), and Murine Tnfa SQNSS (SEQ ID NO: 21).

In some embodiments of the system according to the present disclosure, the protein of interest is an antibody or a fragment thereof. For example, the antibody or antigen-binding fragment thereof is a human IgG or fragment thereof. The human IgG or antigen-binding fragments thereof may be of IgG1, IgG2, IgG3, or IgG4 isotype.

In some embodiments of the system according to the present disclosure, the protein of interest is a fragment of human IgG, wherein the fragment is C_{H}2, C_{H}2-C_{H}3, hinge-C_{H}2, hinge-C_{H}2-C_{H}3, fragment crystallizable (Fc) domain, a single-chain variable fragment (scFv), single-chain antibody (scAb), single-domain antibody (scAb), Fab, and/or V_{H}/V_{L} variable regions.

The protein of interest may be selected from the group consisting of scFv13, YebF, RNase A, hinge-Fc, and full-length IgG.

The protein of interest may comprise a natural or engineered N-glycan acceptor site.

In some embodiments, the protein of interest comprises an N−X−T motif, wherein X can be any amino acid. In accordance with such embodiments, the protein of interest comprises an X₋₂QNX₋₁T (SEQ ID NO: 3) motif, where X₋₂ and X₋₁ can be any amino acid but proline, or an XQNAT (SEQ ID NO: 4) motif, wherein X can be any amino acid.

In some embodiments, the protein of interest comprises a sequon selected from the group consisting of QYNST (SEQ ID NO: 5), DQNAT (SEQ ID NO: 6), AENIT (SEQ ID NO: 7), NENIT (SEQ ID NO: 8), LVNSS (SEQ ID NO: 9), SRNLT (SEQ ID NO: 10), QSNDT (SEQ ID NO: 11), FSNTT (SEQ ID NO: 12), PGNAS (SEQ ID NO: 13), QSNST (SEQ ID NO: 14), NFNLT (SEQ ID NO: 15), LGNAT (SEQ ID NO: 16), MENFS (SEQ ID NO: 17), SPNKT (SEQ ID NO: 18), DVNKS (SEQ ID NO: 19), LLNKS (SEQ ID NO: 20), SQNSS (SEQ ID NO: 21), and AQNAT (SEQ ID NO: 22).

### EXAMPLES

### Materials and Methods for Examples 1-8

### Bacterial Strains, Growth Conditions, and Plasmids

Escherichia coli strain DH5α was employed for all cloning and library construction. E. coli strain CLM24 (Simmons et al., “Expression of Full-Length Immunoglobulins in Escherichia coli: Rapid and Efficient Production of Aglycosylated Antibodies,” J. Immunol. Methods 263(1-2):133-147 (2002), which is hereby incorporated by reference in its entirety) was utilized for all in vivo glycosylation studies, except for full-length IgG expression and glycosylation, which used E. coli strain JUDE-1 (Mazor et al., “Isolation of Engineered, Full-Length Antibodies from Libraries Expressed in Escherichia coli,” Nat. Biotechnol. 25(5):563-565 (2007), which is hereby incorporated by reference in its entirety). E. coli strain BL21 (DE3) was used to generate acceptor proteins for in vitro glycosylation experiments. Cultures were grown overnight and subsequently subcultured at 37° C. in Luria-Bertani (LB) broth, supplemented with antibiotics as required at the following concentrations: 20 μg/ml chloramphenicol (Cm), 80 μg/ml spectinomycin (Spec), 100 μg/ml ampicillin (Amp), and 100 μg/ml trimethoprim (Tmp). When the optical density at 600 nm (OD₆₀₀) reached approximately 1.4, 0.1 mM of isopropyl-β-D-thiogalactoside (IPTG) and 0.2% (w/v) L-arabinose inducers were added. Induction was carried out at 30° C. for 18 hours. For expression and glycosylation of full-length IgGs, cultures were grown overnight and subsequently subcultured at 37° C. in terrific broth (TB) supplemented with the necessary antibiotics. When the OD₆₀₀ reached approximately 1.4, 0.3 mM of IPTG and 0.2% (w/v) L-arabinose inducers were added. Induction was carried out at 30° C. for 12 hours.

Plasmids for expressing different bacterial OSTs were constructed similarly to pMAF10 (Feldman et al., “Engineering N-linked Protein Glycosylation with Diverse O Antigen Lipopolysaccharide Structures in Escherichia coli,” Proc. Natl. Acad. Sci. USA 102(8):3016-3021 (2005), which is hereby incorporated by reference in its entirety), which encodes CjPglB. Specifically, each of the 24 bacterial OST genes was separately cloned into the EcoRI site of plasmid pMLBAD (Lefebre and Valvano, “Construction and Evaluation of Plasmid Vectors Optimized for Constitutive and Regulated Gene Expression in Burkholderia cepacian Complex Isolates,” Appl. Environ. Microbiol. 68(12):5956-5964 (2002), which is hereby incorporated by reference in its entirety). Template DNA for bacterial OSTs was codon optimized and obtained from Integrated DNA Technologies (IDT). Plasmid pMAF10-CmPglB^{mut} was constructed previously by performing site-directed mutagenesis on CjPglB in pMAF10 to introduce two mutations, D54N and E316Q, that abolish catalytic activity (Ollis et al., “Engineered Oligosaccharyltransferases with Greatly Relaxed Acceptor-Site Specificity,” Nat. Chem. Biol. 10(10):816-822 (2014), which is hereby incorporated by reference in its entirety). Plasmid pMAF10-DmPglB^{mut} was constructed in a similar fashion by introducing analogous mutations, namely D55N and E363Q, to DmPglB in plasmid pMAF10-DmPglB. For purification of DmPglB, plasmid pSF-DmPglB-10xHis was created by replacing the gene encoding CjPglB in plasmid pSF-CjPglB (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety) with the gene encoding DmPglB along with an additional 10xHis sequence using Gibson assembly. For heterologous biosynthesis of the GalNAc₅(Glc)GlcNAc glycan, plasmid pMW07-pglΔBCDEF was generated by deleting the pglCDEF genes coding for biosynthesis of bacillosamine from the pgl locus in plasmid pMW07-pglΔB (Ollis et al., “Engineered Oligosaccharyltransferases with Greatly Relaxed Acceptor-Site Specificity,” Nat. Chem. Biol. 10(10):816-822 (2014), which is hereby incorporated by reference in its entirety) using Gibson assembly cloning. For biosynthesis of the linear GalNAc₅GlcNAc glycan, plasmid pMW07-pglΔBICDEF was generated by additionally deleting the gene coding for the transfer of the branching glucose (pglI). The gene deletions were confirmed by Oxford nanopore whole plasmid sequencing at Plasmidsaurus. For acceptor protein expression, plasmids pBS-scFv13-R4^{DQNAT} pBS-scFv13-R4^{XQNA}T, and pBS-scFv13-R4^{AQNAT-GKG-His}^{₆} were used and are described elsewhere (Ollis et al., “Substitute Sweeteners: Diverse Bacterial Oligosaccharyltransferases with Unique N-Glycosylation Site Preferences,” Sci. Rep. 5:15237 (2015) and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety). Plasmid pBS-scFv13-R4^{QYNST-GKG-His}^{₆} was created by replacing the AQNAT (SEQ ID NO: 22) motif in pBS-scFv13-R4^{AQNAT-GKG-His}^{₆} with QYNST (SEQ ID NO: 5). Plasmid pTrc99S-YcbF-Im7^{DQNAT} described in previous studies (Li et al., “Shotgun Scanning Glycomutagenesis: A Simple and Efficient Strategy Constructing and Characterizing Neoglycoproteins,” Proc. Natl. Acad. Sci. USA 118(39):e2107440118 (2021), which is hereby incorporated by reference in its entirety) was used as a template to create pTrc99S-YebF-Im7^{XXNXT} using degenerate primers with NNK bases (N=A, C, T, or G; K=G or T) at the −2, −1, and +1 positions of the glycosylation sequon. The resulting plasmid DNA library was used to transform DH5α cells as discussed below. Plasmid pTrc99S-spDsbA-hinge-Fc was created by adding the hinge sequence EPKSCDKTHTCPPCP between the E. coli DsbA signal peptide and the human IgG1 Fc domain in pTrc-spDsbA-Fc (Fisher et al., “Production of Secretory and Extracellular N-Linked Glycoproteins in Escherichia Coli,” Appl. Environ. Microbiol. 77(3):871-881 (2011), which is hereby incorporated by reference in its entirety). Plasmid pMAZ360-YMF10-IgG (Mazor et al., “Isolation of Engineered, Full-Length Antibodies from Libraries Expressed in Escherichia coli,” Nat. Biotechnol. 25(5):563-565 (2007), which is hereby incorporated by reference in its entirety) was provided as a gift. All PCRs were performed using Phusion high-fidelity polymerase (New England Biolabs), and the PCR products were gel-purified from the product mixtures to eliminate nonspecific PCR products. The resulting PCR products were assembled using Gibson Assembly Master Mix (New England Biolabs). After transformation of DH5α cells, all plasmids were isolated using a QIAprep Spin Miniprep Kit (Qiagen) and confirmed by DNA sequencing at the Genomics Facility of the Cornell Biotechnology Resource Center.

### GlycoSNAP Assay

Screening of the pTrc99S-YebF-Im7^{XXNXT} library was performed using the glycoSNAP assay (Ollis et al., “Substitute Sweeteners: Diverse Bacterial Oligosaccharyltransferases with Unique N-Glycosylation Site Preferences,” Sci. Rep. 5:15237 (2015); Li et al., “Shotgun Scanning Glycomutagenesis: A Simple and Efficient Strategy Constructing and Characterizing Neoglycoproteins,” Proc. Natl. Acad. Sci. USA 118(39):e2107440118 (2021); and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety). Briefly, E. coli strain CLM24 carrying plasmid pMW07-pglΔBCDEF and pMLBAD encoding the DmPglB OST was transformed with the pTrc99S-YebF-Im7^{XXNXT} library plasmids, yielding a cell library of approximately 1.1×10⁵ members. The resulting transformants were grown on 150-mm LB-agar plates containing 20 μg/mL Cm, 100 μg/mL Tmp, and 80 μg/mL Spec overnight at 37° C. The second day, nitrocellulose transfer membranes were cut to fit 150-mm plates and prewet with sterile phosphate-buffered saline (PBS) before placement onto LB-agar plates containing 20 μg/mL Cm, 100 μg/mL Tmp, 80 μg/mL Spec, 0.1 mM IPTG, and 0.2% (w/v) L-arabinose. Library transformants were replicated onto a nitrocellulose transfer membrane (BioRad, 0.45 μm), which were then placed colony-side-up on a second nitrocellulose transfer membrane and incubated at 30° C. for 18 hours. The nitrocellulose transfer membranes were washed in Tris-buffered saline (TBS) for 10 minutes, blocked in 5% bovine serum albumin for 30 minutes, and probed for 1 hour with fluorescein-labeled SBA (Vector Laboratories, Cat #FL-1011) and Alexa Fluor 647 (AF647)-conjugated anti-His antibody (R&D Systems, Cat #IC0501R) following the manufacturer's instructions. All positive hits were re-streaked onto fresh LB-agar plates containing 20 μg/mL Cm, 100 μg/mL Tmp, and 80 μg/mL Spec and grown overnight at 37° C. Individual colonies were grown in liquid culture to confirm glycosylation of periplasmic fractions, and the sequence of the glycosylation tag was confirmed by DNA sequencing.

### Protein Isolation

To analyze the products of in vivo glycosylation, periplasmic extracts were derived from E. coli cultures as follows. Following induction, cells were harvested by centrifugation at 8,000 rpm for 2 minutes, after which the pellets were resuspended in an amount of 0.4 M arginine such that OD₆₀₀ values were normalized to 10. Following incubation at 4° C. for 1 hour, the samples were centrifuged at 13,200 rpm for 1 minute and the supernatant containing periplasmic extracts was collected. For purification of proteins containing a polyhistidine (6x-His) tag, cells were harvested after induction by centrifugation at 9,000 rpm at 4° C. for 25 minutes and the pellets were resuspended in desalting buffer (50 mM NaH₂PO₄ and 300 mM NaCl) followed by cell lysis using a Emulsiflex C5 homogenizer (Avestin) at 16,000-18,000 psi. The resulting lysate was centrifugated at 9,000 rpm at 4° C. for 25 minutes. The imidazole concentration of the resulting supernatant was adjusted to 10 mM by addition of desalting buffer containing 1 M imidazole. The supernatant was incubated at 4° C. for 1 hour with HisPur Ni-NTA resin (ThermoFisher), after which the samples were applied twice to a gravity flow column at room temperature. The column was washed using desalting buffer containing 10 mM imidazole and proteins were eluted in 2 mL of desalting buffer containing 300 mM imidazole. The eluted proteins were desalted using Zeba Spin Desalting Columns (ThermoFisher) and stored at 4° C.

For protein A purification, harvested cells were resuspended in equilibration buffer (100 mM Na₂HPO₄, 136 mM NaCl, pH 8), followed by cell lysis using a Emulsiflex C5 homogenizer (Avestin) at 16,000-18,000 psi. The resulting lysate was centrifugated at 9,000 rpm at 4° C. for 25 minutes. The supernatant was mixed with the equilibration buffer in a 1:1 ratio by mass, after which the samples were applied to a gravity flow column which contained MabSelect SuRe protein A resin (Cytiva). The column was washed using equilibration buffer. Proteins were eluted using 1 mL of elution buffer (165 mM glycine, pH 2.2). The eluted proteins were collected in a tube containing 100 μL of neutralizing buffer. The eluted fractions were subject to buffer exchange with PBS twice using a 10K MWCO protein concentrator (ThermoFisher). During buffer exchange, samples were centrifugated at 4500 rpm at 4° C. for 20 minutes.

For purification of CjPglB and DmPglB from E. coli, a single colony of BL21DE3 carrying plasmid pSN18 (Kowarik et al., “N-Linked Glycosylation of Folded Proteins by the Bacterial Oligosaccharyltransferase,” Science 314 (5802): 1148-1150 (2006), which is hereby incorporated by reference in its entirety) or pSF-DmPglB-10xHis, respectively, was grown overnight at 37° C. in 20 mL of LB supplemented with Amp. Overnight cells were subcultured into 1 of TB supplemented with Amp and grown until the OD₆₀₀ reached a value of approximately 0.8. The incubation temperature was adjusted to 16° C., after which protein expression was induced by the addition of L-arabinose to a final concentration of 0.02% (w/v). Protein expression was allowed to proceed for 16 hours at 16° C. Cells were harvested by centrifugation, resuspended in 10 mL Buffer A (50 mM HEPES, 250 mM NaCl, pH 7.4) per gram of pellet and then lysed using a homogenizer (Avestin C5 EmulsiFlex). The lysate was centrifuged to remove cell debris, and the supernatant was ultracentrifuged (38,000 rpm; Beckman 70Ti rotor) for 2 hours at 4° C. The resulting pellet containing the membrane fraction was partially resuspended in 25 mL Buffer B (50 mM HEPES, 250 mM NaCl, and 1% (w/v%) n-dodecyl-β-D-maltoside (DDM), pH 7.4). The suspension was incubated at room temperature rotating for 1 hour and then ultracentrifuged (38,000 rpm; Beckman 70Ti rotor) for 1 hour at 4° C. The supernatant containing DDM-solubilized DmPglB was mixed with 0.8 mL of HisPur Ni-NTA resin (ThermoFisher) equilibrated with Buffer B supplemented with protease inhibitor cocktail and incubated rotating for 24 hours at 4° C. After incubation, the material was transferred to a gravity column, washed with Buffer C (50 mM HEPES, 250 mM NaCl, 15 mM imidazole and 1% (w/v) DDM, pH 7.4), and eluted using Buffer D (50 mM HEPES, 250 mM NaCl, 250 mM imidazole and 1% (w/v) DDM, pH 7.4). Purified proteins were stored at a final concentration of 3 mg/mL in a modified OST storage buffer (50 mM HEPES, 250 mM NaCl, 33% (v/v) glycerol, 1% (w/v) DDM, pH 7.5) at −20° C.

For size exclusion chromatography (SEC), purified CjPglB or DmPglB proteins were concentrated to approximately 600 μL using Pierce Protein Concentrator, PES, 10K MWCO (5-20 mL, ThermoFisher). The resulting protein was filter sterilized and further purified using Superdex 200 SEC column (Cytiva) with a buffer containing 50 mM HEPES, 250 mM NaCl, 1% DDM, pH 7.4. The peak fractions were collected and analyzed using Coomassic Brilliant Blue R-250 Staining Solution (Bio-Rad). All fractions containing PglB were concentrated using Pierce Protein Concentrator, PES, 10K MWCO (5 mL, ThermoFisher).

### Immunoblotting

Protein samples (either periplasmic fractions or purified proteins) were solubilized in 10% β-mercaptoethanol (BME) in 4× lithium dodecyl sulfate (LDS) sample buffer and resolved on Bolt Bis-Tris Plus gels (ThermoFisher). The samples were later transferred to immobilon PVDF transfer membranes and blocked with 5% milk (w/v) or 5% bovine serum albumin (w/v) in tris-buffered saline supplemented with 0.1% (w/v) Tween 20 (TBST). The following antibodies were used for immunoblotting: polyhistidine (6x-His) tag-specific polyclonal antibody (1:5000 dilution; Abcam, Cat #ab1187); F(ab′)2-goat anti-human IgG (H+L) secondary antibody conjugated to horseradish peroxidase (HRP) (1:5000 dilution; ThermoFisher, Cat #A24464), C. jejuni heptasaccharide glycan-specific antiserum hR6 (1:1000 dilution; kind gift of Marcus Acbi, ETH Zürich) (Schwarz et al., “Relaxed Acceptor Site Specificity of Bacterial Oligosaccharyltransferase In Vivo,” Glycobiology 21(1):45-54 (2011), which is hereby incorporated by reference in its entirety), and donkey anti-rabbit IgG conjugated to HRP (1:5000 dilution; Cat #ab7083). After probing with primary and second antibodies, the membranes were washed three times with TBST for 10 minutes and subsequently visualized using a ChemiDoc™ MP Imaging System (Bio-Rad). Glycosylation efficiency was determined by performing densitometry analysis of protein bands in anti-His immunoblots using ImageJ software (Schneider et al., “NIH Image to ImageJ: 25 Years of Image Analysis,” Nat. Methods 9(7):671-675 (2012), which is hereby incorporated by reference in its entirety). Briefly, bands corresponding to g0 in each lane were grouped as a row or a horizontal “lane” and quantified using the gel analysis function in ImageJ. The bands corresponding to g1 were analyzed identically. The resulting intensity data for g0 and g1 was used to calculate percent glycosylated expressed according to the following ratio: g1/[g0+g1]. Efficiency data was calculated from immunoblots corresponding to three biological replicates, with all data were reported as the mean±SD. Statistical significance was determined by paired Student's 1 tests (*p<0.05, **p<0.01; ***p<0.001; ****p<0.0001) using Prism 10 for MacOS version 10.3.0.

### Glycoproteomic Tandem MS Analysis

Purified proteins were reduced by heating in 25 mM DL-dithiothreitol (DTT) at 50° C. for 45 minutes, then cooled down to room temperature, immediately alkylated by incubating with 90 mM iodoacetamide (IAA) at room temperature in dark for 20 minutes. Samples were loaded on the top of 10-kDa molecular weight cut-off (MWCO) filters (MilliporeSigma), desalted by passing through with 800 μL 50 mM ammonium bicarbonate (Ambic). Proteins were recovered from the filters and reconstituted as 1 μg/μL solution in 50 mM Ambic. Sequencing grade trypsin (Promega) was added to samples at a 1:20 ratio, digestion was performed at 37° C. overnight. Trypsin activity was terminated by heating at 100°° C. for 5 minutes. Cooled samples were reconstituted in LC-MS grade 0.1% formic acid (FA) as 0.1 μg/μL solution, passed through 0.2 μm filters (Fisher Scientific). LC-MS/MS was carried out on an Ultimate 3000 RSLCnano low-flow liquid chromatography system coupled with Orbitrap Tribrid Eclipse mass spectrometer via a Nanospray Flex ion source. Samples were trap-loaded on a 2 μm pore size 75 μm×150 mm Acclaim PepMap 100 C18 nanoLC column. The column was equilibrated at 0.300 μL/min flowrate with 96% Buffer A (0.1% FA) and 4% Buffer B (80% acetonitrile (ACN) with 0.1% FA). A 60-minutes gradient in which Buffer B ramped from 4% to 62.5% was used for peptide separation. To scrutinize the expected glycan attachment at the anticipated sequon, a higher collision energy dissociation (HCD) product triggered collision induced dissociation (CID) (HCDpdCID) MS/MS fragmentation cycle in 3-s frame was used. Precursors were scanned in Orbitrap at 120,000 resolution and fragments were detected in Orbitrap at 30,000 resolution (Shajahan et al., “Deducing the N- and O-glycosylation profile of the spike protein of novel coronavirus SARS-COV-2,” Glycobiology 30(12):981-988 (2020), which is hereby incorporated by reference in its entirety).

LC-MS/MS data was searched in Byonic (v5.0.3) and manually inspected in Freestyle (v1.8 SP1). For IgG-Fc and full-length IgG analysis, IgG sequences with fully reversed decoy were used for peptide backbone identification. The precursor mass tolerance was set at 5 ppm, while the fragment mass tolerance was allowed as 20 ppm. Expected glycan composition HexNAc(6) or HexNAc(6)Hex(1) based on the specific glycosylation pathway was registered in N-glycan list. Protein list output was set with a cutoff at 1% FDR (false detection rate) or 20 reverse sequences, whichever came last. Only fully specific trypsin-cleaved peptides with up to 2 mis-cleavages were considered. Carbamidomethylation on cysteine was considered as fixed modification. Oxidation on methionine, deamidation on asparagine and glutamine were considered as variable modifications. Peptide identity and modifications were annotated by Byonic, followed by manual inspection of peptide backbone b/y ions, glycan oxonium ions, and glycopeptide neutral losses (Lee et al., “Toward Automated N-Glycopeptide Identification in Glycoproteomics,” J. Proteome Res. 15(10):3904-3915 (2016), which is hereby incorporated by reference in its entirety). Relative abundance of glycoforms reported were based on area under the curve of deconvoluted extracted ion chromatogram (XIC) peaks processed in Freestyle using the protein Averagine model. Aglycosylated QYNST (SEQ ID NO: 5) peptide XIC in the same run was used for relative quantification. Accurate precursor masses and retention times were used as additional identification bases, when the fragments of either glycopeptide or aglycosylated peptide in a pair, but not both, were suppressed in LC23 MS/MS acquisition (Klein and Zaia, “Relative Retention Time Estimation Improves N-Glycopeptide Identifications by LC-MS/MS,” J. Proteome Res. 19(5):2113-2121 (2020), which is hereby incorporated by reference in its entirety). To confidentially locate N-glycosylation sites on and covalent glycan attachment to scFv13-R4(N34L/N77L)^{QYNST} and DmPglB, sequential trypsin/a-lytic protease digestion was performed at a 1:20 ratio. A stepped collision energy HCD product-triggered electron transfer dissociation with assisted HCD (EThcD) (stepped HCDpdEThcD) MS/MS program was used. Confident N-glycosylation site mapping on these two samples required a/b/c/y/z fragment ions retaining glycosylation delta mass. Quantitative information from the complicated glycosylation states of DmPglB was not gathered.

### In Vitro Glycosylation

For in vitro glycosylation of DmPglB, 500 μL of in vitro glycosylation buffer (10 mM HEPES, pH 7.5, 10 mM MnCl₂, and 0.1% (w/v) DDM) containing 50 μg of purified DmPglB and 50 μL of solvent extracted LLOs were incubated at 30° C. for 16 hours. Organic solvent extraction of LLOs bearing the GalNAc₅(Glc)GlcNAc glycan from the membrane of E. coli cells was performed as follows. A single colony of CLM24 carrying the plasmid pMW07-pglΔBICDEF was inoculated in LB supplemented with Cm and grown overnight at 37° C. Overnight cells were then subcultured into 1 L of TB supplemented with Cm and grown until the OD₆₀₀ reached approximately 0.8. The incubation temperature was adjusted to 30° C. and expression induced with 0.2% (w/v) L-arabinose. After 16 hours, cells were harvested by centrifugation, resuspended in 50 mL MeOH, and dried overnight. The next day, dried cell material was scraped into a 50-mL conical tube and pulverized. The pulverized material was then thoroughly mixed with 12 mL of 2:1 mixture of chloroform:methanol, sonicated in a water bath for 10 minutes, centrifuged at 4,000 rpm and 4° C. for 10 minutes, and the supernatant discarded. This step was then repeated two more times. Subsequently, 20 mL of water was thoroughly mixed with the pellet, sonicated in a water bath for 10 minutes, centrifuged at 4,000 rpm and 4° C. for 10 minutes, and the supernatant discarded. The pellet was vortexed with 18 mL of a 10:10:3 mixture of chloroform:methanol:water and sonicated in a water bath to homogeneity. 8 mL of methanol was subsequently added, the mixture was vortexed, and then centrifuged at 4,000 rpm and 4° C. for 10 minutes. The supernatant was decanted and retained while the pellet discarded. Then, 8 mL of chloroform and 2 mL of water were added to the supernatant, mixed, and centrifuged at 4,000 rpm and 4° C. for 10 minutes. The aqueous supernatant was aspirated and discarded, while the organic bottom layer containing the LLO was dried overnight. The next day, dried material was resuspended in cell-free glycosylation buffer (10 mM HEPES, pH 7.5, and 0.1% (w/v) DDM) and stored at −20 C.

In vitro glycosylation was also performed using fluorescently labeled acceptor peptides. For turnover rate measurements, each reaction was prepared in a total volume of 80 μL containing: 8 μL of in vitro glycosylation buffer (500 mM HEPES, 1% (w/v) DDM), 1.6 μL of 1 M MnCl₂, 0.18 μM of purified PglB, 16 μL of solvent-extracted LLOs bearing the GalNAc₅(Glc)GlcNAc structure, 0.5 μM of fluorescently labeled acceptor peptide TAMRA-GSDQNATF-NH₂ (SEQ ID NO: 65) or TAMRA-GQYNSTAF-NH₂ (SEQ ID NO: 66)) (GenScript) and 32 μL of ddH₂O. Reactions were incubated in a water bath at 30° C., with samples collected at different time points. Reactions were stopped by boiling the sample at 90° C. for 5 minutes. For Michaelis-Menten kinetics, reactions were performed in a total volume of 10 μL containing: 1 μL of in vitro glycosylation buffer, 0.2 μL of 1 M MnCl₂, 0.18 μM of purified PglB, 2 μL of solvent-extracted LLOs bearing the GalNAc₅(Glc)GlcNAc structure, varying concentrations of fluorescently labeled acceptor peptide (ranging from 0.25 to 30 μM), and ddH2O as needed. The reactions were incubated for 18 h at 30°° C. and stopped by boiling the sample at 90° C. for 5 minutes.

### In-Gel Fluorescence Detection

Samples were diluted 1:6 with Novex Tricine SDS Running Buffer (1×). Each sample was then mixed with dye that was produced in-house and boiled at 80° C. for 2 minutes. The dye consisted of 200 mM Tris-Cl (pH 6.8), 8% (w/v) sodium dodecyl sulfate (SDS; electrophoresis grade), and 40% (v/v) glycerol. For Michaelis-Menten kinetics, the samples were normalized to a final concentration of 0.25 μM. A total of 8 μL of each sample was loaded onto Novex 16% Tricine Mini Protein Gels (1.0 mm thickness). The Spectra™ Multicolor Low Range Protein Ladder was used as the molecular weight marker. The gel was run at 70 V for 2.5 hours at 4° C. and subsequently imaged using a ChemiDoc MP Imaging System (Bio-Rad). DyLight 550 was used to visualize the fluorescently labeled peptides, while the Spectra ladder was visualized using Cy5.5.

### Chemoenzymatic Glycan Remodeling

A total of 400 U of exo-α-N-acetylgalactosaminidase (New England Biolabs, Cat # P0734S) was added to a solution of GalNAc₅GlcNAc-hinge-Fc dimer (200 g) in 100 μL GlycoBuffer 1 (50 mM NaOAc, 5 mM CaCl₂, pH 5.5) and the reaction mixture was incubated at room temperature. Reaction progress was monitored by LC-ESI-MS using an Exactive Plus Orbitrap Mass Spectrometer (Thermo Scientific) equipped with an Agilent Poroshell 300SB C8 column (5 μm, 1.0×75 mm) and was found to be complete after just 2 hours. The sample was then buffer exchanged to 100 mM Tris pH 7 buffer using an Amicon® Ultra 0.5 mL 10K Centrifugal Filter (Millipore) and concentrated to 2 mg/mL. To this solution was added G2-oxazoline (320 μg, 30 mol eq), followed by 1 μg of EndoS2-D184M to a final concentration of 0.4% (w/w) relative to the hinge-Fc. The sample was incubated at 30° C., and the reaction monitored by LC-ESI-MS. After 30 minutes, the reaction was complete, and the G2-hinge-Fc product was purified using a 1-mL Protein A HP column (Cytiva) following previously established procedures (Li et al., “Modulating IgG Effector Function by Fc Glycan Engineering,” Proc. Natl. Acad. Sci. USA 114(13):3485-3490 (2017), which is hereby incorporated by reference in its entirety). The final product was buffer exchanged to PBS by centrifugal filtration and stored at −80° C. until later use.

### ELISA

For binding assays between IgG-Fc domain and Fcγ receptor, FcγRIIIA V158 (10 μg/mL; Sino Biological) in PBS buffer (pH 7.4) was coated onto a high-binding 96-well plate (VWR) overnight at 4°° C. After washing with PBST (PBS, 0.1% Tween 20) the plate was blocked overnight at 4° C. with 200 μL of 5% milk (w/v) in PBST. The plate was washed three times and 100-μL serial dilutions of sample were added to each well. The concentrations of each glycosylated and aglycosylated sample ranged from 0.08 to 10 μg/mL (fivefold serial dilutions). All IgG-Fc glycoforms were purified proteins except for commercial trastuzumab (HY-P9907, MedChem Express). The plate was placed on a shaker and incubated for 1 hour at 37° C. After incubation, the plate was washed three times and incubated for 1 hour with 100 μL of F(ab′)2-goat anti-human IgG (H+L) antibody conjugated to HRP (1:5,000 dilution; ThermoFisher, Cat #A24464). After three washes, 100 μL of 3,3′,5,5′ tetramethylbenzidine (TMB) ELISA substrate (ThermoFisher) were added to each well for signal development. The reaction was stopped upon addition of 100 μL of 2M sulfuric acid. The absorbance of samples was measured at 450 nm using a SpectraMax 190 microplate reader (Molecular Devices) and the data was analyzed using GraphPad Prism software (version 10.0.2) by nonlinear regression analysis.

### Sequence Alignments and Structural Models

Sequences were aligned using the Clustal Omega web server (Madeira et al., “Search and Sequence Analysis Tools Services from EMBL-EBI in 2022,” Nucleic Acids Res 50:W276-W279 (2022), which is hereby incorporated by reference in its entirety). The structure of C. lari PglB was derived from the PDB entry 5OGL (Lizak et al., “X-Ray Structure of a Bacterial Oligosaccharyltransferase,” Nature 474(7351):350-355 (2011), which is hereby incorporated by reference in its entirety). Structures for all other OSTs were obtained with the AlphaFold2 (AF2) protein structure prediction algorithm implemented with ColabFold (Mirdita et al., “ColabFold: Making Protein Folding Accessible to All,” Nat. Methods 19(6):679-682 (2022) and Jumper et al., “Highly Accurate Protein Structure Prediction with AlphaFold,” Nature 596(7873):583-589 (2021), which are hereby incorporated by reference in their entirety). All structures were generated with standard settings, 8 recycles and relaxed with Amber. Two sets of structures were generated—one with and one without the substrate peptide GGQYNST. However, AF2 failed to place the peptide in the peptide binding pocket of the enzyme for all enzymes. In these cases, the structure of enzyme-peptide complexes was obtained by manually aligning the enzyme structures from AF2 to the enzyme-peptide complex (with DQNAT (SEQ ID NO: 6) peptide) for the ClPglB crystal structure from PDB entry 5OGL Lizak et al., “X-Ray Structure of a Bacterial Oligosaccharyltransferase,” Nature 474(7351):350-355 (2011), which is hereby incorporated by reference in its entirety). To model the QYNST (SEQ ID NO: 5) peptide in the peptide-binding pocket, the DQNAT (SEQ ID NO: 6) peptide was mutated to QYNST (SEQ ID NO: 5) and the QYNST (SEQ ID NO: 5) peptide in the peptide-binding pocket of each enzyme's AF2 model was relaxed with Rosetta's relax function. Twenty-five structures were generated using the Rosetta relax function with default parameters for each enzyme-peptide complex and the structure with the lowest total score was selected. Electrostatic surfaces were generated based on electrostatics calculations using the APBS plugin in PyMOL, which combines standard focusing techniques and the Bank-Holst algorithm into a “parallel focusing” method for the solution of the Poisson-Boltzmann equation (PBE) for nanoscale systems (Baker et al., “Electrostatics of Nanosystems: Application to Microtubules and the Ribosome,” Proc. Natl. Acad. Sci. USA 98:10037-10041 (2001), which is hereby incorporated by reference in its entirety).

### Example 1—Bioprospecting of Desulfobacterota for Interesting ssOST Candidates

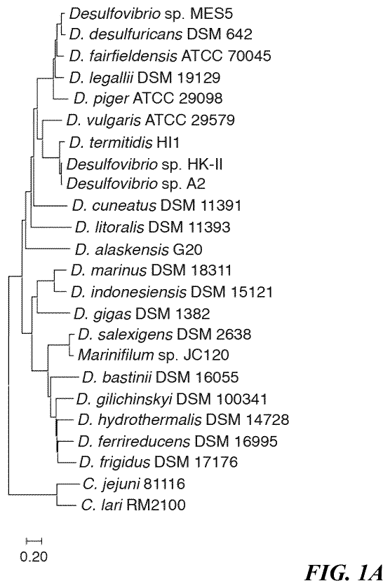

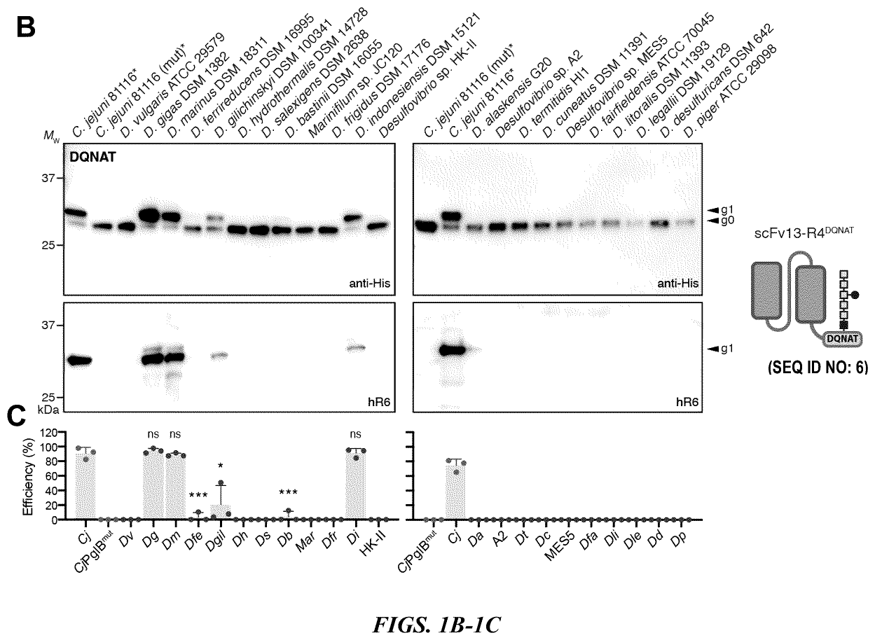

The current armamentarium of characterized bacterial ssOSTs is insufficient for glycoprotein engineering applications that endeavor to recapitulate human-type glycosylation of biotherapeutic proteins (Schwarz et al., “A Combined Method for Producing Homogeneous Glycoproteins with Eukaryotic N-Glycosylation,” Nat. Chem. Biol. 6(4):264-266 (2010); Valderrama-Rincon et al., “An Engineered Eukaryotic Protein Glycosylation Pathway in Escherichia Coli,” Nat. Chem. Biol. 8(5):434-436 (2012); and Glasscock et al., “A Flow Cytometric Approach to Engineering Escherichia coli for Improved Eukaryotic Protein Glycosylation,” Metab. Eng. 47:488-495 (2018), which are hereby incorporated by reference in their entirety). Therefore, novel PglB homologs from Desulfovibrio spp. that have relaxed sequon specificity and catalyze glycosylation of diverse sequons with higher efficiency than previously discovered enzymes were sought out. A collection of 19 candidate OSTs with similarity to DaPglB and DgPglB (FIG. 1A) were identified. DaPglB and DgPglB were chosen as the query sequences because these OSTs previously exhibited the most efficient glycosylation of non-canonical sequences (e.g., AQNAT (SEQ ID NO: 22)) (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety) and thus do not conform to the −2 rule that has been established for CjPglB (Kowarik et al., “Definition of the Bacterial N-Glycosylation Site Consensus Sequence,” EMBO J. 25(9):1957-1966 (2006), which is hereby incorporated by reference in its entirety). For context, DaPglB and DgPglB share 30% identity with each other and only approximately 15-20% with the prototypic bacterial ssOSTs, CjPglB and C. lari PglB (ClPglB). In fact, the catalytic region of Desulfovibrio PglBs, which contains the signature WWDXG (SEQ ID NO: 23) motif that is essential for function and thought to play a key role in catalysis (Yan et al., “Studies on the Function of Oligosaccharyl Transferase Subunits. Stt3p is Directly Involved in the Glycosylation Process,” J. Biol. Chem. 277(49):47692-47700 (2002), which is hereby incorporated by reference in its entirety), is more similar to the catalytic region of eukaryotic and archaeal OSTs than to the same region of CjPglB (Ollis et al., “Substitute Sweeteners: Diverse Bacterial Oligosaccharyltransferases with Unique N-Glycosylation Site Preferences,” Sci. Rep. 5:15237 (2015) and Ielmini and Feldman, “Desulfovibrio desulfuricans PglB Homolog Possesses Oligosaccharyltransferase Activity with Relaxed Glycan Specificity and Distinct Protein Acceptor Sequence Requirements,” Glycobiology 21(6):734-742 (2011), which are hereby incorporated by reference in their entirety). Here, a total of 10 DgPglB homologs were selected, with DmPglB and D. indonesiensis DSM 15121 PglB (DiPglB) exhibiting the highest identity (42% and 47%, respectively) and D. desulfuricans DSM 642 PglB exhibiting the lowest (30%) identity. A further 9 PglB homologs with homology to DaPglB were selected, with Desulfovibrio sp. A2 PglB and D. litoralis DSM 11393 PglB exhibiting the highest (38%) and lowest (30%) identity, respectively.

### Example 2—A Subset of Desulfovibrio PglB Homologs Exhibit Efficient OST Activity

To functionally evaluate the curated list of Desulfobacterota OSTs, an ectopic trans-complementation assay was employed (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety). The assay is based on E. coli strain CLM24, which lacks native glycosylation but is rendered glycosylation competent by transformation with one plasmid encoding enzymes for N-glycan biosynthesis, a second plasmid encoding a candidate PglB homolog, and a third plasmid encoding a glycoprotein target bearing cither an engineered or natural N-glycan acceptor site. Using this assay, candidate PglB homologs were provided in trans and tested for their ability to promote glycosylation activity in E. coli.

To minimize microheterogeneity so that modified acceptor proteins were homogenously glycosylated, plasmid pMW07-pglΔBCDEF was used. This plasmid was previously shown to yield glycoproteins that were predominantly glycosylated (>98%) with GalNAc₅(Glc)GlcNAc, a mimic of the C. jejuni N-glycan but with reducing-end GlcNAc replacing bacillosamine (Li et al., “Shotgun Scanning Glycomutagenesis: A Simple and Efficient Strategy Constructing and Characterizing Neoglycoproteins,” Proc. Natl. Acad. Sci. USA 118(39):e2107440118 (2021), which is hereby incorporated by reference in its entirety). This reducing-end GlcNAc could be further advantageous as a substrate for PglB enzymes from Desulfovibrio spp. given that at least one glycoprotein from D. gigas, the 16-heme cytochrome HmcA, involves the formation of a GlcNAcasparagine linkage at N261 of HmcA (Santos-Silva et al., “Crystal structure of the 16 heme cytochrome from Desulfovibrio gigas: a glycosylated protein in a sulphate-reducing bacterium,” J. Mol. Biol. 370(4):659-673 (2007), which is hereby incorporated by reference in its entirety). Moreover, this linkage also occurs in eukaryotic N-glycoproteins and can be remodeled to create a eukaryotic complex-type glycan via a two-step enzymatic trimming/transglycosylation process (Schwarz et al., “A Combined Method for Producing Homogeneous Glycoproteins with Eukaryotic N-Glycosylation,” Nat. Chem. Biol. 6(4):264-266 (2010), which is hereby incorporated by reference in its entirety). Codon-optimized versions of each Desulfovibrio pglB gene were expressed from plasmid pMLBAD. For the acceptor protein, anti-β-galactosidase single-chain Fv antibody clone 13-R4 (scFv13-R4) fused with an N-terminal co-translational Sec export signal and a C-terminal DQNAT (SEQ ID NO: 6) glycosylation tag (Valderrama-Rincon et al., “An Engineered Eukaryotic Protein Glycosylation Pathway in Escherichia Coli,” Nat. Chem. Biol. 8(5):434-436 (2012), which is hereby incorporated by reference in its entirety) was expressed from plasmid pBS-scFv13-R4^{DQNAT}. scFv13-R4^{DQNAT} was chosen as a model acceptor protein because it is well expressed in the E. coli periplasm and can be efficiently glycosylated by diverse PglB homologs (Ollis et al., “Substitute Sweeteners: Diverse Bacterial Oligosaccharyltransferases with Unique N-Glycosylation Site Preferences,” Sci. Rep. 5:15237 (2015); Valderrama-Rincon et al., “An Engineered Eukaryotic Protein Glycosylation Pathway in Escherichia Coli,” Nat. Chem. Biol. 8(5):434-436 (2012); and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety). It should be noted that DQNAT (SEQ ID NO: 6) is an optimal sequon for CjPglB 32 and has been widely used as a tag for studying PglB-mediated glycosylation in E. coli (Fisher et a., “Production of Secretory and Extracellular N-linked Glycoproteins in Escherichia coli,” Appl. Environ. Microbiol. 77:871-881 (2011), which is hereby incorporated by reference in its entirety).

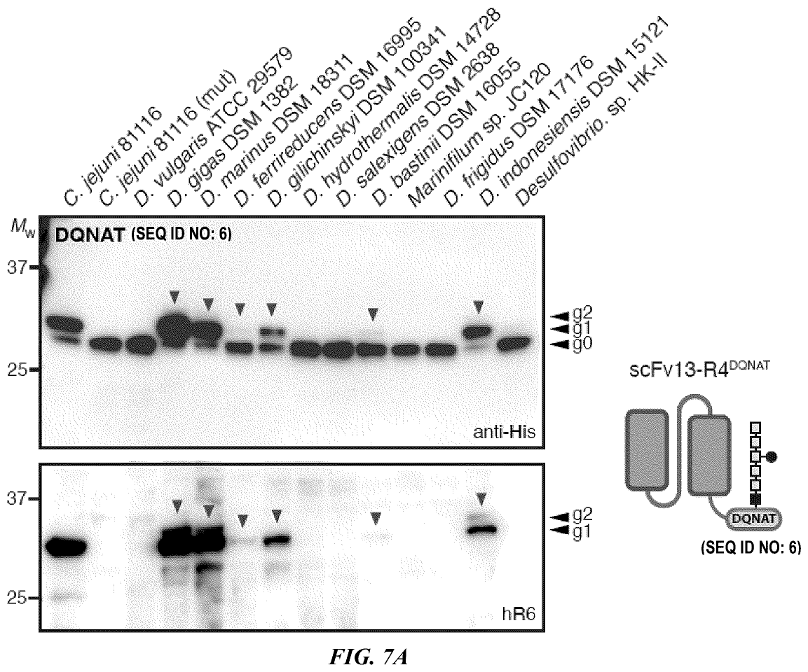

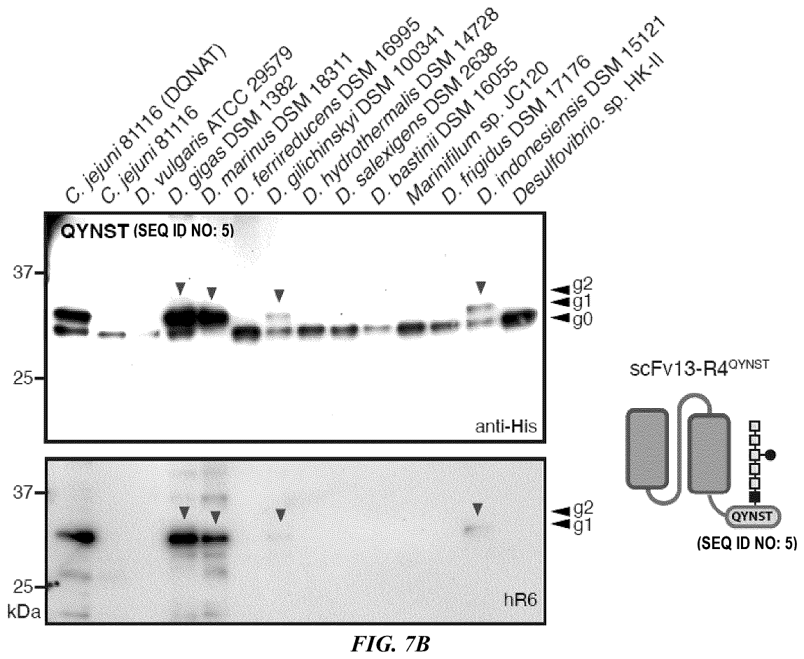

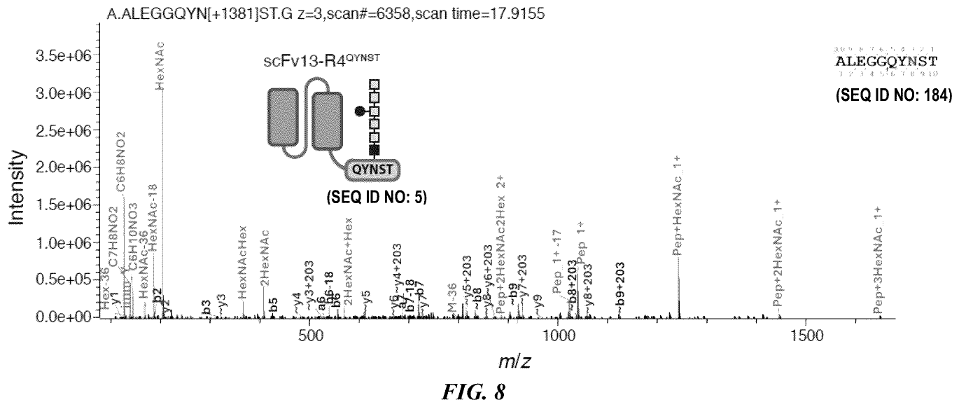

Glycosylation of the periplasmic scFv 13-R4^{DQNAT} protein was evaluated by immunoblot analysis with a polyhistidine epitope tag-specific antibody (anti-His) or C. jejuni heptasaccharide-specific serum (hR6) (Schwarz et al., “Relaxed Acceptor Site Specificity of Bacterial Oligosaccharyltransferase In Vivo,” Glycobiology 21(1):45-54 (2011), which is hereby incorporated by reference in its entirety). As expected, positive control cells complemented with wild-type (wt) CjPglB produced two proteins that were detected with the anti-His antibody, which corresponded to the unglycosylated (g0) and monoglycosylated (g1) forms of scFv13-R4^{DQNAT} (FIG. 1B). Subsequent detection of the higher molecular weight g1 band with hR6 serum specific for the C. jejuni glycan confirmed glycosylation of this protein by wt CjPglB. In contrast, negative control cells complemented with a CjPglB mutant rendered inactive by two active-site mutations, D54N and E316Q (hereafter CjPglB^{mut}), produced only the g0 form of scFv13-R4^{DQNAT} with no detectable signal from the hR6 serum (FIG. 1B), confirming lack of glycosylation in these cells. Of the 22 Desulfobacterota PglB homologs tested here (19 newly curated and 3 that were tested previously, namely DaPglB, DgPglB and DvPglB (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety)), a total of four (DgPglB, DiPglB, DmPglB, and D. gilichinskyi PglB (DgilPglB)) were functionally expressed based on their ability to promote clearly detectable glycosylation of the canonical DQNAT (SEQ ID NO: 6) motif as determined by immunoblot analysis with the anti-His antibody and hR6 serum (FIG. 1B). Of these, DgPglB, DiPglB, and DmPglB showed the highest glycosylation efficiency (all >89% based on densitometry analysis), rivaling that observed for CjPglB (91%) (FIG. 1C). These three highly efficient OSTs also produced an additional slower migrating band in the anti-His and hR6 blots, corresponding to a diglycosylated (g2) form of scFv13-R4^{DQNAT}. This g2 form likely resulted from the glycosylation of a native ⁷⁵RDNAT⁷⁹ (SEQ ID NO: 27) motif in scFv13-R4 that was previously observed to be glycosylated by Desulfovibrio PglB homologs such as DgPglB having relaxed sequon specificity (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety). Two additional enzymes, D. bastini PglB (DbPglB) and D. ferrireducens PglB (DfPglB), showed weak glycosylation that appeared following longer exposure of the same blots (FIG. 7A and FIG. 7B).

### Example 3—DmPglB Efficiently Glycosylates Non-Canonical Sequons

To determine whether any of the Desulfovibrio PglB homologs also recognized sequons with a non-acidic amino acid in the −2 position, glycosylation of the acceptor protein scFv13-R4^{AQNAT}, which carries an AQNAT (SEQ ID NO: 22) motif at its C-terminus, was evaluated. AQNAT (SEQ ID NO: 22) is considered a non-canonical sequon because it is not glycosylated by CjPglB (Kowarik et al., “Definition of the Bacterial N-Glycosylation Site Consensus Sequence,” EMBO J. 25(9):1957-1966 (2006), which is hereby incorporated by reference in its entirety). Hence, the ability to glycosylate AQNAT (SEQ ID NO: 22) and other related sequons in which D/E residues are absent from the −2 position serves as a measuring stick for relaxed substrate specificity (Ollis et al., “Substitute Sweeteners: Diverse Bacterial Oligosaccharyltransferases with Unique N-Glycosylation Site Preferences,” Sci. Rep. 5:15237 (2015); Schwarz et al., “Relaxed Acceptor Site Specificity of Bacterial Oligosaccharyltransferase In Vivo,” Glycobiology 21(1):45-54 (2011); Ielmini and Feldman, “Desulfovibrio desulfuricans PglB Homolog Possesses Oligosaccharyltransferase Activity with Relaxed Glycan Specificity and Distinct Protein Acceptor Sequence Requirements,” Glycobiology 21(6):734-742 (2011); and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety). To eliminate any potential confounding results related to additional sequons, an scFv13-R4 variant in which two putative internal glycosylation sites (³²FSNYS³⁶ (SEQ ID NO: 26) and ⁷⁵RDNAT⁷⁹ (SEQ ID NO: 27)) were mutated by introducing N34L and N77L substitutions was also evaluated. These mutations were previously shown to eliminate the g2 form of this protein arising from glycosylation at position N77 (N34 was not observed to be glycosylated) (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety).

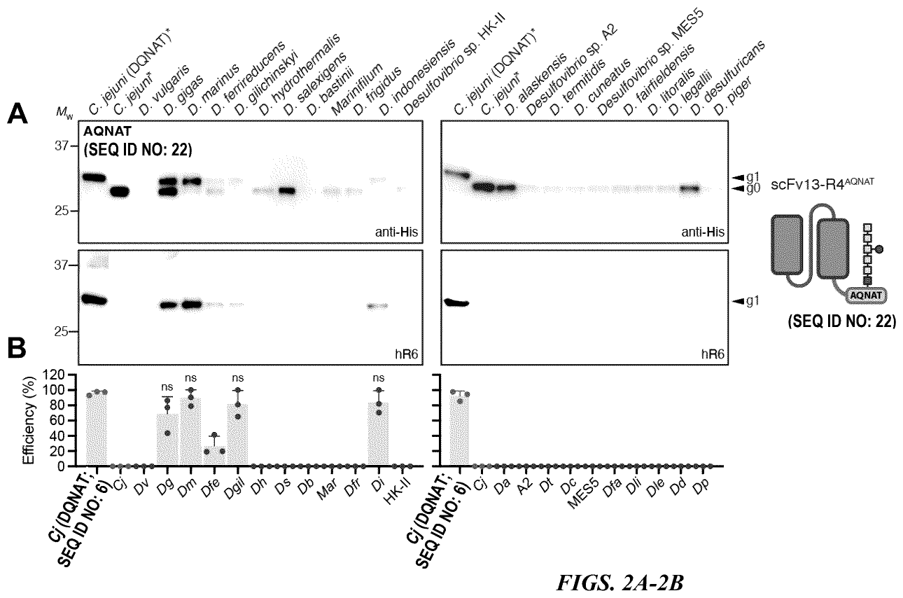

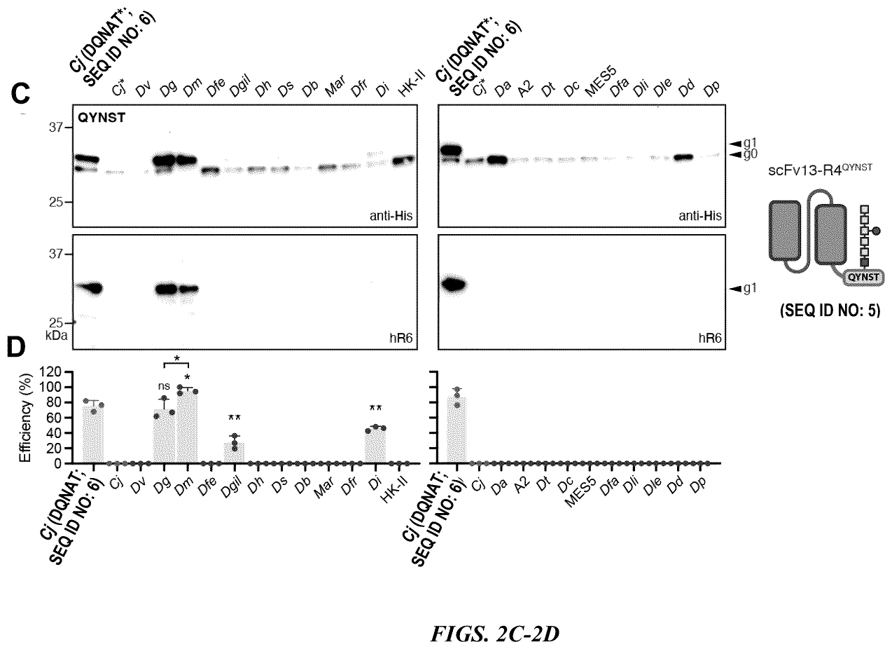

Of the six Desulfobacterota PglB homologs that showed activity towards scFv13-R4^{DQNAT} above, all but DbPglB were capable of glycosylating the scFv13-R4(N34L/N77L)^{AQNAT}construct based on immunoblot analysis with anti-His antibody and hR6 serum (FIG. 2A). In contrast, CjPglB was unable to glycosylate the AQNAT (SEQ ID NO: 22) motif, as expected given its preference for D/E in the −2 position (Kowarik et al., “Definition of the Bacterial N-Glycosylation Site Consensus Sequence,” EMBO J. 25(9):1957-1966 (2006), which is hereby incorporated by reference in its entirety). The remaining OSTs failed to show any measurable activity, which together with their lack of activity above, suggests that they either prefer sequons besides (D/A)QNAT (SEQ ID NO: 28) or were non-functional in our trans-complementation assay for other reasons, such as poor expression or incompatibility with the GalNAc₅(Glc)GlcNAc glycan and/or C-terminal location of the sequon. Importantly, DgPglB, DiPglB, and DmPglB were again among the most active OSTs in terms of glycosylation efficiency, with DmPglB reaching 90% (FIG. 2B). Interestingly, DfPglB and DgilPglB showed significantly stronger glycosylation of scFv13-R4(N34L/N77L)^{AQNAT} versus scFv13-R4^{DQNAT}, suggesting that these enzymes may possess a bias for sequons with non-acidic residues in the −2 position. It should be noted that while DaPglB was previously observed to glycosylate scFv13-R4(N34L/N77L)^{AQNAT} (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety), activity for this OST with the AQNAT (SEQ ID NO: 22) sequon were not detected under the conditions tested.

To further investigate the ability of PglB homologs from Desulfovibrio to recognize non-canonical sequences, glycosylation of the acceptor protein scFv13-R4 (N34L/N77L)^{QYNST}, which carries a QYNST (SEQ ID NO: 5) motif at its C-terminus, was evaluated. QYNST (SEQ ID NO: 5) was chosen because IgG antibodies, one of the most abundant glycoproteins in human scrum, are invariably decorated with N-glycans at a highly conserved QYNST (SEQ ID NO: 5) motif in their Fc region. Whereas scFv13-R4(N34L/N77L)^{QYNST} was not glycosylated by CjPglB, consistent with its restricted sequon specificity (Kowarik et al., “Definition of the Bacterial N-Glycosylation Site Consensus Sequence,” EMBO J. 25(9):1957-1966 (2006), which is hereby incorporated by reference in its entirety), four Desulfovibrio ssOSTs—DgPglB, DmPglB, DiPglB, and DgilPglB—exhibited glycosylation of the non-canonical QYNST (SEQ ID NO: 5) sequon as revealed by immunoblotting (FIG. 2C, FIG. 7A, and FIG. 7B) and mass spectrometry (MS) analysis (FIG. 8; shown for DmPglB). Of these, DmPglB displayed the highest glycosylation efficiency (95%) (FIG. 2D), making it the only OST that was capable of glycosylating all three sequons with approximately 90% efficiency or higher.

### Example 4—DmPglB Exhibits Extremely Relaxed Sequon Specificity

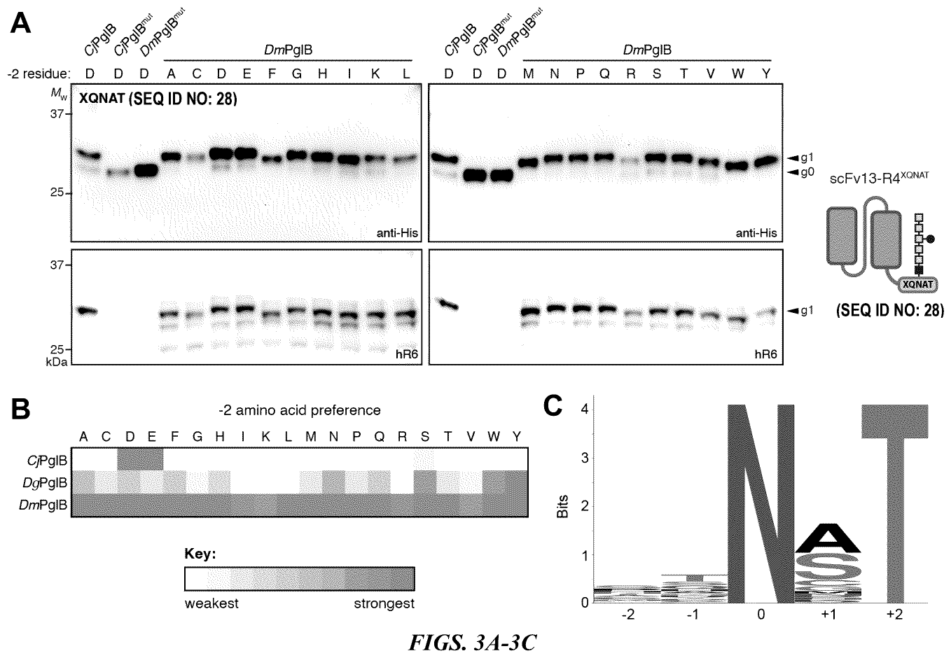

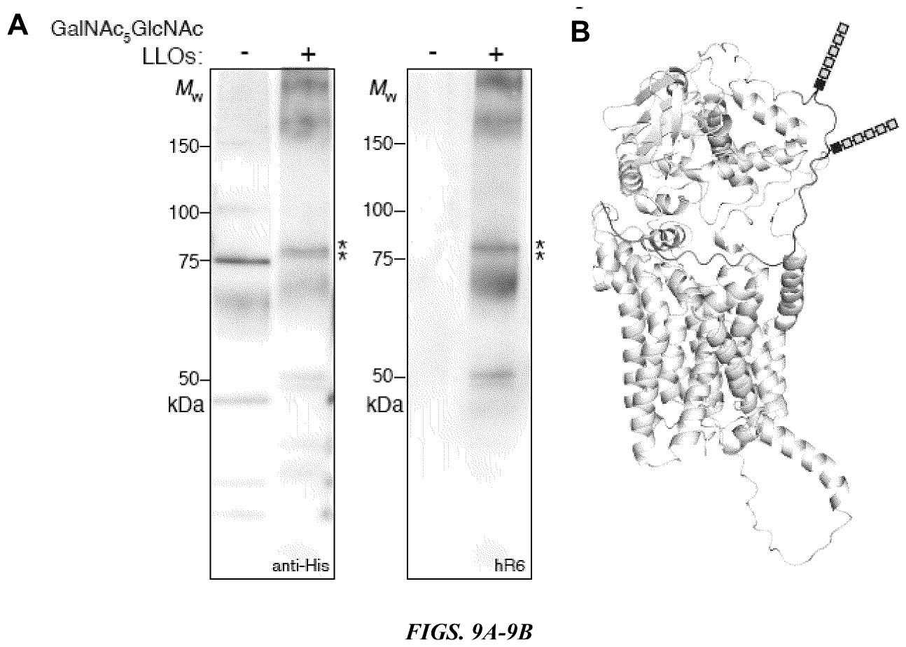

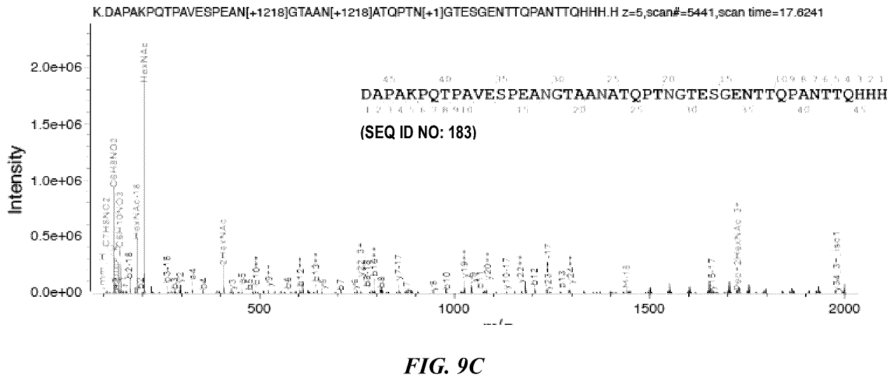

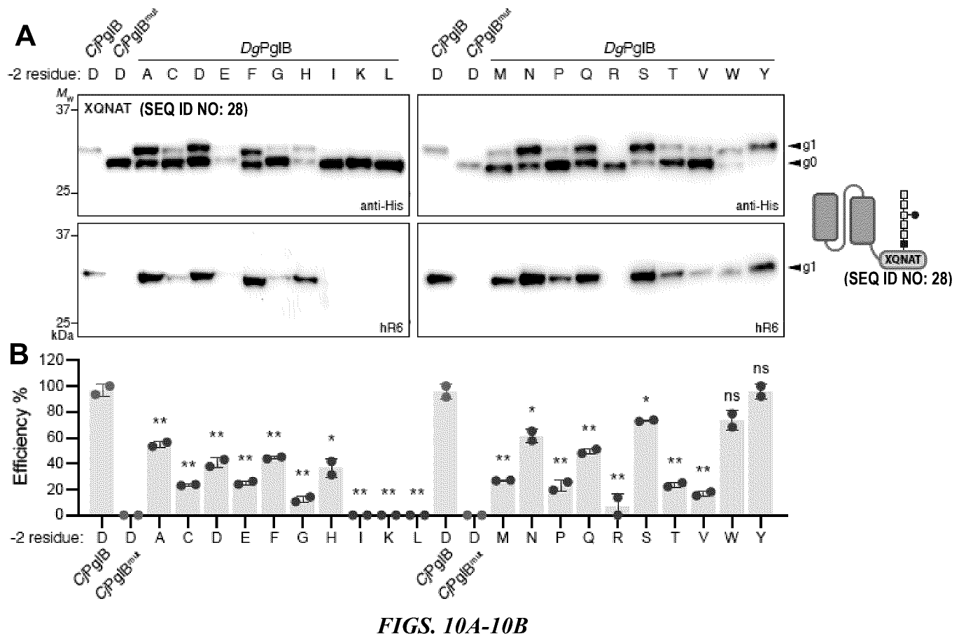

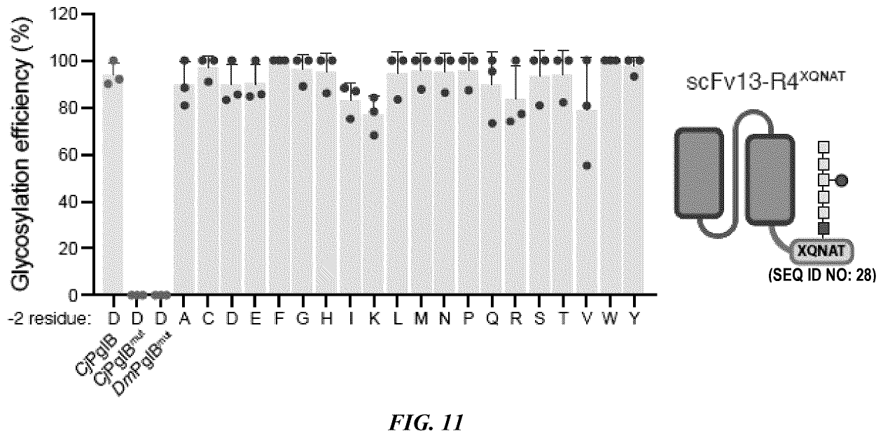

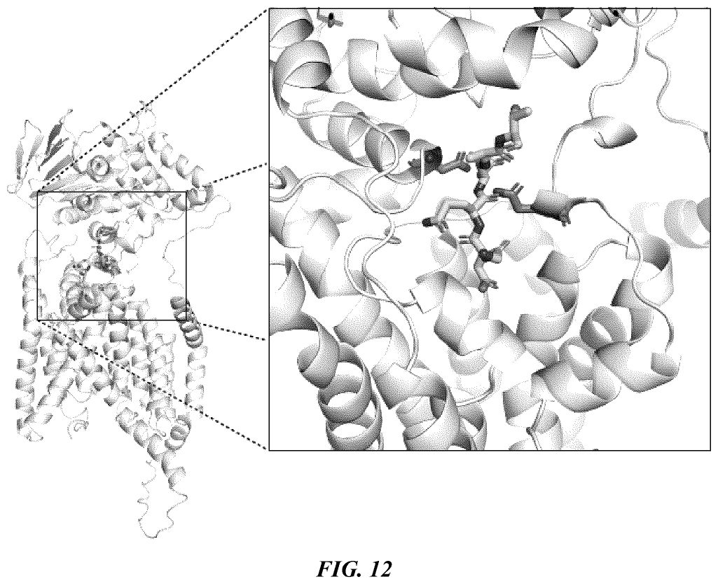

During these experiments, autoglycosylation of DmPglB was observed (FIG. 9A), indicating that DmPglB itself is a glycoprotein, just like CjPglB and ClPglB (Lizak et al., “X-Ray Structure of a Bacterial Oligosaccharyltransferase,” Nature 474(7351):350-355 (2011) and Bokhari et al., “Oligosaccharyltransferase PglB of Campylobacter jejuni is a Glycoprotein,” World J. Microbiol. Biotechnol. 36(1):9 (2019), which are hereby incorporated by reference in their entirety). Specifically, MS analysis identified two sequons clustered at the extreme C-terminus of DmPglB that were autoglycosylated, namely ⁷⁵¹EANGT⁷⁵⁵ (SEQ ID NO: 24) and ⁷⁵⁶AANAT⁷⁶⁰ (SEQ ID NO: 25) (FIG. 9B and FIG. 9C), with the latter site providing further evidence of the relaxed sequon specificity for DmPglB. Considering this relaxed acceptor-site specificity, DmPglB's amino acid preferences at the −2 position of the sequon were investigated more systematically. This analysis took advantage of a set of plasmids encoding scFv13-R4 variants in which the −2 position of the C-terminal acceptor motif was varied to include all 20 amino acids (Ollis et al., “Engineered Oligosaccharyltransferases with Greatly Relaxed Acceptor-Site Specificity,” Nat. Chem. Biol. 10(10):816-822 (2014), which is hereby incorporated by reference in its entirety). Like DaPglB and DgPglB (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety), DmPglB exhibited greatly relaxed acceptor-site specificity in this assay (FIG. 3A and FIG. 3B). However, unlike the more variable relaxation observed for DaPglB and DgPglB previously, with certain sequons becoming strongly glycosylated and others only weakly or not at all (FIG. 10A and FIG. 10B; shown for DgPglB), DmPglB exhibited non-preferential and highly efficient glycosylation (77-100%) of all 20 sequons (FIG. 3B and FIG. 11). At this point, a catalytically inactive DmPglB variant (hereafter DmPglBmut) was also constructed by mutating two residues, D55N and E363Q, in the catalytic pocket. Sequence alignment and structural modeling indicated that these two residues corresponded to D56 and E319 in ClPglB or D54N and E316Q in CjPglB (FIG. 12), which are essential for catalytic activity (Lizak et al., “X-Ray Structure of a Bacterial Oligosaccharyltransferase,” Nature 474(7351):350-355 (2011) and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety). As expected, DmPglBmut was unable to glycosylate scFv13-R4^{DQNAT} (FIG. 3A), confirming the DmPglB-dependent nature of the glycosylation results above. To analyze acceptor-site specificity of the DmPglB enzyme in a more unbiased manner, a previously established genetic screen called glycoSNAP (glycosylation of secreted N-linked acceptor proteins) was utilized (Ollis et al., “Engineered Oligosaccharyltransferases with Greatly Relaxed Acceptor-Site Specificity,” Nat. Chem. Biol. 10(10):816-822 (2014), which is hereby incorporated by reference in its entirety). GlycoSNAP is a high-throughput colony blotting assay based on glycosylation and extracellular secretion of a reporter protein composed of E. coli YebF, a small (10 kDa in its mature form) extracellularly secreted protein (Zhang et al., “Extracellular Accumulation of Recombinant Proteins Fused to the Carrier Protein YebF in Escherichia coli,” Nat. Biotechnol. 24(1):100-104 (2006), which is hereby incorporated by reference in its entirety), or YebF fusion proteins modified with an acceptor sequon (Li et al., “Shotgun Scanning Glycomutagenesis: A Simple and Efficient Strategy Constructing and Characterizing Neoglycoproteins,” Proc. Natl. Acad. Sci. USA 118(39):e2107440118 (2021) and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety). To eliminate potentially confounding internal glycosylation in the YebF protein itself, a N24L mutant of YebF that was not glycosylated by any relaxed OST homologs was used (Ollis et al., “Substitute Sweeteners: Diverse Bacterial Oligosaccharyltransferases with Unique N-Glycosylation Site Preferences,” Sci. Rep. 5:15237 (2015) and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety).

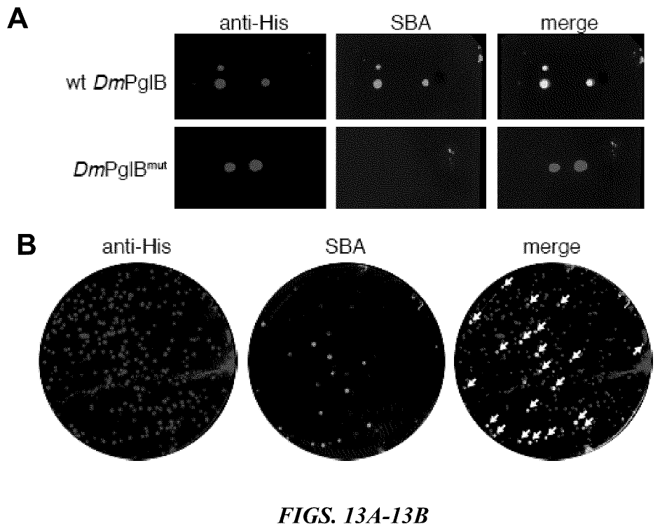

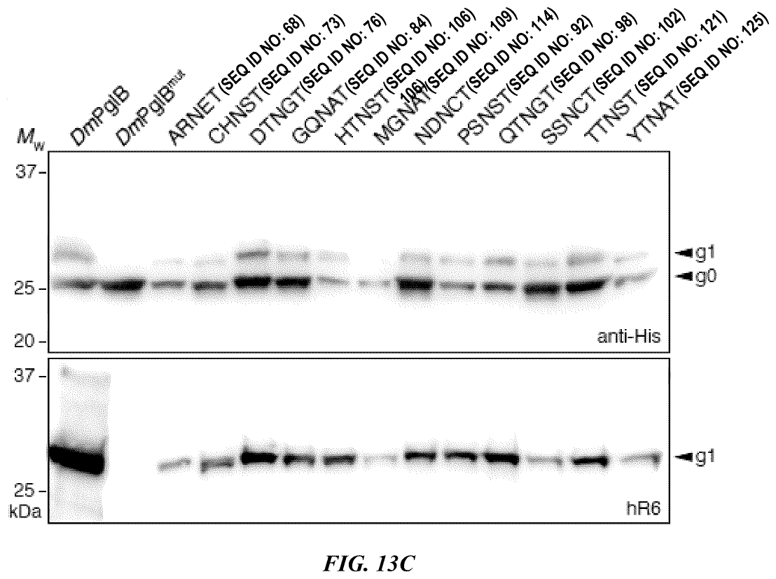

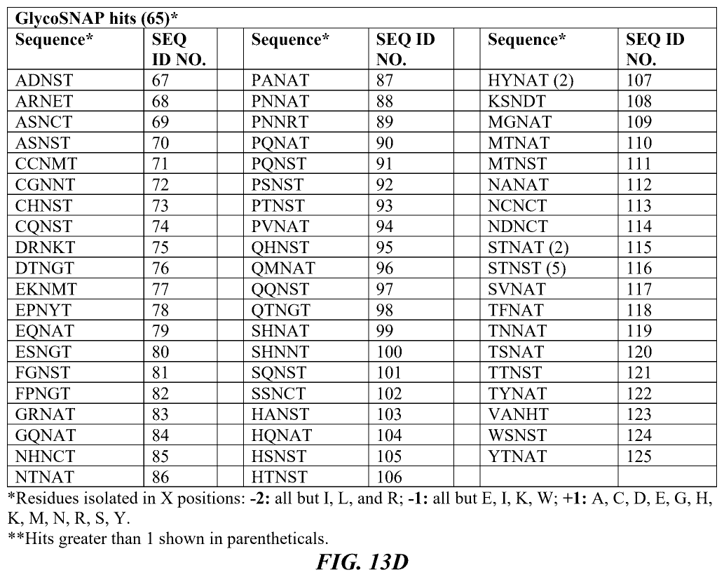

The compatibility of one such reporter fusion, YebF(N24L)-Im7 (Li et al., “Shotgun Scanning Glycomutagenesis: A Simple and Efficient Strategy Constructing and Characterizing Neoglycoproteins,” Proc. Natl. Acad. Sci. USA 118(39):e2107440118 (2021), which is hereby incorporated by reference in its entirety), with DmPglB was first evaluated in the context of a C-terminal DQNAT (SEQ ID NO: 6) sequon, with clear extracellular accumulation of glycosylated YebF(N24L)-Im7DQNAT detected for cells co-expressing wild4 type DmPglB (FIG. 13A). In contrast, there was no evidence for glycosylation of the YebF(N24L)-Im7DQNAT construct that had been secreted by cells co-expressing DmPglB^{mut}. Encouraged by this result, glycoSNAP was next used to screen a combinatorial library of acceptor-site sequences for glycosylation by DmPglB. A combinatorial library of approximately 1.1×10⁵ YebF(N24L)-Im7^{XXNXT} variants was generated by randomizing the amino acids in the −2, −1, and +1 positions of the C-terminal acceptor sequon by PCR amplification using NNK degenerate primers. The resulting library was screened by glycoSNAP replica plating to identify clones that produced glycosylated YebF(N24L)-Im7 in culture supernatants (FIG. 13B). A total of 65 positive hits were recovered (FIG. 13C and FIG. 13D) and used to generate a consensus motif representing sequons that were preferentially glycosylated by DmPglB (FIG. 3C). Overall, DmPglB exhibited highly relaxed specificity at all three variable sequon positions with only a slight preference for threonine at the −1 position and alanine or serine at the +1 position. The −2 and −1 positions showed the most variability with nearly all 20 amino acids represented at each site (FIG. 13D). Importantly, these results were in good agreement with the findings above in which DmPglB indiscriminately glycosylated every XQNAT (SEQ ID NO: 4) sequon with high efficiency.

### Example 5—Quantitative In Vitro Determination of DmPglB Catalysis

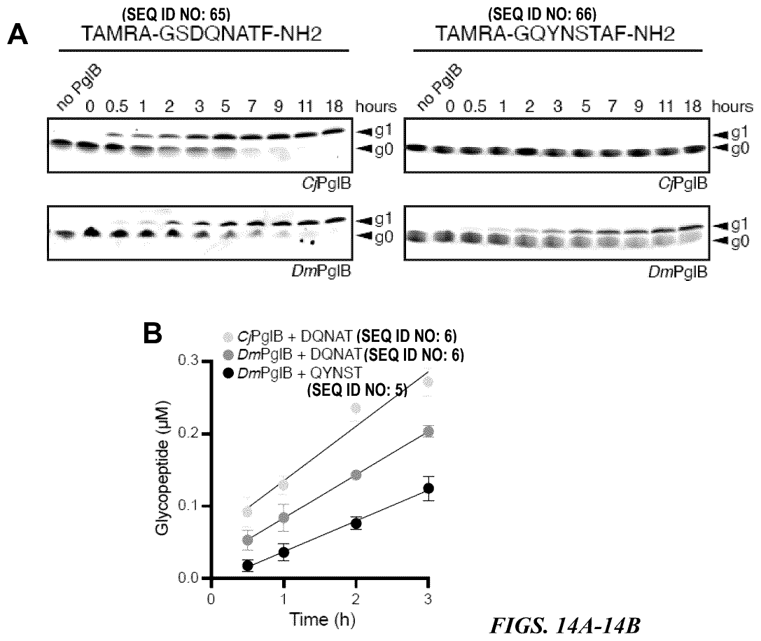

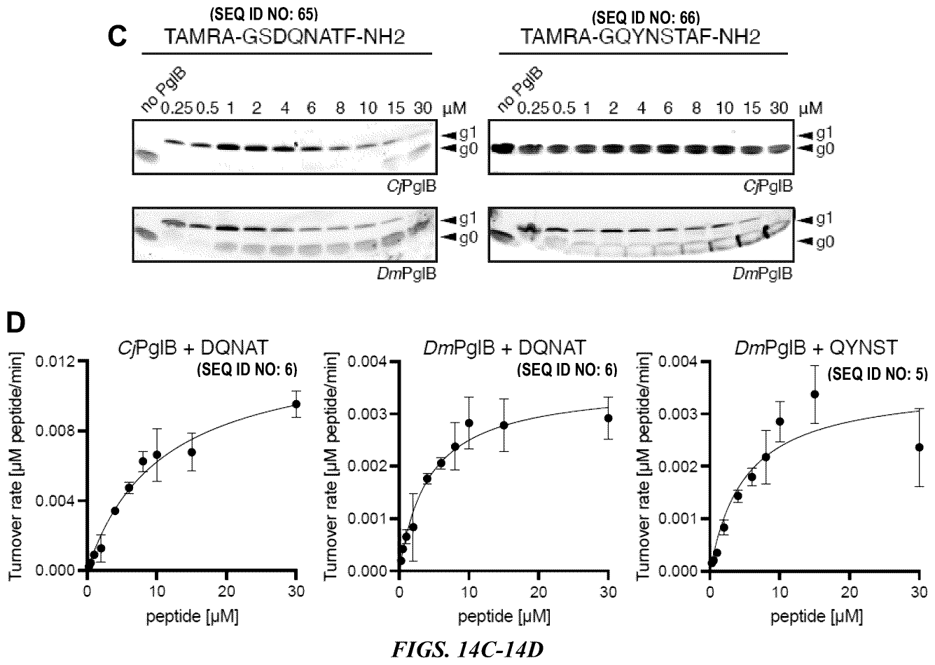

To compare the rates and Michaelis-Menten constants of DmPglB relative to the prototypic CjPglB ssOST, a fluorescently labeled peptide with either a DQNAT (SEQ ID NO: 6) or QYNST (SEQ ID NO: 5) glycosylation sequon and solvent-extracted LLOs bearing the GalNAc₅(Glc)GlcNAc glycan were employed to track the glycosylation reaction using in-gel fluorescence (Gerber et al., “Mechanism of Bacterial Oligosaccharyltransferase: In Vitro Quantification of Sequon Binding and Catalysis,” J. Biol. Chem. 288(13):8849-8861 (2013), which is hereby incorporated by reference in its entirety). The glycosylation of these peptides was determined by examining the increase of molecular weight corresponding to the addition of the approximately 1 kDa heptasaccharide using tricine-SDS-PAGE gels. Following purification of CjPglB and DmPglB, each was added to an in vitro glycosylation reaction with one of the fluorescently tagged peptide substrates along with the GalNAc₅(Glc)GlcNAc LLOs as glycan donor. The glycosylated products were separated from the unmodified substrate by gel electrophoresis, and the educt/product ratio was determined by measuring the in-gel fluorescence intensities of both educt and product bands as a function of time and peptide concentration (FIG. 14A and FIG. 14B). To determine turnover rates, time course analysis was performed, and the initial turnover rates were determined for the linear range of the reaction (FIG. 14C). Importantly, the turnover rates confirmed that the DQNAT (SEQ ID NO: 6)-containing peptide was an active substrate for both CjPglB and DmPglB, whereas the QYNST (SEQ ID NO: 5)-containing peptide was only an active substrate for DmPglB (Table 6), consistent with in vivo findings. Although kcat for DmPglB with the QYNST (SEQ ID NO: 5) sequon was approximately 30-40% lower than the turnover rates measured for each enzyme with DQNAT (SEQ ID NO: 6), it was still on par with the kcat value reported previously for CjPglB using a DANYT-containing peptide and synthetic LLO (Liu et al., “Rationally Designed Short Polyisoprenol-Linked PglB Substrates for Engineered Polypeptide and Protein N-Glycosylation,” J. Am. Chem. Soc. 136(2):566-569 (2014), which is hereby incorporated by reference in its entirety). Next, Michaelis-Menten kinetics were determined for both enzymes using increasing concentrations of peptide substrate (FIG. 14D). From this analysis, Km values of 10.7±0.98 μM were determined for CjPglB with the DQNAT (SEQ ID NO: 6) sequon and 4.30±0.95 and 5.16±1.13 for DmPglB with the DQNAT (SEQ ID NO: 6) and QYNST (SEQ ID NO: 5) sequons, respectively (Table 1). Importantly, these values were on par with Km values reported previously for CjPglB using synthetic LLO substrates (Liu et al., “Rationally Designed Short Polyisoprenol-Linked PglB Substrates for Engineered Polypeptide and Protein N-Glycosylation,” J. Am. Chem. Soc. 136(2):566-569 (2014), which is hereby incorporated by reference in its entirety) and quantifiably confirmed the relaxed acceptor site specificity of the DmPglB enzyme.

TABLE 6
Kinetic parameters for CjPglB and DmPglB
with GalNAcs(Glc)GlcNAc LLOs

| SSOT | Acceptor sequon | K_{cat} (h⁻¹) | K_{M} (μM) |
|---|---|---|---|
| CjPglB | DQNAT | 0.42 ± 0.08 | 10.7 ± 0.98 |
| | (SEQ ID NO: 6) | | |
| DmPglB | DQNAT | 0.33 ± 0.05 | 4.3 ± 0.95 |
| | (SEQ ID NO: 6) | | |
| CjPGlB | QYNST | n.a. | n.a. |
| | (SEQ ID NO: 5) | | |
| DmPglB | QYNST | 0.24 ± 0.02 | 5.16 ± 1.13 |
| | (SEQ ID NO: 5) | | |

^{a}K_{M} and k_{cat} values calculated from in vitro glycosylation data shown in FIGS. 14A-14D.
Reactions were performed using extracted GalNAc₅(Glc)GlcNAc LLOs and fluorescent TAMRA-GSDQNATF-NH₂ (SEQ ID NO: 65) or TAMRA-GQYNSTAF-NH₂ (SEQ ID NO: 66) as substrates.
Data are the average of technical replicates (n = 3) ± SD.
In the case of CjPglB with QYNST (SEQ ID NO: 5) sequon, no activity (n.a.) was detected.

### Example 6—DmPglB Structure Contains Both Bacterial and Eukaryotic Features

To better understand the observed functional differences for DmPglB relative to other OSTs, a structural model of DmPglB was generated using the AlphaFold2 protein structure prediction algorithm implemented with ColabFold (Mirdita et al., “ColabFold: Making Protein Folding Accessible to All,” Nat. Methods 19(6):679-682 (2022) and Jumper et al., “Highly Accurate Protein Structure Prediction with AlphaFold,” Nature 596(7873):583-589 (2021), which is hereby incorporated by reference in its entirety). Comparing the predicted structure of DmPglB with the solved structure of ClPglB (Lizak et al., “X-Ray Structure of a Bacterial Oligosaccharyltransferase,” Nature 474(7351):350-355 (2011), which is hereby incorporated by reference in its entirety) revealed clear variations in the structures of the catalytic pockets.

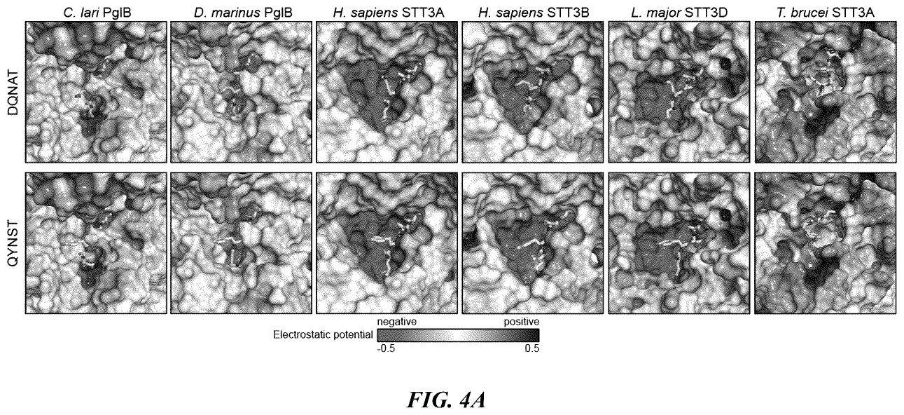

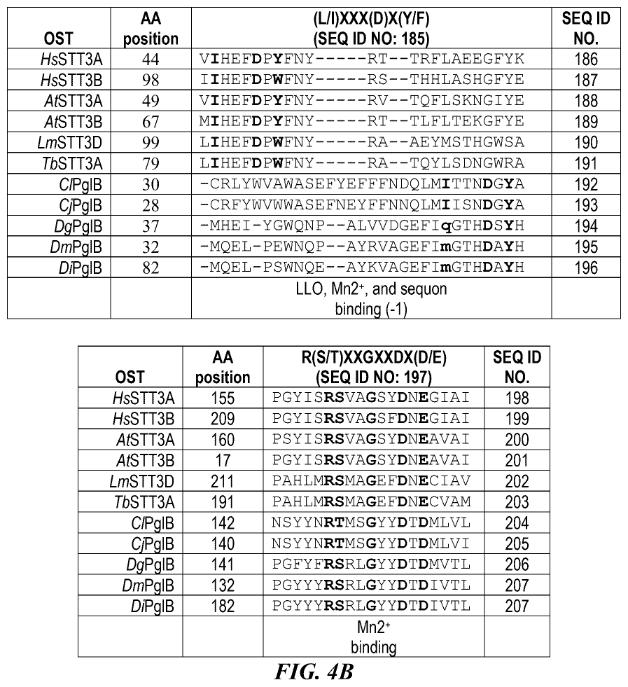

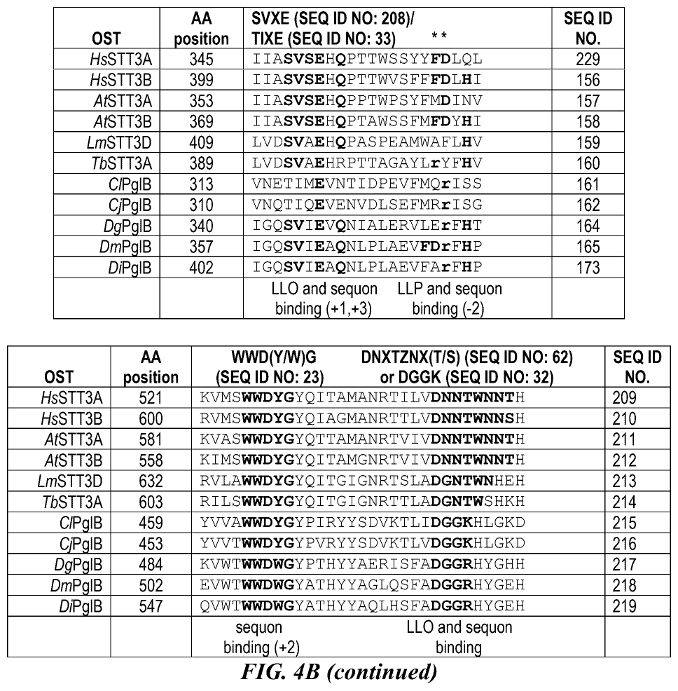

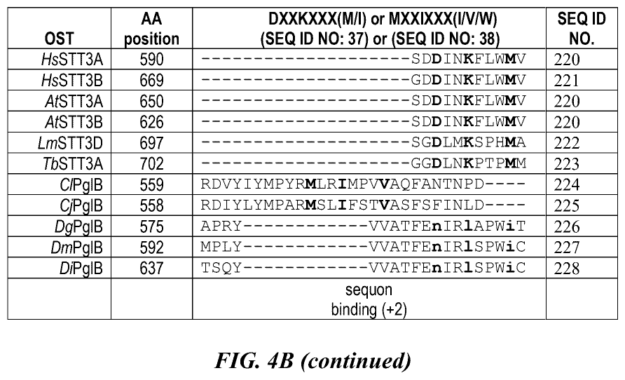

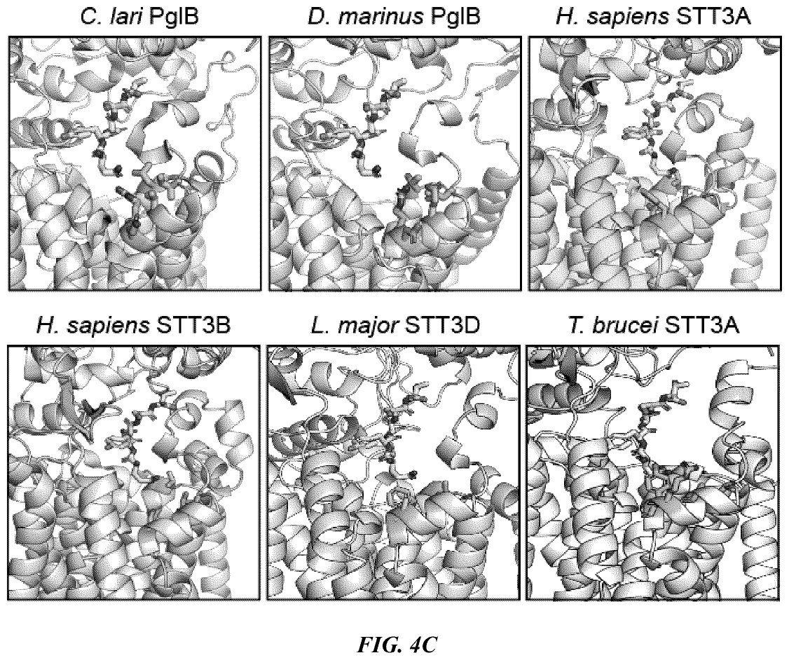

Based on electrostatic surface calculations (Baker et al., “Electrostatics of Nanosystems: Application to Microtubules and the Ribosome,” Proc. Natl. Acad. Sci. USA 98:10037-10041 (2001), which is hereby incorporated by reference in its entirety), it is apparent that the entrance to the peptide-binding cavity that hosts the −2 position of the acceptor sequon is positively charged in ClPglB but neutral in DmPglB (FIG. 4A). This difference in surface charge results from residues in the vicinity of the arginine at position 331 in ClPglB (R375 in DmPglB), which is strongly conserved in bacterial ssOSTs (FIG. 4B) and provides a salt bridge to the aspartic acid in a bound DQNATF (SEQ ID NO: 64) substrate peptide in the ClPglB crystal structure (Lizak et al., “X-Ray Structure of a Bacterial Oligosaccharyltransferase,” Nature 474(7351):350-355 (2011), which is hereby incorporated by reference in its entirety). Specifically, in the case of ClPglB, R331 is surrounded by primarily hydrophobic residues (1323, V327, and L374) that cluster to form a positively charged patch in this region of the protein (FIG. 4A and FIG. 4C). Conversely, the same region in DmPglB is significantly more neutral due to the occurrence of negatively charged and neutral amino acids (L367, E371, D374 and T418) that surround R375, providing a possible explanation for the more relaxed substrate specificity of this enzyme. Another visible difference is the peptide-binding cavity in DmPglB, which is more spacious and lined with more negatively charged residues than the cavity in ClPglB. It is worth noting that structural models of eukaryotic STT3s, which themselves do not require an acidic residue in the −2 position of the sequon, exhibited features akin to DmPglB including an even more voluminous peptide-binding cavity with a similarly neutral entrance and a highly negatively charged lining (FIG. 4A).

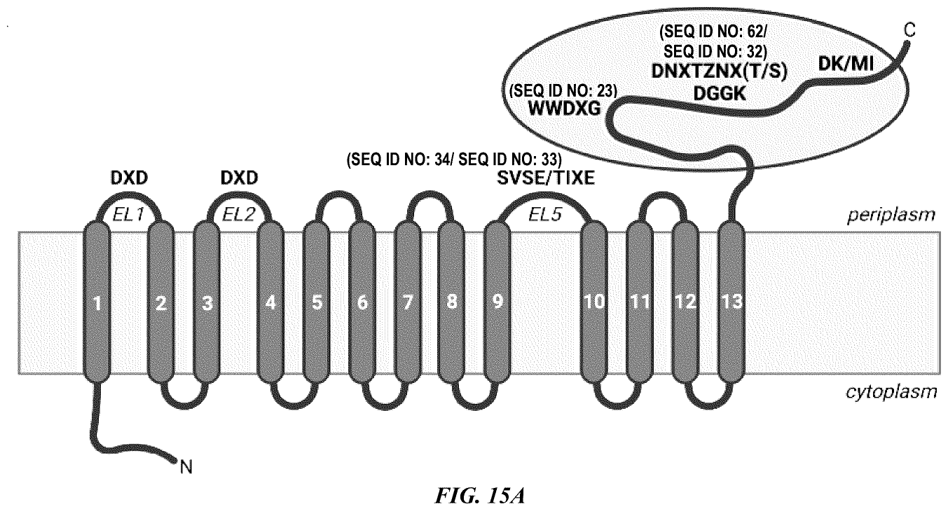

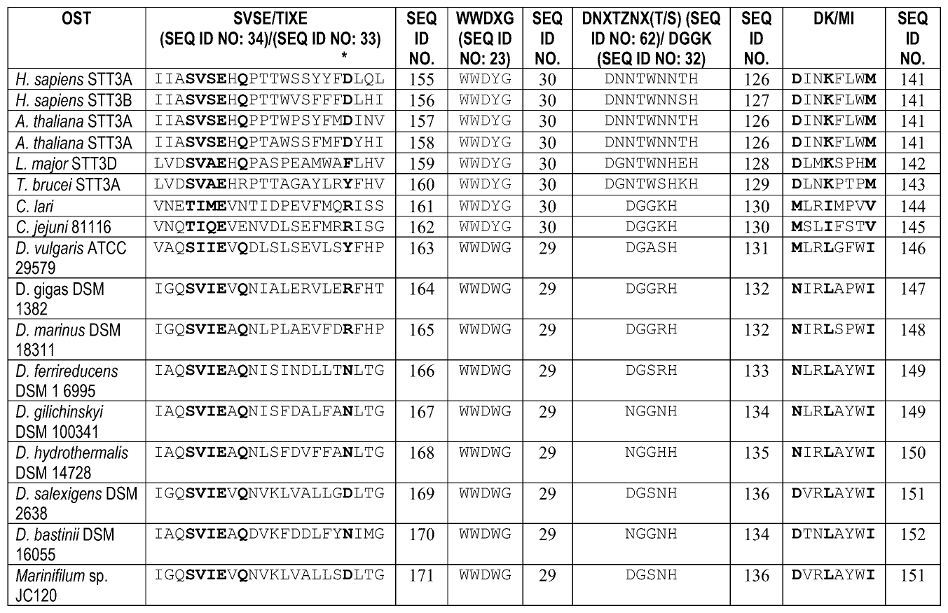

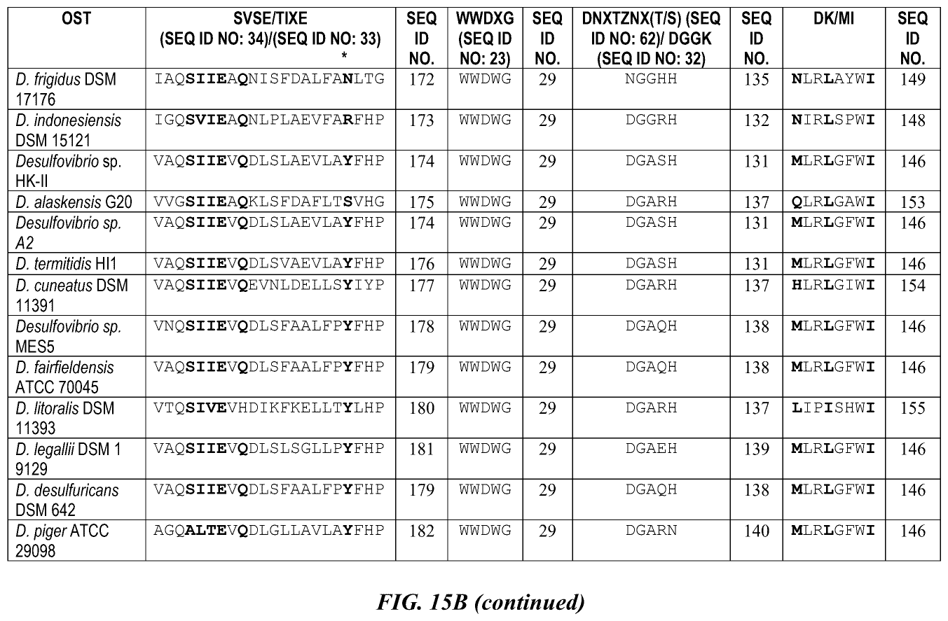

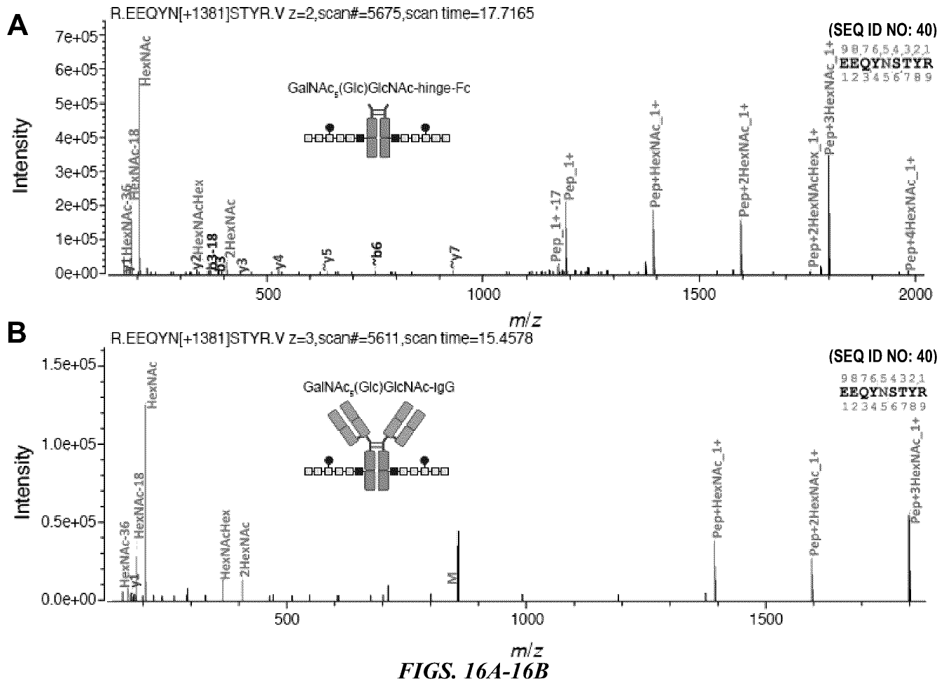

Multiple sequence alignment revealed that the Desulfovibrio PglBs possessed all the short, conserved motifs that have been previously documented for OSTs across all kingdoms, albeit with subtle deviations from the Campylobacter and eukaryotic OSTs including WWDWG (SEQ ID NO: 29) instead of WWDYG (SEQ ID NO: 30), DGGR (SEQ ID NO: 31) instead of DGGK (SEQ ID NO: 32), and NL instead of DK/MI (FIG. 4B, FIG. 15A, and FIG. 15B). A more dramatic difference was observed for the SVSE (SEQ ID NO: 34)/TIXE (SEQ ID NO: 33) motif, which occurs in the fifth external loop (EL5) and is involved in recognizing sequons at the main-chain level with the glutamic acid serving as a coordination switch that responds to ligand binding (Taguchi et al., “The Structure of an Archaeal Oligosaccharyltransferase Provides Insight into the Strict Exclusion of Proline from the N-Glycosylation Sequon,” Commun. Biol. 4(1):941 (2021), which is hereby incorporated by reference in its entirety). It has been widely reported that the conserved SVSE (SEQ ID NO: 34) motif is unique to eukaryotes whereas the conserved TIXE (SEQ ID NO: 33) motif is confined to archaeal and cubacterial OSTs. Surprisingly, all Desulfovibrio PglBs including DgPglB, DmPglB, and DiPglB possessed SVIE (SEQ ID NO: 35)/SIIE (SEQ ID NO: 36) motifs that were more like the eukaryotic SVSE (SEQ ID NO: 34) motif than the canonical bacterial TIXE (SEQ ID NO: 33) motifs found in ClPglB and CjPglB (FIG. 4B, FIG. 15A, and FIG. 15B). Moreover, in eukaryotic and Desulfovibrio OSTs a highly conserved glutamine was observed located two residues downstream of this motif, with the Desulfovibrio PglB homologs also possessing a highly conserved glutamine immediately upstream of the motif.

### Example 7—Glycosylation of Native QYNST Sequon in Human Fc Domains

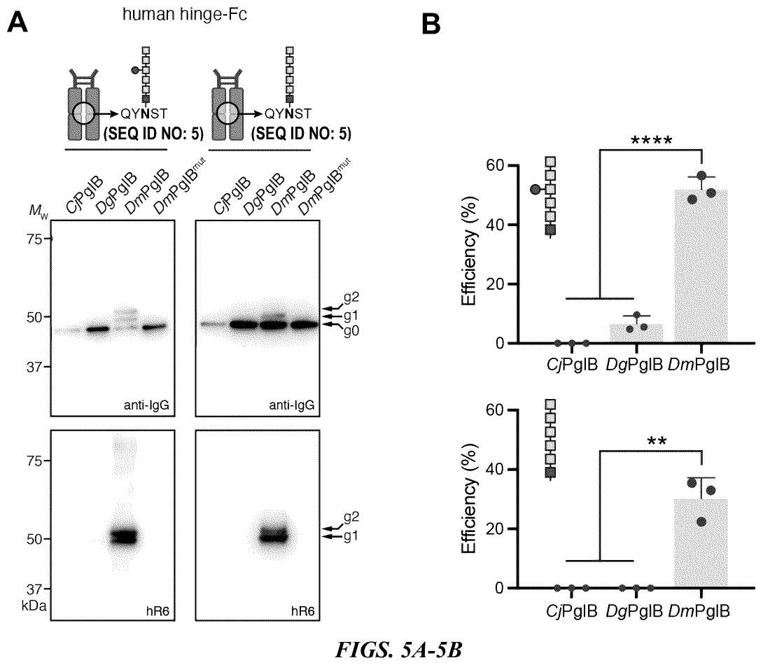

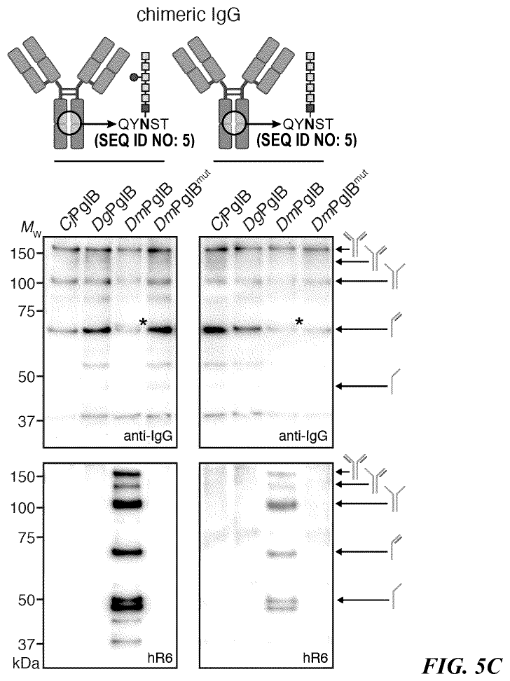

Encouraged by the ability of DmPglB to recognize minimal N−X−T motifs, the extent to which DmPglB could glycosylate the native QYNST (SEQ ID NO: 5) site found in the Fc region of an IgG antibody was next investigated. To this end, a pTrc99S-based plasmid that encoded the native Fc region and hinge derived from human IgG1 (hereafter hinge-Fc) was generated. For the N-glycan, the same pMW07-pglΔBCDEF plasmid from above as well as a derivative, plasmid pMW07-pglΔBICDEF, that produces GalNAc₅GlcNAc without the branching glucose were utilized. This latter glycan was added because it facilitates enzymatic removal of GalNAc₅ to reveal a GlcNAc “primer” that can be used for chemoenzymatic glycan remodeling (Schwarz et al., “A Combined Method for Producing Homogeneous Glycoproteins with Eukaryotic N-Glycosylation,” Nat. Chem. Biol. 6(4):264-266 (2010), which is hereby incorporated by reference in its entirety). For the PglB homologs, DmPglB was evaluated alongside both CjPglB and DgPglB, with the latter two enzymes having been shown previously to glycosylate Fc domains but with very low efficiency (Ollis et al., “Substitute Sweeteners: Diverse Bacterial Oligosaccharyltransferases with Unique N-Glycosylation Site Preferences,” Sci. Rep. 5:15237 (2015); Fisher et al., “Production of Secretory and Extracellular N-Linked Glycoproteins in Escherichia Coli,” Appl. Environ. Microbiol. 77(3):871-881 (2011); Schwarz et al., “A Combined Method for Producing Homogeneous Glycoproteins with Eukaryotic N-Glycosylation,” Nat. Chem. Biol. 6(4):264-266 (2010); Schwarz et al., “Relaxed Acceptor Site Specificity of Bacterial Oligosaccharyltransferase In Vivo,” Glycobiology 21(1):45-54 (2011); and Valderrama-Rincon et al., “An Engineered Eukaryotic Protein Glycosylation Pathway in Escherichia Coli,” Nat. Chem. Biol. 8(5):434-436 (2012), which are hereby incorporated by reference in their entirety). Each of the PglB homologs were expressed from pMLBAD as above. In agreement with previous work (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety), CjPglB was unable to glycosylate the native QYNST (SEQ ID NO: 5) sequon in the hinge-Fc with either of the tested N-glycan structures as revealed by non-reducing immunoblot analysis using an anti-IgG antibody and hR6 serum for detection (FIG. 5A). In the case of DgPglB, there was also no glycosylation detected with the GalNAc5GlcNAc glycan and only weak glycosylation (<7%) with the GalNAc₅(Glc)GlcNAc glycan (FIG. 5A and FIG. 5B), consistent with earlier observations in which DgPglB only glycosylated a small fraction (<5%) of hinge-Fc molecules (Ollis et al., “Substitute Sweeteners: Diverse Bacterial Oligosaccharyltransferases with Unique N-Glycosylation Site Preferences,” Sci. Rep. 5:15237 (2015), which is hereby incorporated by reference in its entirety). In stark contrast, DmPglB glycosylated the hinge-Fc regardless of the N-glycan used, in agreement with the extremely relaxed acceptor-site specificity observed above for this ssOST. Densitometric analysis revealed that DmPglB glycosylated the hinge-Fc with an efficiency of 52% when using GalNAc₅(Glc)GlcNAc and 30% with GalNAc₅GlcNAc, which reflected a statistically significant increase over the very low glycosylation efficiency achieved with DmPglB (FIG. 5B).

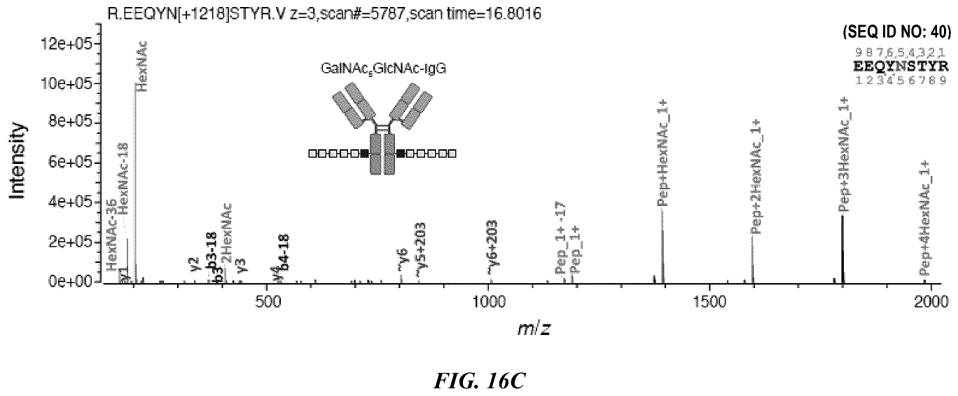

Importantly, this activity was completely absent in cells carrying the DmPglBmut variant, confirming the OST-dependent nature of the glycosylation. Moreover, the observation of doubly and singly glycosylated hinge-Fc indicated that a mixture of fully and hemi-glycosylated products, respectively, were generated under the conditions tested, with roughly equal quantities of both based on the comparable g2 and g1 band intensities in the anti-glycan blot. To unequivocally prove glycosylation of the native QYNST (SEQ ID NO: 5) sequon in hinge-Fc by DmPglB, LC-MS/MS analysis of the glycosylation products was performed under reduced and protease-digested conditions. The MS/MS spectrum of a tryptic peptide (⁹⁹EEQYNSTYR¹⁰⁷ (SEQ ID NO: 40)) containing the known glycosylation sequon conclusively revealed the presence of a HexNAc6Hex1 structure, consistent with the GalNAc₅(Glc)GlcNAc glycan (FIG. 16A).

Whether DmPglB could glycosylate a full-length IgG1 antibody, namely YMF10, which is a chimeric IgG clone (murine VH and VL regions and human constant regions) with high affinity and specificity for Bacillus anthracis protective antigen (PA) (Mazor et al., “Isolation of Engineered, Full-Length Antibodies from Libraries Expressed in Escherichia coli,” Nat. Biotechnol. 25(5):563-565 (2007), which is hereby incorporated by reference in its entirety) was next investigated. YMF10 was chosen because it can be expressed in the E. coli periplasm at high levels, and its heavy chain (HC) and light chain (LC) can be properly assembled into a functional full-length IgG. To ensure efficient IgG expression, JUDE-1 E. coli cells carrying plasmid pMAZ360-YMF10-IgG were used as described previously (Mazor et al., “Isolation of Engineered, Full-Length Antibodies from Libraries Expressed in Escherichia coli,” Nat. Biotechnol. 25(5):563-565 (2007), which is hereby incorporated by reference in its entirety). These cells were further transformed with plasmid pMLBAD encoding a PglB homolog and either pMW07-pglΔBCDEF or pMW07-pglΔBICDEF encoding the N-glycan biosynthesis genes. Non-reducing immunoblot analysis revealed formation of fully assembled heterotetrameric YMF10 as well as other intermediate products for each of the strain/plasmid combinations tested (FIG. 5B), in line with expression patterns observed previously (Mazor et al., “Isolation of Engineered, Full-Length Antibodies from Libraries Expressed in Escherichia coli,” Nat. Biotechnol. 25(5):563-565 (2007). Importantly, only cells carrying DmPglB were capable of YMF10 glycosylation as evidenced by detection of HC-linked glycans with hR6 serum, whereas no glycosylation was observed for cells carrying cither CjPglB or DgPglB (FIG. 5B). Although all products containing at least one HC were detected by hR6 serum, the fully assembled IgG tetramer was one of the major glycoforms along with the HC-HC and HC LC dimers based on relative band intensities. While it was difficult to see a band shift in the anti-IgG blot indicative of glycosylation of the full-length protein due to poor resolution at higher molecular weights (>100 kDa), a band shift was observed for the half antibody product (HC-LC dimer) at approximately 70 kDa. As expected, there was no detectable glycosylation activity when the catalytically inactive mutant DmPglBmut was substituted for wt DmPglB. Further confirmation of IgG glycosylation was obtained by LC-MS/MS analysis of reduced and digested IgG-containing samples. Specifically, the MS/MS spectrum confirmed glycosylation of a tryptic peptide (²⁹³EEQYNSTYR³⁰¹ (SEQ ID NO: 40)) containing the known glycosylation sequon and modified with HexNAc6Hex1 or HexNAc6, consistent with the GalNAc₅(Glc)GlcNAc and GalNAc₅GlcNAc glycans, respectively (FIG. 16B and FIG. 16C). From the LC-MS/MS analysis, the glycosylation efficiency of YMF10 was estimated to range from 10-14% with these two N-glycans.

### Example 8—Remodeling Bacteria-Derived IgG1-Fc with Eukaryotic N-Glycans

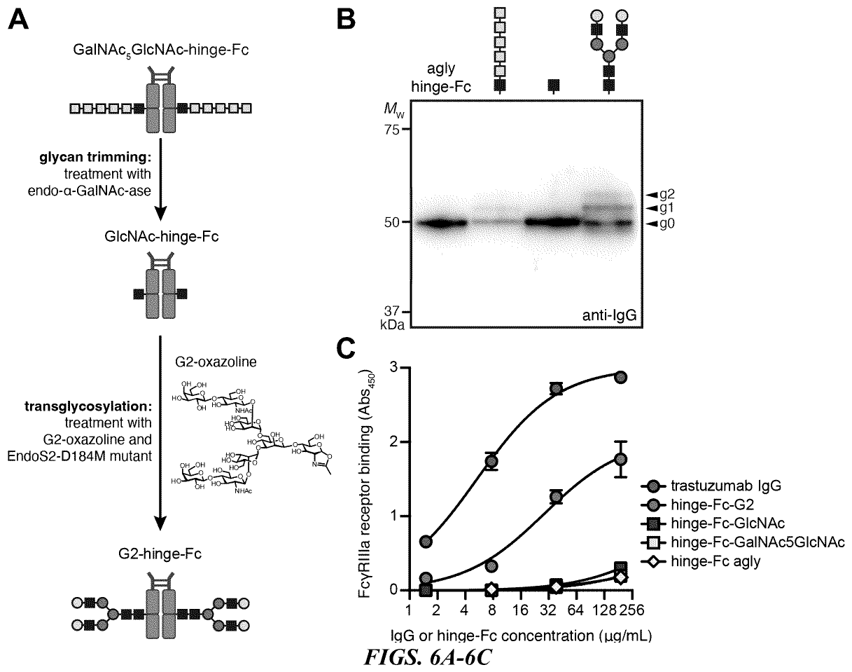

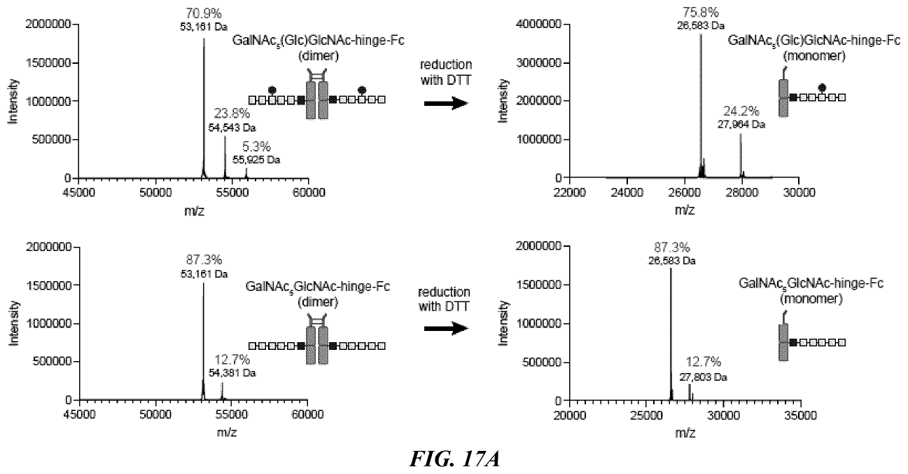

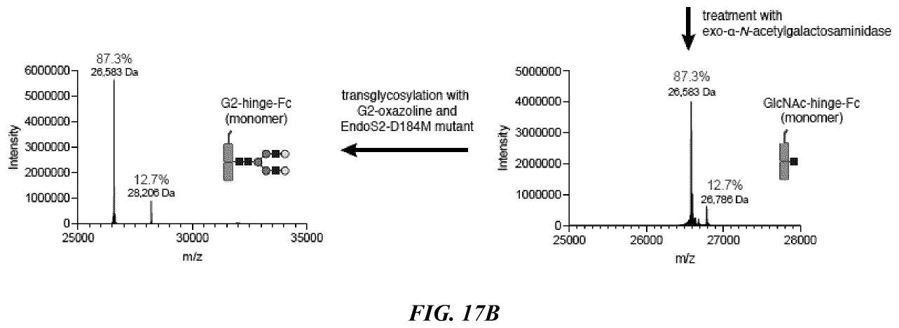

Upon confirming the ability of DmPglB to glycosylate the authentic QYNST (SEQ ID NO: 5) sequon in human hinge-Fc, whether the installed GalNAc5GlcNAc glycan could be transformed into a more biomedically relevant glycoform was next investigated (FIG. 6A). To this end, a previously described glycan remodeling strategy that used engineered E. coli to produce glycoproteins bearing GalNAc₅GlcNAc glycans, which were subsequently trimmed and chemoenzymatically remodeled in vitro by an enzymatic transglycosylation reaction, was adopted. Using this method, it was possible to install eukaryotic N-glycans including an asialo afucosylated complex-type biantennary glycan (Gal₂GlcNAc₂Man₃GlcNAc₂; G2) onto a model bacterial acceptor protein (Schwarz et al., “A Combined Method for Producing Homogeneous Glycoproteins with Eukaryotic N-Glycosylation,” Nat. Chem. Biol. 6(4):264-266 (2010), which is hereby incorporated by reference in its entirety. However, this method could not be extended to a human CH2 domain because of the low glycosylation efficiency (<5%) achieved with CjPglB, even after the introduction of a preferred DFNST sequon in place of QYNST (SEQ ID NO: 5) in this protein. Here, it was hypothesized that transglycosylation by this strategy would be possible with the glycosylated hinge-Fc protein according to the present disclosure due to the much higher glycosylation efficiency (approximately 30-50%) achieved with DmPglB. To test this hypothesis, the protein A-purified hinge-Fc bearing GalNAc₅GlcNAc was first subjected to enzymatic trimming with exo-α-N-acetylgalactosaminidase, with GalNAc removal being continuously monitored by LC-ESI-MS (FIG. 17A) and confirmed by immunoblot analysis (FIG. 6B). The resulting hinge-Fc bearing only a GlcNAc stump was then subjected to transglycosylation catalyzed by the glycosynthase mutant, EndoS2-D184M (Li et al., “Glycosynthase Mutants of Endoglycosidase S2 Show Potent Transglycosylation Activity and Remarkably Relaxed Substrate Specificity for Antibody Glycosylation Remodeling,” J. Biol. Chem. 291(32):16508-16518 (2016), which is hereby incorporated by reference in its entirety), with preassembled G2-oxazoline as donor substrate (Schwarz et al., “A Combined Method for Producing Homogeneous Glycoproteins with Eukaryotic N-Glycosylation,” Nat. Chem. Biol. 6(4):264-266 (2010), which is hereby incorporated by reference in its entirety) in a reaction that was again monitored by LC-ESI-MS (FIG. 17B) and confirmed by immunoblot analysis (FIG. 6B). This sequence of steps produced a hinge-Fc protein bearing the G2 glycoform (G2-hinge-Fc).

To evaluate the functional consequences of installing eukaryotic glycans onto the E. coli-derived hinge-Fc, the binding affinity between different hinge-Fc glycoforms and a human Fc gamma receptor (FcγR) was investigated. Specifically, the clinically relevant FcγRIIIa-V158 allotype (Ravetch and Perussia, “Alternative Membrane forms of Fc Gamma RIII(CD16) on Human Natural Killer Cells and Neutrophils. Cell Type-Specific Expression of two Genes that Differ in Single Nucleotide Substitutions,” J. Exp. Med. 170(2):481-497 (1989), which is hereby incorporated by reference in its entirety) was tested because it is the high-affinity allele and interactions between this receptor and different IgG subclasses have been extensively studied (Bruhns et al., “Specificity and Affinity of Human Fcgamma Receptors and their Polymorphic Variants for Human IgG Subclasses,” Blood 113(16):3716-3725 (2009) and de Taeye et al., “FcγR Binding and ADCC Activity of Human IgG Allotypes,” Front. Immunol. 6:11:740 (2020), which are hereby incorporated by reference in their entirety). It is also worth noting that glycosylated hinge-Fc antibodies including those containing terminal galactose residues, such as G2, exhibit affinity for FcγRIIIa (Wei et al., “Glycoengineering of Human IgG1-Fc through Combined Yeast Expression and In Vitro Chemoenzymatic Glycosylation,” Biochemistry 47(39):10294-10304 (2008), which is hereby incorporated by reference in its entirety). In total, four E. coli-derived glycoprotein forms were investiated: aglycosylated hinge-Fc, glycosylated GalNAc5GlcNAc-hinge-Fc, GlcNAc-hinge-Fc, and G2-hinge-Fc. Among these glycoforms, G2-hinge-Fc displayed the highest binding affinity for FcγRIIIA-V158 as determined by enzyme-linked immunosorbent assay (ELISA), with a half-maximal effective concentration (EC₅₀) of 28.5±3.2 nM (FIG. 6C). In contrast, little to no binding above background was observed for the trimmed GlcNAc-hinge-Fc, untrimmed hinge-Fc bearing the GalNAc5GlcNAc glycan, and aglycosylated hinge-Fc, confirming the importance of the human N-glycan structure on FcγRIIIA-V158 binding. By way of comparison, an EC₅₀ of 5.4±0.6 nM was measured for commercial trastuzumab (FIG. 6C), consistent with the EC₅₀ value of 1.4 nM for FcγRIIIA-V158 that was measured for another IgG product, rituximab, following glycan remodeling to acquire the G2 glycan (Li et al., “Modulating IgG Effector Function by Fc Glycan Engineering,” Proc. Natl. Acad. Sci. USA 114(13):3485-3490 (2017), which is hereby incorporated by reference in its entirety). The weaker FcγRIIIA affinity of the G2-hinge-Fc relative to these full-length IgGs may be due to differences in their glycosylation levels and/or the absence of Fab domains in hinge-Fc that stabilize IgG-FcγRIIIA interactions (Kurogochi et al., “Glycoengineered Monoclonal Antibodies with Homogeneous Glycan (M3, G0, G2, and A2) Using a Chemoenzymatic Approach have Different Affinities for FcγRIIIa and Variable Antibody-Dependent Cellular Cytotoxicity Activities,” PLos One 10(7):e0132848 (2015), which is hereby incorporated by reference in its entirety). Regardless, these results provide proof-of concept for chemoenzymatic conversion of E. coli-derived IgG-Fc glycans into glycoforms that preserve important Fc effector functions.

### Discussion of Examples 1-8

The engineered expression of glycosylated antibodies in E. coli depends on OSTs that can install N-linked glycans within the QYNST (SEQ ID NO: 5) sequon of the IgG C_{H}2 domain. To this end, a previously uncharacterized ssOST, DmPglB, that was able to glycosylate minimal N−X−S/T sequons with high efficiency and without preference for the residues in the −2, −1 or +1 positions, was identified. In fact, the breadth of sequons recognized by DmPglB and the efficiency with which they were modified was unmatched by any of the approximately 50 bacterial ssOSTs that have been tested here and elsewhere (Kowarik et al., “Definition of the Bacterial N-Glycosylation Site Consensus Sequence,” EMBO J. 25(9):1957-1966 (2006); Ollis et al., “Substitute Sweeteners: Diverse Bacterial Oligosaccharyltransferases with Unique N-Glycosylation Site Preferences,” Sci. Rep. 5:15237 (2015); Schwarz et al., “Relaxed Acceptor Site Specificity of Bacterial Oligosaccharyltransferase In Vivo,” Glycobiology 21(1):45-54 (2011); Ielmini and Feldman, “Desulfovibrio desulfuricans PglB Homolog Possesses Oligosaccharyltransferase Activity with Relaxed Glycan Specificity and Distinct Protein Acceptor Sequence Requirements,” Glycobiology 21(6):734-742 (2011); and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety.

Importantly, DmPglB promoted glycosylation of the native QYNST (SEQ ID NO: 5) motif in a human hinge-Fc fragment and a full-length, chimeric IgG antibody, with efficiencies that ranged from approximately 30-52% and approximately 10-14%, respectively, which were significantly higher than any of the efficiencies reported previously for PglB-mediated Fc glycosylation in E. coli (Ollis et al., “Substitute Sweeteners: Diverse Bacterial Oligosaccharyltransferases with Unique N-Glycosylation Site Preferences,” Sci. Rep. 5:15237 (2015); Fisher et al., “Production of Secretory and Extracellular N-Linked Glycoproteins in Escherichia Coli,” Appl. Environ. Microbiol. 77(3):871-881 (2011); Schwarz et al., “A Combined Method for Producing Homogeneous Glycoproteins with Eukaryotic N-Glycosylation,” Nat. Chem. Biol. 6(4):264-266 (2010); Schwarz et al., “Relaxed Acceptor Site Specificity of Bacterial Oligosaccharyltransferase In Vivo,” Glycobiology 21(1):45-54 (2011); and Valderrama-Rincon et al., “An Engineered Eukaryotic Protein Glycosylation Pathway in Escherichia Coli,” Nat. Chem. Biol. 8(5):434-436 (2012), which are hereby incorporated by reference in their entirety). Although the installed glycans were bacterial-type structures, this limitation was sidestepped by in vitro chemoenzymatic transformation of bacterial GalNAc₅GlcNAc into complex-type G2, a glycan that is known to enhance ADCC activity in vitro and anticancer efficacy in vivo (Niwa et al., “Defucosylated Chimeric Anti-CC Chemokine Receptor 4 IgG1 with Enhanced Antibody-Dependent Cellular Cytotoxicity Shows Potent Therapeutic Activity to T-cell Leukemia and Lymphoma,” Cancer Res. 64:2127-2133 (2004), which is hereby incorporated by reference in its entirety). The complete conversion to G2 on hinge-Fc observed here was significantly more efficient than the roughly 50% conversion achieved with a model bacterial glycoprotein (Schwarz et al., “A Combined Method for Producing Homogeneous Glycoproteins with Eukaryotic N-Glycosylation,” Nat. Chem. Biol. 6(4):264-266 (2010), which is hereby incorporated by reference in its entirety). This difference was presumably due to the use of a more efficient glycosynthase mutant, EndoS2-D184M, that potently remodels antibodies with complex-type glycans including G2 (Li et al., “Glycosynthase Mutants of Endoglycosidase S2 Show Potent Transglycosylation Activity and Remarkably Relaxed Substrate Specificity for Antibody Glycosylation Remodeling,” J. Biol. Chem. 291(32):16508-16518 (2016), which is hereby incorporated by reference in its entirety). Importantly, the remodeled G2-hinge-Fc engaged FcγRIIIa while the hinge-Fc bearing the bacterial glycan did not, demonstrating the potential of this strategy for creating antibodies with native effector functions.

While the precise sequence determinants responsible for the unique substrate specificity of DmPglB remain to be experimentally determined, it was hypothesized that acceptor substrate selection is governed in part by the EL5 loop including the SVSE (SEQ ID NO: 34)/TIXE (SEQ ID NO: 33) motif and neighboring residues. This hypothesis is supported by structural models that showed the SVSE (SEQ ID NO: 34)/TIXE (SEQ ID NO: 33) motifs of bacterial and eukaryotic OSTs in close proximity to the acceptor peptide. This positioning is consistent with recently determined crystal structures of archaeal and bacterial ssOSTs, namely AglB from Archacoglobus fulgidus (AfAglB) and ClPglB, respectively, with bound substrate peptide, which revealed that the TIXE (SEQ ID NO: 33) motif lies side-by-side in an anti-parallel β-sheet configuration with the sequon and forms two interchain hydrogen bonds with the +1 and +3 residues of the sequon (Taguchi et al., “The Structure of an Archaeal Oligosaccharyltransferase Provides Insight into the Strict Exclusion of Proline from the N-Glycosylation Sequon,” Commun. Biol. 4(1):941 (2021) and Napiorkowska et al., “Molecular Basis of Lipid-Linked Oligosaccharide Recognition and Processing by Bacterial Oligosaccharyltransferase,” Nat. Struct. Mol. Biol. 24(12):1100-1106 (2017), which are hereby incorporated by reference in their entirety). Interestingly, whereas ClPglB and CjPglB each possess a canonical bacterial TIXE (SEQ ID NO: 33) motif and follow the minus two rule, the DgPglB, DiPglB, and DmPglB enzymes possess eukaryotic-like SVIE (SEQ ID NO: 35) motifs. This motif in Desulfovibrio ssOSTs may contribute to their more eukaryotic-like sequon requirements relative to Campylobacter ssOSTs. However, the fact that archaeal OSTs also possess a TIXE (SEQ ID NO: 33) motif and yet do not require an acidic residue in the −2 position of the sequon indicates that this motif alone is insufficient to explain the differences in sequon preference among these OSTs. Additional residues in the vicinity of the SVSE (SEQ ID NO: 34)/TIXE (SEQ ID NO: 33) motif might also be important in determining acceptor substrate preferences. In support of this notion, alanine scanning mutagenesis of the EL5 loop of AfAglB confirmed that the TIXE (SEQ ID NO: 33) motif as well five adjacent downstream residues that are positioned near the −2 position of the acceptor peptide are essential for glycosylation activity (Taguchi et al., “The Structure of an Archaeal Oligosaccharyltransferase Provides Insight into the Strict Exclusion of Proline from the N-Glycosylation Sequon,” Commun. Biol. 4(1):941 (2021), which is hereby incorporated by reference in its entirety). These residues are in the immediate vicinity of the highly conserved arginine that, in ClPglB, forms a stabilizing salt bridge with the aspartic acid in the −2 position of the sequon (Lizak et al., “X-Ray Structure of a Bacterial Oligosaccharyltransferase,” Nature 474(7351):350-355 (2011), which is hereby incorporated by reference in its entirety). This residue appears to be a key regulator of sequon selection based on mutagenesis studies in which substitution of the analogous arginine in CjPglB or DgPglB with residues such as leucine or asparagine was sufficient to reprogram the −2 preferences of each enzyme (Ollis et al., “Substitute Sweeteners: Diverse Bacterial Oligosaccharyltransferases with Unique N-Glycosylation Site Preferences,” Sci. Rep. 5:15237 (2015) and Ollis et al., “Engineered Oligosaccharyltransferases with Greatly Relaxed Acceptor-Site Specificity,” Nat. Chem. Biol. 10(10):816-822 (2014), which are hereby incorporated by reference in their entirety. Another key feature in sequon selection may be the electrostatic charge of this region of the enzyme, which forms the peptide-binding cavity and is more neutral in DmPglB and eukaryotic OSTs but positively charged in ClPglB. A more spacious peptide-binding cavity in DmPglB may also contribute to its ability to accommodate sequons having bulkier sidechains such as the aromatic residue at −1 of QYNST (SEQ ID NO: 5).

It has long been known that the E. coli periplasm can support the proper assembly of antibody HC and LC (Simmons et al., “Expression of Full-Length Immunoglobulins in Escherichia coli: Rapid and Efficient Production of Aglycosylated Antibodies,” J. Immunol. Methods 263 (1-2):133-147 (2002), which is hereby incorporated by reference in its entirety). However, while E. coli-derived antibodies bind strongly to their cognate antigens and the neonatal Fc receptor (FcRn), they show no significant binding to complement component 1q (C1q) or FcγRs due to lack of glycosylation (Simmons et al., “Expression of Full-Length Immunoglobulins in Escherichia coli: Rapid and Efficient Production of Aglycosylated Antibodies,” J. Immunol. Methods 263(1-2):133-147 (2002) and Rashid, M.H., “Full-Length Recombinant Antibodies from Escherichia coli: Production, Characterization, Effector Function (Fc) Engineering, and Clinical Evaluation,” Mabs. 14(1):2111748 (2022), which is hereby incorporated by reference in its entirety). This deficiency can be overcome by introducing specific mutations to the IgG Fc domain that confer FcγR binding (Jung et al., “Effective Phagocytosis of Low Her2 Tumor Cell Lines with Engineered, Aglycosylated IgG Displaying High FcγRIIa Affinity and Selectivity,” ACS Chem. Biol. 8(2):368-375 (2013); Jung et al., “Aglycosylated IgG Variants Expressed in Bacteria that Selectively bind FcgammaRI Potentiate Tumor Cell Killing by Monocyte-Dendritic Cells,” Proc. Natl. Acad. Sci. USA 107(2):604-609 (2010); and Kang et al., “An Engineered Human Fc Variant with Exquisite Selectivity for FcγRIIIa_{V158} Reveals that Ligation of FcγRIIIa Mediates Potent Antibody Dependent Cellular Phagocytosis with GM-CSF-Differentiated Macrophages,” Front. Immunol. 27:10:562 (2019), which are hereby incorporated by reference in their entirety), but all aglycosylated IgG mutants isolated so far exhibit selective binding to a single FcγR, which is in contrast to glycosylated IgGs derived from mammalian cells that bind all FcγRs. Hence, there remains great interest in combining Fc or IgG expression with protein glycosylation in E. coli.

Unfortunately, previous attempts to glycosylate Fc fragments in E. coli have largely been limited to attachment of bacterial N-glycans (Ollis et al., “Substitute Sweeteners: Diverse Bacterial Oligosaccharyltransferases with Unique N-Glycosylation Site Preferences,” Sci. Rep. 5:15237 (2015); Fisher et al., “Production of Secretory and Extracellular N-Linked Glycoproteins in Escherichia Coli,” Appl. Environ. Microbiol. 77(3):871-881 (2011); Schwarz et al., “A Combined Method for Producing Homogeneous Glycoproteins with Eukaryotic N-Glycosylation,” Nat. Chem. Biol. 6(4):264-266 (2010); and Schwarz et al., “Relaxed Acceptor Site Specificity of Bacterial Oligosaccharyltransferase In Vivo,” Glycobiology 21(1):45-54 (2011), which are insufficient to confer Fcγ receptor binding as shown here. While it is possible to attach eukaryotic N-glycans to the Fc domain using CjPglB in E. coli, this approach was met with inefficient glycosylation (approximately 1%) (Valderrama-Rincon et al., “An Engineered Eukaryotic Protein Glycosylation Pathway in Escherichia Coli,” Nat. Chem. Biol. 8(5):434-436 (2012), which is hereby incorporated by reference in its entirety).

The disclosed combined strategy overcomes the deficiencies of these previous works in two important ways. First, the use of DmPglB greatly increases the efficiency of Fc glycosylation including at the authentic QYNST (SEQ ID NO: 5) sequon and second, the chemoenzymatic remodeling strategy introduces eukaryotic complex-type glycans that permit the full spectrum of Fc effector functions that have until now been inaccessible to E. coli-derived IgGs. Although further improvements in glycosylation efficiency and yield will be required to rival IgG expression in mammalian host cell lines, the discovery of DmPglB provides a potent new N13 glycosylation catalyst to the bacterial glycoprotein engineering toolbox and creates an important foundation on which the production and glycoengineering of IgG antibodies and antibody fragments can be more deeply investigated and optimized in the future.

Although embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A recombinant oligosaccharyltransferase (OST) capable of catalyzing the transfer of a glycan onto a sequon comprising an N−X−T motif, wherein X can be any amino acid.

2. The recombinant oligosaccharyltransferase (OST) according to claim 1, wherein the sequon comprises an X₋₂QNX₋₁T (SEQ ID NO: 3) motif, where X₋₂ and X₋₁ can be any amino acid but proline, or an XQNAT (SEQ ID NO: 4) motif, wherein X can be any amino acid.

3. The recombinant oligosaccharyltransferase according to claim 1, wherein the sequon is selected from the group consisting of QYNST (SEQ ID NO: 5), DQNAT (SEQ ID NO:

6., AENIT (SEQ ID NO: 7), NENIT (SEQ ID NO: 8), LVNSS (SEQ ID NO: 9), SRNLT (SEQ ID NO: 10), QSNDT (SEQ ID NO: 11), FSNTT (SEQ ID NO: 12), PGNAS (SEQ ID NO: 13), QSNST (SEQ ID NO: 14), NFNLT (SEQ ID NO: 15), LGNAT (SEQ ID NO: 16), MENFS (SEQ ID NO:

17., SPNKT (SEQ ID NO: 18), DVNKS (SEQ ID NO: 19), LLNKS (SEQ ID NO: 20), SQNSS (SEQ ID NO: 21), and AQNAT (SEQ ID NO: 22).

4. The recombinant oligosaccharyltransferase according to claim 1, wherein the oligosaccharyltransferase is capable of catalyzing glycosylation of human IgG and/or fragments thereof.

5. The recombinant oligosaccharyltransferase according to claim 4, wherein the human IgG and/or fragments thereof comprise a C_{H}2 domains.

6. The recombinant oligosaccharyltransferase according to any one of the preceding claims, wherein the glycan is prokaryotic glycan.

7. The recombinant oligosaccharyltransferase according to claim 6, wherein the glycan is selected from the group consisting of GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAc₂ (Man5), Man₅₋₉GlcNAc₂(Man5-9 or high mannose glycan), GlcNAc₂Man₃GlcNAc₂ (G0), Gal₁GlcNAc₂Man₃GlcNAc₂ (G1), Gal₂GlcNAC₂Man₃GlcNAc₂ (G2), SiaGal₂GlcNAc₂Man₃GlcNAc₂ (S1G2), Sia₂Gal₂GlcNAc₂Man₃GlcNAc₂ (S2G2), GlcNAc₂Man₃GlcNAc₂(Fuc) (G0F), Gal₁GlcNAc₂Man₃GlcNAc₂(Fuc) (G1F), Gal₂GlcNAc₂Man₃GlcNAc₂(Fuc) (G2F), Sia₁Gal₂GlcNAc₂Man₃GlcNAc₂(Fuc) (S1G2F), Sia₂Gal₂GlcNAc₂Man₃GlcNAc₂ (Fuc) (S2G2F), mono-GlcNAc, bacterial capsular polysaccharide (CPS) antigens, and/or bacterial O-antigen polysaccharide (O-PS) antigens.

8. The recombinant oligosaccharyltransferase according to any one of claims 1 to 5, wherein the glycan is a eukaryotic glycan.

9. The recombinant oligosaccharyltransferase according to claim 8, wherein the glycan is selected from the group consisting of GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAc₂ (Man5), Man₅₋₉GlcNAc₂ (Man5-9 or high mannose glycan), GlcNAc₂Man₃GlcNAc₂ (G0), Gal₁GlcNAc₂Man₃GlcNAc₂ (G1), Gal₂GlcNAc₂Man₃GlcNAc₂ (G2), Sia₁Gal₂GlcNAc₂Man₃GlcNAc₂ (S1G2), Sia₂Gal₂GlcNAc₂Man₃GlcNAc₂ (S2G2), GlcNAc₂Man₃GlcNAC₂(Fuc) (G0F), Gal₁GlcNAc₂Man₃GlcNAC₂(Fuc) (G1F), Gal₂GlcNAc₂Man₃GlcNAc₂(Fuc) (G2F), Sia₁Gal₂GlcNAc₂Man₃GlcNAc₂(Fuc) (S1G2F), and/or Sia₂Gal₂GlcNAc₂Man₃GlcNAc₂ (Fuc) (S2G2F).

10. The recombinant oligosaccharyltransferase according to any one of the preceding claims, wherein the oligosaccharyltransferase is a single subunit OST.

11. The recombinant oligosaccharyltransferase according to any one of the preceding claims, wherein the oligosaccharyltransferase is a Desulfovibrio marinus oligosaccharyltransferase.

12. The recombinant oligosaccharyltransferase according to any one of the preceding claims, wherein the oligosaccharyltransferase is DmPglB.

13. The recombinant oligosaccharyltransferase according to any one of the preceding claims, wherein the oligosaccharyltransferase has the amino acid sequence of SEQ ID NO: 1:
MIFSREHSIRRDWKALIVTCVIVMLAGMAVRMQELPEWNQPAYRVAGEFIMGTHDAYHW
LAGAMGFGSAADAPPSELLRALSHMTGISVGNLGFFLPAIFGGLVGAATVLWAWALGGLE
AGLVAGVIATLAPGYYYRSRLGYYDTDIVTLLFPLLLTFGLAIWLDGSLCDSWVNRFRSAF
SKKNGKAVADATKDEGAEEETAAPDEPDEPRRFFLIWPALLGGFGSWAALWHGYMLTFL
QLTVFMLLFLVFVAGKRGRRGALLWGVAAFAAAGFWGLYGTLGAVVAALLAGALPKNIR
AKVYSLAPGLLAAAVVLVASGAAESIVVGGSKFLASYIKPVAQQTAFRGDTGELVFPGIGQ
SVIEAQNLPLAEVFDRFHPWGWLSLAGIGGFFMLLVLRPSALFLLPFLAIALSAVKLGTRMA
MFGAPAVGLGLGFLFLWIGRAVLGGQSWSRYVLTFILGALALGVALPGVSLFLTLPPTPVL
SRHHAQALIDLGKEADKSSEVWTWWDWGYATHYYAGLQSFADGGRHYGEHVFTLGLAL
TTPSPMQSAQLIQYSAEHNEEPWTEWEKMGLDKTRDFLRSLGTEDLHLKPPMPLYVVATF
ENIRLSPWICYYGTWDFEKEQGVHARVASIRESFNLDWEKGTMTFQDEKEPIEVKSIHVLSS
QGRKDRHYDKNTGPNLILNSESRRYYALDDLAFQSMLTQLLIAPKEFERLDRYFELVYDDF
PWVRVYKVREVPKDAPAKPQTPAVESPEANGTAANATQPTNGTESGENTTQPANTTQ,
or an amino acid sequence having at least 85%, 90%, 95%, 96%,
97%, 98%, or 99% sequence identity to SEQ ID NO: 1.

14. A nucleic acid molecule encoding the recombinant oligosaccharyltransferase according to any one of the preceding claims.

15. The nucleic acid sequence according to claim 14, wherein the nucleic acid sequence is SEQ ID NO: 2:
ATGATTTTTTCCCGTGAGCACTCTATCCGCCGTGATTGGAAAGCATTAAT
CGTAACTTGTGTGATTGTAATGCTGGCAGGTATGGCAGTGCGCATGCAGGAATTGCCC
GAGTGGAATCAACCAGCATACCGTGTAGCAGGTGAATTTATTATGGGCACACATGACG
CGTATCACTGGCTTGCAGGGGCGATGGGCTTCGGGTCAGCTGCTGACGCGCCGCCATCT
GAGTTGCTGCGTGCCCTGTCGCACATGACTGGGATCTCCGTGGGTAACCTTGGGTTCTT
TTTGCCTGCGATCTTCGGAGGCTTAGTTGGGGCGGCGACCGTCTTATGGGCCTGGGCCC
TTGGTGGTTTGGAGGCGGGCCTGGTGGCCGGTGTCATTGCCACGCTGGCGCCTGGTTAC
TACTACCGTTCACGTTTGGGGTACTATGACACAGATATCGTCACTCTGTTATTCCCATTG
CTTTTGACATTTGGGCTGGCGATCTGGTTGGATGGTAGCTTATGTGATAGTTGGGTGAA
CCGCTTTCGTTCGGCCTTTTCCAAGAAGAACGGAAAAGCTGTCGCTGATGCGACTAAG
GATGAAGGCGCGGAGGAGGAGACAGCCGCTCCAGACGAGCCCGATGAACCACGTCGT
TTCTTTTTAATCTGGCCTGCGTTGTTGGGAGGTTTCGGGTCCTGGGCAGCTCTGTGGCAT
GGTTACATGTTAACTTTCCTTCAGTTGACGGTGTTTATGTTGCTTTTTCTGGTATTCGTC
GCCGGTAAGCGCGGGCGCCGTGGAGCCTTATTGTGGGGAGTGGCCGCTTTCGCTGCGG
CCGGATTTTGGGGCTTATATGGCACGCTTGGGGCCGTAGTTGCCGCGCTTCTTGCGGGA
GCGCTTCCGAAGAACATCCGTGCCAAAGTGTATTCACTGGCTCCAGGGTTATTAGCAGC
TGCAGTTGTCTTGGTTGCTTCTGGGGCCGCGGAATCTATCGTTGTAGGTGGATCAAAGT
TTTTGGCTAGTTATATCAAGCCGGTGGCACAACAAACTGCCTTTCGTGGGGATACTGGT
GAACTGGTATTTCCTGGGATTGGGCAATCCGTTATTGAAGCACAGAACCTTCCATTAGC
TGAGGTCTTCGATCGTTTCCACCCATGGGGATGGCTTTCCCTGGCCGGTATCGGAGGTT
TTTTTATGTTACTGGTTCTGCGCCCGTCCGCTCTGTTTCTGCTTCCTTTCTTAGCCATTGC
ACTTTCCGCCGTTAAGTTAGGTACCCGCATGGCCATGTTTGGCGCCCCGGCGGTTGGGT
TGGGCCTTGGATTTTTATTCCTTTGGATCGGTCGTGCCGTGTTGGGTGGACAGAGCTGG
TCCCGTTATGTCCTGACGTTCATCCTTGGTGCCCTTGCGTTGGGGGTCGCGTTACCCGG
GGTAAGTTTATTCCTTACACTGCCGCCAACTCCCGTACTGTCGCGCCACCACGCGCAGG
CTTTGATTGACTTGGGCAAGGAGGCTGATAAATCATCGGAAGTGTGGACGTGGTGGGA
CTGGGGTTACGCGACGCACTACTACGCTGGACTTCAATCCTTCGCTGATGGGGGACGTC
ATTATGGCGAACACGTCTTTACTTTAGGGCTGGCATTGACAACGCCGAGTCCCATGCAA
AGCGCACAACTGATTCAGTATTCAGCGGAACACAACGAGGAGCCTTGGACCGAGTGGG
AGAAAATGGGCTTGGACAAGACCCGTGACTTCTTACGCTCTCTGGGAACTGAAGATCT
GCACTTAAAGCCTCCCATGCCACTTTATGTCGTGGCTACTTTTGAAAACATTCGTCTGTC
GCCTTGGATTTGTTATTATGGAACTTGGGACTTCGAGAAAGAGCAGGGTGTCCACGCG
CGTGTGGCGAGCATTCGCGAGAGTTTTAACTTGGACTGGGAAAAGGGAACGATGACTT
TTCAAGATGAAAAAGAACCCATTGAGGTCAAGTCGATCCATGTTTTGTCCTCGCAGGG
GCGCAAAGACCGTCATTATGATAAAAATACGGGCCCAAACCTTATCTTAAACAGCGAA
AGTCGCCGCTATTACGCGCTGGACGATTTGGCATTCCAATCAATGTTAACTCAGCTTCT
TATTGCCCCTAAGGAATTCGAACGTCTTGACCGCTATTTCGAATTAGTCTATGATGACT
TTCCGTGGGTCCGTGTATACAAGGTTCGCGAGGTACCGAAGGATGCGCCTGCTAAGCC
GCAGACACCGGCTGTCGAAAGTCCGGAAGCTAACGGCACTGCCGCAAATGCTACTCAA
CCAACTAATGGGACAGAATCCGGCGAGAACACCACCCAACCAGCTAACACGACACAG,
or a nucleic acid sequence having at least 85%, 90%, 95%,
96%, 97%, 98%, or 99% sequence identity to SEQ ID NO: 2.

16. A vector comprising:
a nucleic acid sequence encoding the recombinant oligosaccharyltransferase according to any one of claims 1 to 13 and
a promoter heterologous to the nucleic acid sequence encoding the recombinant oligosaccharyltransferase.

17. The vector according to claim 16, wherein the vector is a prokaryotic expression vector.

18. A host cell comprising the recombinant oligosaccharyltransferase according to any one of claims 1 to 13, the nucleic acid sequence according to claim 14 or claim 15, or the vector according to claim 16 or claim 17.

19. The host cell according to claim 18, wherein the host cell is a prokaryotic host cell.

20. The host cell according to claim 19, wherein the host cell is an Escherichia coli cell.

21. The host cell according to any one of claims 18 to 20 further comprising a protein of interest.

22. The host cell according to claim 21, wherein the protein of interest is selected from the group consisting of an antibody or derivative thereof including a fragment crystallizable (Fc) domain, a single-chain variable fragment (scFv), a single-chain antibody (scAb), a single-domain antibody (scAb), a Fab, V_{H}/V_{L} variable regions, a Fc domain (QYNST (SEQ ID NO: 5)), a human EPO (AENIT (SEQ ID NO: 7), NENIT (SEQ ID NO: 8), LVNSS (SEQ ID NO:) 9)), a Rnase A (SRNLT (SEQ ID NO: 10)), Fab domains (eg. Cetuximab, QSNDT (SEQ ID NO: 11), or Etanercept, FSNTT (SEQ ID NO: 12)/PGNAS (SEQ ID NO: 13)), Alpha-1-antitrypsin QSNST (SEQ ID NO: 14), NFNLT (SEQ ID NO: 15), LGNAT (SEQ ID NO: 16), CRM197 vaccine carrier MENFS (SEQ ID NO: 17), SPNKT (SEQ ID NO: 18), DVNKS (SEQ ID NO: 19), PD vaccine carrier LLNKS (SEQ ID NO: 20), and Murine Tnfa SQNSS (SEQ ID NO: 21).

23. The host cell according to claim 21 or claim 22, wherein the protein of interest is an antibody or an antigen-binding fragment thereof.

24. The host cell according to claim 23, wherein the antibody or antigen-binding fragment thereof is a human IgG or antigen-binding fragment thereof.

25. The host cell according to claim 24, wherein the human IgG or antigen-binding fragment thereof is of IgG1, IgG2, IgG3, or IgG4 isotype.

26. The host cell according to any one of claims 23 to 25, wherein the protein of interest is an antigen-binding fragment of human IgG, wherein the fragment is C_{H}2, C_{H}2-C_{H}3, hinge-C_{H}2, hinge-C_{H}2-C_{H}3, fragment crystallizable (Fc) domain, a single-chain variable fragment (scFv), single-chain antibody (scAb), single-domain antibody (scAb), Fab, and/or V_{H}/V_{L} variable regions.

27. A glycoprotein produced by the host cell according to any one of claims 18 to 26.

28. A method of producing a glycosylated protein, said method comprising:
providing a prokaryotic host cell expressing a heterologous prokaryotic oligosaccharyltransferase enzyme capable of transferring a glycan to an N-glycosylation acceptor site of a protein, said acceptor site comprising an N−X−T motif, wherein X can be any amino acid but proline, and
culturing the prokaryotic host cell under conditions effective to produce a glycosylated protein.

29. The method according to claim 28, wherein the N-glycosylation acceptor site of the protein comprises an X₋₂QNX₋₁T (SEQ ID NO: 3) motif, where X₋₂ and X₋₁ can be any amino acid but proline, or an XQNAT (SEQ ID NO: 4) motif, wherein X can be any amino acid.

30. The method according to claim 28, wherein the N-glycosylation acceptor site of the protein is selected from the group consisting of QYNST (SEQ ID NO: 5), DQNAT (SEQ ID NO: 6), AENIT (SEQ ID NO: 7), NENIT (SEQ ID NO: 8), LVNSS (SEQ ID NO: 9), SRNLT (SEQ ID NO: 10), QSNDT (SEQ ID NO: 11), FSNTT (SEQ ID NO: 12), PGNAS (SEQ ID NO: 13), QSNST (SEQ ID NO: 14), NFNLT (SEQ ID NO: 15), LGNAT (SEQ ID NO: 16), MENFS (SEQ ID NO: 17), SPNKT (SEQ ID NO: 18), DVNKS (SEQ ID NO: 19), LLNKS (SEQ ID NO: 20), SQNSS (SEQ ID NO: 21), and AQNAT (SEQ ID NO: 22).

31. The method according to claim 28, wherein the oligosaccharyltransferase is capable of catalyzing glycosylation of human IgG and/or an antigen-binding fragment thereof.

32. The method according to claim 31, wherein the human IgG and/or antigen-binding fragments thereof comprise a C_{H}2 domain.

33. The method according to any one of claims 28 to 32, wherein the glycan is prokaryotic glycan.

34. The method according to claim 33, wherein the glycan is selected from the group consisting of GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAc₂ (Man5), Man₅₋₉GlcNAC₂ (Man5-9 or high mannose glycan), GlcNAc₂Man₃GlcNAc₂ (G0), Gal₁GlcNAc₂Man₃GlcNAc₂ (G1), Gal₂GlcNAc₂Man₃GlcNAc₂ (G2), Sia₁Gal₂GlcNAc₂Man₃GlcNAc₂ (S1G2), Sia₂Gal₂GlcNAc₂Man₃GlcNAc₂ (S2G2), GlcNAc₂Man₃GlcNAC₂(Fuc) (G0F), Gal₁GlcNAc₂Man₃GlcNAc₂(Fuc) (G1F), Gal₂GlcNAc₂Man₃GlcNAc₂(Fuc) (G2F), Sia₁Gal₂GlcNAc₂Man₃GlcNAc₂(Fuc) (S1G2F), Sia₂Gal₂GlcNAc₂Man₃GlcNAc₂ (Fuc) (S2G2F), mono-GlcNAc, bacterial capsular polysaccharide (CPS) antigens, and/or bacterial O-antigen polysaccharide (O-PS) antigens.

35. The method according to any one of claims 28 to 32, wherein the glycan is a eukaryotic glycan.

36. The method according to claim 35, wherein the glycan is selected from the group consisting of GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAc₂ (Man5), Man₅₋₉GlcNAc₂ (Man5-9 or high mannose glycan), GlcNAc₂Man₃GlcNAc₂ (G0), Gal₁GlcNAc₂Man₃GlcNAc₂ (G1), Gal₂GlcNAc₂Man₃GlcNAc₂ (G2), Sia₁Gal₂GlcNAC₂Man₃GlcNAc₂ (S1G2), Sia₂Gal₂GlcNAc₂Man₃GlcNAc₂ (S2G2), GlcNAc₂Man₃GlcNAC₂(Fuc) (G0F), Gal₁GlcNAc₂Man₃GlcNAc₂(Fuc) (G1F), Gal₂GlcNAc₂Man₃GlcNAc₂(Fuc) (G2F), SiaGal₂GlcNAc₂Man₃GlcNAc₂(Fuc) (S1G2F), and/or Sia₂Gal₂GlcNAc₂Man₃GlcNAc₂ (Fuc) (S2G2F).

37. The method according to any one of claims 28 to 36, wherein the oligosaccharyltransferase is a single subunit OST.

38. The method according to any one of claims 28 to 37, wherein the oligosaccharyltransferase is a Desulfovibrio marinus oligosaccharyltransferase.

39. The method according to any one of claims 28 to 38, wherein the oligosaccharyltransferase is DmPglB.

40. The method according to any one of claims 28 to 39, wherein the oligosaccharyltransferase has the amino acid sequence of SEQ ID NO: 1:
MIFSREHSIRRDWKALIVTCVIVMLAGMAVRMQELPEWNQPAYRVAGEFI
MGTHDAYHWLAGAMGFGSAADAPPSELLRALSHMTGISVGNLGFFLPAIF
GGLVGAATVLWAWALGGLEAGLVAGVIATLAPGYYYRSRLGYYDTDIVTL
LFPLLLTFGLAIWLDGSLCDSWVNRFRSAFSKKNGKAVADATKDEGAEEE
TAAPDEPDEPRRFFLIWPALLGGFGSWAALWHGYMLTFLQLTVFMLLFLV
FVAGKRGRRGALLWGVAAFAAAGFWGLYGTLGAVVAALLAGALPKNIRAK
VYSLAPGLLAAAVVLVASGAAESIVVGGSKFLASYIKPVAQQTAFRGDTG
ELVFPGIGQSVIEAQNLPLAEVFDRFHPWGWLSLAGIGGFFMLLVLRPSA
LFLLPFLAIALSAVKLGTRMAMFGAPAVGLGLGFLFLWIGRAVLGGQSWS
RYVLTFILGALALGVALPGVSLFLTLPPTPVLSRHHAQALIDLGKEADKS
SEVWTWWDWGYATHYYAGLQSFADGGRHYGEHVFTLGLALTTPSPMQSAQ
LIQYSAEHNEEPWTEWEKMGLDKTRDFLRSLGTEDLHLKPPMPLYVVATF
ENIRLSPWICYYGTWDFEKEQGVHARVASIRESFNLDWEKGTMTFQDEKE
PIEVKSIHVLSSQGRKDRHYDKNTGPNLILNSESRRYYALDDLAFQSMLT
QLLIAPKEFERLDRYFELVYDDFPWVRVYKVREVPKDAPAKPQTPAVESP
EANGTAANATQPTNGTESGENTTQPANTTQ,
or an amino acid sequence having at least 85%,
90%, 95%, 96%, 97%, 98%, or 99% sequence identity
to SEQ ID NO: 1.

41. The method according to any one of claims 28 to 40, wherein the prokaryotic host cell is selected from the group consisting of E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix, sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp, Stenotrophomonas sp., Micrococcus sp., Neisseria sp., Bdellovibrio sp., Hemophilus sp., Klebsiella sp., Proteus mirabilis, Enterobacter cloacae, Serratia sp., Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderia sp., Aeromonas sp., Plesiomonas sp., Legionella sp. and alpha-proteobacteria such as Wolbachia sp., cyanobacteria, spirochaetes, green sulfur and green non-sulfur bacteria, Gram-negative cocci, Gram negative bacilli which are fastidious, Enterobacteriaceae-glucose-fermenting gram-negative bacilli, Gram negative bacilli-non-glucose fermenters, Gram negative bacilli-glucose fermenting, oxidase positive.

42. The method according to any one of claims 28 to 41, wherein the prokaryotic host cell is an E. coli host cell.

43. The method according to claim 42, wherein the E. coli host cell is E. coli strain CLM24, JUDE-1, BL21 (DE3), a variant of BL21, SHuffle and all of its variations, CyDisCo and derivatives, FÅ113 and derivatives, Origami and derivatives, BW25113 and derivatives, MG1655 and derivatives, W3110 and derivatives, AF1000 and derivatives, Rosetta and derivatives, Rosetta-gami B strains, KS272 and derivatives, Lemo21(DE3), NiCo21(DE3), Tuner(DE3), BLR (DE3), or KRX.

44. The method according to any one of claims 28 to 43, wherein the host cell does not comprise a native oligosaccharyltransferase activity.

45. The method according to any one of claims 28 to 44, wherein the heterologous oligosaccharyltransferase enzyme is encoded by a nucleic acid molecule comprising the nucleotide sequence of SEQ ID NO: 2:
ATGATTTTTTCCCGTGAGCACTCTATCCGCCGTGATTGGAAAGCATTAATCGTAACTTG
TGTGATTGTAATGCTGGCAGGTATGGCAGTGCGCATGCAGGAATTGCCCGAGTGGAAT
CAACCAGCATACCGTGTAGCAGGTGAATTTATTATGGGCACACATGACGCGTATCACT
GGCTTGCAGGGGCGATGGGCTTCGGGTCAGCTGCTGACGCGCCGCCATCTGAGTTGCT
GCGTGCCCTGTCGCACATGACTGGGATCTCCGTGGGTAACCTTGGGTTCTTTTTGCCTG
CGATCTTCGGAGGCTTAGTTGGGGCGGCGACCGTCTTATGGGCCTGGGCCCTTGGTGGT
TTGGAGGCGGGCCTGGTGGCCGGTGTCATTGCCACGCTGGCGCCTGGTTACTACTACCG
TTCACGTTTGGGGTACTATGACACAGATATCGTCACTCTGTTATTCCCATTGCTTTTGAC
ATTTGGGCTGGCGATCTGGTTGGATGGTAGCTTATGTGATAGTTGGGTGAACCGCTTTC
GTTCGGCCTTTTCCAAGAAGAACGGAAAAGCTGTCGCTGATGCGACTAAGGATGAAGG
CGCGGAGGAGGAGACAGCCGCTCCAGACGAGCCCGATGAACCACGTCGTTTCTTTTTA
ATCTGGCCTGCGTTGTTGGGAGGTTTCGGGTCCTGGGCAGCTCTGTGGCATGGTTACAT
GTTAACTTTCCTTCAGTTGACGGTGTTTATGTTGCTTTTTCTGGTATTCGTCGCCGGTAA
GCGCGGGCGCCGTGGAGCCTTATTGTGGGGAGTGGCCGCTTTCGCTGCGGCCGGATTTT
GGGGCTTATATGGCACGCTTGGGGCCGTAGTTGCCGCGCTTCTTGCGGGAGCGCTTCCG
AAGAACATCCGTGCCAAAGTGTATTCACTGGCTCCAGGGTTATTAGCAGCTGCAGTTGT
CTTGGTTGCTTCTGGGGCCGCGGAATCTATCGTTGTAGGTGGATCAAAGTTTTTGGCTA
GTTATATCAAGCCGGTGGCACAACAAACTGCCTTTCGTGGGGATACTGGTGAACTGGT
ATTTCCTGGGATTGGGCAATCCGTTATTGAAGCACAGAACCTTCCATTAGCTGAGGTCT
TCGATCGTTTCCACCCATGGGGATGGCTTTCCCTGGCCGGTATCGGAGGTTTTTTTATGT
TACTGGTTCTGCGCCCGTCCGCTCTGTTTCTGCTTCCTTTCTTAGCCATTGCACTTTCCGC
CGTTAAGTTAGGTACCCGCATGGCCATGTTTGGCGCCCCGGCGGTTGGGTTGGGCCTTG
GATTTTTATTCCTTTGGATCGGTCGTGCCGTGTTGGGTGGACAGAGCTGGTCCCGTTAT
GTCCTGACGTTCATCCTTGGTGCCCTTGCGTTGGGGGTCGCGTTACCCGGGGTAAGTTT
ATTCCTTACACTGCCGCCAACTCCCGTACTGTCGCGCCACCACGCGCAGGCTTTGATTG
ACTTGGGCAAGGAGGCTGATAAATCATCGGAAGTGTGGACGTGGTGGGACTGGGGTTA
CGCGACGCACTACTACGCTGGACTTCAATCCTTCGCTGATGGGGGACGTCATTATGGCG
AACACGTCTTTACTTTAGGGCTGGCATTGACAACGCCGAGTCCCATGCAAAGCGCACA
ACTGATTCAGTATTCAGCGGAACACAACGAGGAGCCTTGGACCGAGTGGGAGAAAATG
GGCTTGGACAAGACCCGTGACTTCTTACGCTCTCTGGGAACTGAAGATCTGCACTTAAA
GCCTCCCATGCCACTTTATGTCGTGGCTACTTTTGAAAACATTCGTCTGTCGCCTTGGAT
TTGTTATTATGGAACTTGGGACTTCGAGAAAGAGCAGGGTGTCCACGCGCGTGTGGCG
AGCATTCGCGAGAGTTTTAACTTGGACTGGGAAAAGGGAACGATGACTTTTCAAGATG
AAAAAGAACCCATTGAGGTCAAGTCGATCCATGTTTTGTCCTCGCAGGGGCGCAAAGA
CCGTCATTATGATAAAAATACGGGCCCAAACCTTATCTTAAACAGCGAAAGTCGCCGC
TATTACGCGCTGGACGATTTGGCATTCCAATCAATGTTAACTCAGCTTCTTATTGCCCCT
AAGGAATTCGAACGTCTTGACCGCTATTTCGAATTAGTCTATGATGACTTTCCGTGGGT
CCGTGTATACAAGGTTCGCGAGGTACCGAAGGATGCGCCTGCTAAGCCGCAGACACCG
GCTGTCGAAAGTCCGGAAGCTAACGGCACTGCCGCAAATGCTACTCAACCAACTAATG
GGACAGAATCCGGCGAGAACACCACCCAACCAGCTAACACGACACAG,
or a nucleic acid sequence having at least 85%, 90%, 95%,
96%, 97%, 98%, or 99% sequence identity to SEQ ID NO: 2.

46. The method according to any one of claims 28 to 45, wherein the prokaryotic host cell further express a heterologous protein of interest.

47. The method according to claim 46, wherein the protein of interest is selected from the group consisting of an antibody, an antibody, a monoclonal IgG1 antibody or derivative thereof including fragment crystallizable (Fc) domain, a single-chain variable fragment (scFv), a single-chain antibody (scAb), a single-domain antibody (scAb), a Fab, V_{H}/V_{L} variable regions, a Fc domain (QYNST (SEQ ID NO: 5)), a human EPO (AENIT (SEQ ID NO: 7), NENIT (SEQ ID NO: 8), LVNSS (SEQ ID NO: 9)), a Rnase A (SRNLT (SEQ ID NO: 10)), Fab domains (eg. Cetuximab, QSNDT (SEQ ID NO: 11), or Etanercept, FSNTT (SEQ ID NO: 12)/PGNAS (SEQ ID NO: 13)), Alpha-1-antitrypsin QSNST (SEQ ID NO: 14), NFNLT (SEQ ID NO: 15), LGNAT (SEQ ID NO: 16), CRM197 vaccine carrier MENFS (SEQ ID NO: 17), SPNKT (SEQ ID NO: 18), DVNKS (SEQ ID NO: 19), PD vaccine carrier LLNKS (SEQ ID NO: 20), and Murine Tnfa SQNSS (SEQ ID NO: 21.

48. The method according to claim 47, wherein the protein of interest is an antibody or an antigen-binding fragment thereof.

49. The method according to claim 48, wherein the antibody or antigen-binding fragment thereof is a human IgG or fragment thereof.

50. The method according to claim 49, wherein the human IgG or fragment thereof is of IgG1, IgG2, IgG3, or IgG4 isotype.

51. The method according to any one of claims 47 to 50, wherein the protein of interest is a fragment of human IgG, wherein the fragment is C_{H}2, C_{H}2-C_{H}3, hinge-C_{H}2, hinge-C_{H}2-C_{H}3, fragment crystallizable (Fc) domain, single-chain variable fragment (scFv), single-chain antibody (scAb), single-domain antibody (scAb), Fab, and/or V_{H}/V_{L} variable regions.

52. The method according to any one of claims 47 to 51, wherein the protein of interest is selected from the group consisting of scFv13, YebF, RNase A, hinge-Fc, and full-length IgG.

53. The method according to any one of claims 28 to 52, wherein the prokaryotic host cell lacks a native glycosylation pathway.

54. The method according to any one of claims 28 to 53, wherein the prokaryotic host cell further expresses a heterologous glycosylation pathway.

55. The method according to any one of claims 28 to 54, wherein the prokaryotic host cell further expresses GalNac₅GlcNAc, GalNAc₅(Glc)GlcNAc, GalNAc₅GlcNAc, GlcNAcGlcNAc (diGlcNAc or chitobiose), mono-GlcNAc, SiaGalGlcNAc, Man₃GlcNAc₂ (Man3 or trimmanosyl core glycan), Man₅GlcNAC₂ (Man5), Man₅₋₉GlcNAc₂ (Man5-9 or high mannose glycan), GlcNAc₂Man₃GlcNAc₂ (G0), Gal₁GlcNAc₂Man₃GlcNAc₂ (G1), Gal₂GlcNAc₂Man₃GlcNAc₂ (G2), Sia₁Gal₂GlcNAC₂Man₃GlcNAc₂ (S1G2), Sia₂Gal₂GlcNAc₂Man₃GlcNAc₂ (S2G2), GlcNAc₂Man₃GlcNAc₂(Fuc) (G0F), Gal₁GlcNAc₂Man₃GlcNAc₂(Fuc) (G1F), Gal₂GlcNAC₂Man₃GlcNAc₂(Fuc) (G2F), Sia₁Gal₂GlcNAc₂Man₃GlcNAc₂(Fuc) (S1G2F), Sia₂Gal₂GlcNAc₂Man₃GlcNAc₂ (Fuc) (S2G2F), mono-GlcNAc, bacterial capsular polysaccharide (CPS) antigens, and/or bacterial O-antigen polysaccharide (O-PS) antigens.

56. The method according to any one of claims 28 to 55, wherein the glycosylated protein comprises an N-linked GalNac₅GlcNAc.

57. The method according to claim 56, wherein the method further comprises:
removing GalNAc from the N-linked GalNac₅GlcNAc.

58. The method according to claim 57, wherein said removing comprises subjecting the glycosylated protein to enzymatic trimming with an exo-α-N-acetylglycosamineidase to form a GlcNAc stump.

59. The method according to claim 58, further comprising:
transglycosylating the GlcNAc stump.

60. The method according to claim 59, wherein said transglycosylating is catalyzed by EndoS2-D184M with a G2-oxaxoline as a donor substrate to produce a glycosylated protein comprising Gal₂GlcNAc₂Man₃GlcNAc₂, EndoF3 D165A, Endo-S D233Q, Endo-CC1 N180H, and/or Endo-M N175Q.

61. A system comprising:
a first plasmid encoding enzymes for N-glycan biosynthesis;
a second plasmid encoding a recombinant oligosaccharyltransferase (OST) according to any one of claims 1-15; and/or
a third plasmid encoding a protein of interest.

62. The system according to claim 61, wherein the protein of interest is a glycoprotein target.

63. The system according to claim 61 or claim 62, wherein the protein of interest is selected from the group consisting of an antibody, a monoclonal IgG1 antibody or derivative thereof including fragment crystallizable (Fc) domain, a single-chain variable fragment (scFv), a single-chain antibody (scAb), a single-domain antibody (scAb), a Fab, V_{H}/V_{L} variable regions, a Fc domain (QYNST (SEQ ID NO: 5)), a human EPO (AENIT (SEQ ID NO: 7), NENIT (SEQ ID NO: 8), LVNSS (SEQ ID NO: 9)), a Rnase A (SRNLT (SEQ ID NO: 10)), Fab domains (eg. Cetuximab, QSNDT (SEQ ID NO: 11), or Etanercept, FSNTT (SEQ ID NO: 12)/PGNAS (SEQ ID NO: 13)), Alpha-1-antitrypsin QSNST (SEQ ID NO: 14), NFNLT (SEQ ID NO: 15), LGNAT (SEQ ID NO: 16), CRM197 vaccine carrier MENFS (SEQ ID NO: 17), SPNKT (SEQ ID NO: 18), DVNKS (SEQ ID NO: 19), PD vaccine carrier LLNKS (SEQ ID NO: 20), and Murine Tnfa SQNSS (SEQ ID NO: 21).

64. The system according to claim 63, wherein the protein of interest is an antibody or an antigen-binding fragment thereof.

65. The system according to claim 64, wherein the antibody or antigen-binding fragment thereof is a human IgG or fragment thereof.

66. The system according to claim 65, wherein the human IgG or antigen-binding fragment thereof is of IgG1, IgG2, IgG3, or IgG4 isotype.

67. The system according to any one of claims 61 to 66, wherein the protein of interest is a fragment of human IgG, wherein the fragment is C_{H}2, C_{H}2-C_{H}3, hinge-C_{H}2, hinge-C₂2-C_{H}3, fragment crystallizable (Fc) domain, a single-chain variable fragment (scFv), single-chain antibody (scAb), single-domain antibody (scAb), Fab, and/or V_{H}/V_{L} variable regions.

68. The system according to any one of claims 61 to 67, wherein the protein of interest is selected from the group consisting of scFv13, YebF, RNase A, hinge-Fc, and full-length IgG.

69. The system according to any one of claims 61 to 67, wherein the protein of interest comprises a natural N-glycan acceptor site.

70. The system according to any one of claims 61 to 67, wherein the protein of interest comprises an engineered N-glycan acceptor site.

71. The system according to any one of claims 61 to 70, wherein the protein of interest comprises an N−X−T motif, wherein X can be any amino acid.

72. The system according to claim 71, wherein the protein of interest comprises an X₋₂QNX₋₁T (SEQ ID NO: 3) motif, where X₋₂ and X₋₁ can be any amino acid but proline, or an XQNAT (SEQ ID NO: 4) motif, wherein X can be any amino acid.

73. The system according to claim 71, wherein the protein of interest comprises a sequon selected from the group consisting of QYNST (SEQ ID NO: 5), DQNAT (SEQ ID NO: 6), AENIT (SEQ ID NO: 7), NENIT (SEQ ID NO: 8), LVNSS (SEQ ID NO: 9), SRNLT (SEQ ID NO: 10), QSNDT (SEQ ID NO: 11), FSNTT (SEQ ID NO: 12), PGNAS (SEQ ID NO: 13), QSNST (SEQ ID NO: 14), NFNLT (SEQ ID NO: 15), LGNAT (SEQ ID NO: 16), MENFS (SEQ ID NO: 17), SPNKT (SEQ ID NO: 18), DVNKS (SEQ ID NO: 19), LINKS (SEQ ID NO: 20), SQNSS (SEQ ID NO: 21), and AQNAT (SEQ ID NO: 22).


SEQUENCE LISTING (229 sequences)

SEQ ID NO: 1 - protein, 780 aa, synthetic construct
MIFSREHSIRRDWKALIVTCVIVMLAGMAVRMQELPEWNQPAYRVAGEFIMGTHDAYHWLAGAMGFGSAADAPPSELLRALSHMTGISVGNLGFFLPAIFGGLVGAATVLWAWALGGLEAGLVAGVIATLAPGYYYRSRLGYYDTDIVTLLFPLLLTFGLAIWLDGSLCDSWVNRFRSAFSKKNGKAVADATKDEGAEEETAAPDEPDEPRRFFLIWPALLGGFGSWAALWHGYMLTFLQLTVFMLLFLVFVAGKRGRRGALLWGVAAFAAAGFWGLYGTLGAVVAALLAGALPKNIRAKVYSLAPGLLAAAVVLVASGAAESIVVGGSKFLASYIKPVAQQTAFRGDTGELVFPGIGQSVIEAQNLPLAEVFDRFHPWGWLSLAGIGGFFMLLVLRPSALFLLPFLAIALSAVKLGTRMAMFGAPAVGLGLGFLFLWIGRAVLGGQSWSRYVLTFILGALALGVALPGVSLFLTLPPTPVLSRHHAQALIDLGKEADKSSEVWTWWDWGYATHYYAGLQSFADGGRHYGEHVFTLGLALTTPSPMQSAQLIQYSAEHNEEPWTEWEKMGLDKTRDFLRSLGTEDLHLKPPMPLYVVATFENIRLSPWICYYGTWDFEKEQGVHARVASIRESFNLDWEKGTMTFQDEKEPIEVKSIHVLSSQGRKDRHYDKNTGPNLILNSESRRYYALDDLAFQSMLTQLLIAPKEFERLDRYFELVYDDFPWVRVYKVREVPKDAPAKPQTPAVESPEANGTAANATQPTNGTESGENTTQPANTTQ

SEQ ID NO: 2 - DNA, 2340 bp, synthetic construct
  source 1..2340: mol_type = other DNA
atgattttttcccgtgagcactctatccgccgtgattggaaagcattaatcgtaacttgtgtgattgtaatgctggcaggtatggcagtgcgcatgcaggaattgcccgagtggaatcaaccagcataccgtgtagcaggtgaatttattatgggcacacatgacgcgtatcactggcttgcaggggcgatgggcttcgggtcagctgctgacgcgccgccatctgagttgctgcgtgccctgtcgcacatgactgggatctccgtgggtaaccttgggttctttttgcctgcgatcttcggaggcttagttggggcggcgaccgtcttatgggcctgggcccttggtggtttggaggcgggcctggtggccggtgtcattgccacgctggcgcctggttactactaccgttcacgtttggggtactatgacacagatatcgtcactctgttattcccattgcttttgacatttgggctggcgatctggttggatggtagcttatgtgatagttgggtgaaccgctttcgttcggccttttccaagaagaacggaaaagctgtcgctgatgcgactaaggatgaaggcgcggaggaggagacagccgctccagacgagcccgatgaaccacgtcgtttctttttaatctggcctgcgttgttgggaggtttcgggtcctgggcagctctgtggcatggttacatgttaactttccttcagttgacggtgtttatgttgctttttctggtattcgtcgccggtaagcgcgggcgccgtggagccttattgtggggagtggccgctttcgctgcggccggattttggggcttatatggcacgcttggggccgtagttgccgcgcttcttgcgggagcgcttccgaagaacatccgtgccaaagtgtattcactggctccagggttattagcagctgcagttgtcttggttgcttctggggccgcggaatctatcgttgtaggtggatcaaagtttttggctagttatatcaagccggtggcacaacaaactgcctttcgtggggatactggtgaactggtatttcctgggattgggcaatccgttattgaagcacagaaccttccattagctgaggtcttcgatcgtttccacccatggggatggctttccctggccggtatcggaggtttttttatgttactggttctgcgcccgtccgctctgtttctgcttcctttcttagccattgcactttccgccgttaagttaggtacccgcatggccatgtttggcgccccggcggttgggttgggccttggatttttattcctttggatcggtcgtgccgtgttgggtggacagagctggtcccgttatgtcctgacgttcatccttggtgcccttgcgttgggggtcgcgttacccggggtaagtttattccttacactgccgccaactcccgtactgtcgcgccaccacgcgcaggctttgattgacttgggcaaggaggctgataaatcatcggaagtgtggacgtggtgggactggggttacgcgacgcactactacgctggacttcaatccttcgctgatgggggacgtcattatggcgaacacgtctttactttagggctggcattgacaacgccgagtcccatgcaaagcgcacaactgattcagtattcagcggaacacaacgaggagccttggaccgagtgggagaaaatgggcttggacaagacccgtgacttcttacgctctctgggaactgaagatctgcacttaaagcctcccatgccactttatgtcgtggctacttttgaaaacattcgtctgtcgccttggatttgttattatggaacttgggacttcgagaaagagcagggtgtccacgcgcgtgtggcgagcattcgcgagagttttaacttggactgggaaaagggaacgatgacttttcaagatgaaaaagaacccattgaggtcaagtcgatccatgttttgtcctcgcaggggcgcaaagaccgtcattatgataaaaatacgggcccaaaccttatcttaaacagcgaaagtcgccgctattacgcgctggacgatttggcattccaatcaatgttaactcagcttcttattgcccctaaggaattcgaacgtcttgaccgctatttcgaattagtctatgatgactttccgtgggtccgtgtatacaaggttcgcgaggtaccgaaggatgcgcctgctaagccgcagacaccggctgtcgaaagtccggaagctaacggcactgccgcaaatgctactcaaccaactaatgggacagaatccggcgagaacaccacccaaccagctaacacgacacag

SEQ ID NO: 3 - skipped

SEQ ID NO: 4 - protein, 5 aa, synthetic construct
  VARIANT 1: note = X at position 1 can be any amino acid
XQNAT

SEQ ID NO: 5 - protein, 5 aa, synthetic construct
QYNST

SEQ ID NO: 6 - protein, 5 aa, synthetic construct
DQNAT

SEQ ID NO: 7 - protein, 5 aa, synthetic construct
AENIT

SEQ ID NO: 8 - protein, 5 aa, synthetic construct
NENIT

SEQ ID NO: 9 - protein, 5 aa, synthetic construct
LVNSS

SEQ ID NO: 10 - protein, 5 aa, synthetic construct
SRNLT

SEQ ID NO: 11 - protein, 5 aa, synthetic construct
QSNDT

SEQ ID NO: 12 - protein, 5 aa, synthetic construct
FSNTT

SEQ ID NO: 13 - protein, 5 aa, synthetic construct
PGNAS

SEQ ID NO: 14 - protein, 5 aa, synthetic construct
QSNST

SEQ ID NO: 15 - protein, 5 aa, synthetic construct
NFNLT

SEQ ID NO: 16 - protein, 5 aa, synthetic construct
LGNAT

SEQ ID NO: 17 - protein, 5 aa, synthetic construct
MENFS

SEQ ID NO: 18 - protein, 5 aa, synthetic construct
SPNKT

SEQ ID NO: 19 - protein, 5 aa, synthetic construct
DVNKS

SEQ ID NO: 20 - protein, 5 aa, synthetic construct
LLNKS

SEQ ID NO: 21 - protein, 5 aa, synthetic construct
SQNSS

SEQ ID NO: 22 - protein, 5 aa, synthetic construct
AQNAT

SEQ ID NO: 23 - protein, 5 aa, synthetic construct
  VARIANT 4: note = X at position 4 is Y or W
WWDXG

SEQ ID NO: 24 - protein, 5 aa, synthetic construct
EANGT

SEQ ID NO: 25 - protein, 5 aa, synthetic construct
AANAT

SEQ ID NO: 26 - protein, 5 aa, synthetic construct
FSNYS

SEQ ID NO: 27 - protein, 5 aa, synthetic construct
RDNAT

SEQ ID NO: 28 - protein, 5 aa, synthetic construct
  VARIANT 1: note = X at position 1 is D or A
XQNAT

SEQ ID NO: 29 - protein, 5 aa, synthetic construct
WWDWG

SEQ ID NO: 30 - protein, 5 aa, synthetic construct
WWDYG

SEQ ID NO: 31 - protein, 4 aa, synthetic construct
DGGR

SEQ ID NO: 32 - protein, 4 aa, synthetic construct
DGGK

SEQ ID NO: 33 - skipped

SEQ ID NO: 34 - protein, 4 aa, synthetic construct
SVSE

SEQ ID NO: 35 - protein, 4 aa, synthetic construct
SVIE

SEQ ID NO: 36 - protein, 4 aa, synthetic construct
SIIE

SEQ ID NO: 37-39 - skipped (3 entries)

SEQ ID NO: 40 - protein, 9 aa, synthetic construct
EEQYNSTYR

SEQ ID NO: 41 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 42 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPEL

SEQ ID NO: 43 - protein, 125 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 44 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPEL

SEQ ID NO: 45 - protein, 109 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 46 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPEL

SEQ ID NO: 47 - protein, 121 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 48 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPEL

SEQ ID NO: 49 - protein, 110 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 50 - protein, 216 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPE

SEQ ID NO: 51 - protein, 172 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 52 - protein, 278 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPE

SEQ ID NO: 53 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 54 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLEL

SEQ ID NO: 55 - protein, 122 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 56 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLEL

SEQ ID NO: 57 - protein, 8 aa, synthetic construct
ALEGGQYN

SEQ ID NO: 58 - protein, 5 aa, synthetic construct
PTNGT

SEQ ID NO: 59 - protein, 5 aa, synthetic construct
GENTT

SEQ ID NO: 60 - protein, 5 aa, synthetic construct
PANTT

SEQ ID NO: 61 - skipped

SEQ ID NO: 62 - protein, 8 aa, synthetic construct
  VARIANT 3: note = X at position 3 can be any amino acid
  VARIANT 5: note = X at position 5 can be any amino acid
  VARIANT 7: note = X at position 7 can be any amino acid
  VARIANT 8: note = X at position 8 can be T or S
DNXTXNXX

SEQ ID NO: 63 - skipped

SEQ ID NO: 64 - protein, 6 aa, synthetic construct
DQNATF

SEQ ID NO: 65 - protein, 8 aa, synthetic construct
  VARIANT 1: note = X at position 1 is modified with TAMRA
  VARIANT 8: note = X at position 8 is modified with NH2
GSDQNATF

SEQ ID NO: 66 - protein, 8 aa, synthetic construct
  VARIANT 1: note = X at position 1 is modified with TAMRA
  VARIANT 8: note = X at position 8 is modified with NH2
GQYNSTAF

SEQ ID NO: 67 - protein, 5 aa, synthetic construct
ADNST

SEQ ID NO: 68 - protein, 5 aa, synthetic construct
ARNET

SEQ ID NO: 69 - protein, 5 aa, synthetic construct
ASNCT

SEQ ID NO: 70 - protein, 5 aa, synthetic construct
ASNST

SEQ ID NO: 71 - protein, 5 aa, synthetic construct
CCNMT

SEQ ID NO: 72 - protein, 5 aa, synthetic construct
CGNNT

SEQ ID NO: 73 - protein, 5 aa, synthetic construct
CHNST

SEQ ID NO: 74 - protein, 5 aa, synthetic construct
CQNST

SEQ ID NO: 75 - protein, 5 aa, synthetic construct
DRNKT

SEQ ID NO: 76 - protein, 5 aa, synthetic construct
DTNGT

SEQ ID NO: 77 - protein, 5 aa, synthetic construct
EKNMT

SEQ ID NO: 78 - protein, 5 aa, synthetic construct
EPNYT

SEQ ID NO: 79 - protein, 5 aa, synthetic construct
EQNAT

SEQ ID NO: 80 - protein, 5 aa, synthetic construct
ESNGT

SEQ ID NO: 81 - protein, 5 aa, synthetic construct
FGNST

SEQ ID NO: 82 - protein, 5 aa, synthetic construct
FPNGT

SEQ ID NO: 83 - protein, 5 aa, synthetic construct
GRNAT

SEQ ID NO: 84 - protein, 5 aa, synthetic construct
GQNAT

SEQ ID NO: 85 - protein, 5 aa, synthetic construct
NHNCT

SEQ ID NO: 86 - protein, 5 aa, synthetic construct
NTNAT

SEQ ID NO: 87 - protein, 5 aa, synthetic construct
PANAT

SEQ ID NO: 88 - protein, 5 aa, synthetic construct
PNNAT

SEQ ID NO: 89 - protein, 5 aa, synthetic construct
PNNRT

SEQ ID NO: 90 - protein, 5 aa, synthetic construct
PQNAT

SEQ ID NO: 91 - protein, 5 aa, synthetic construct
PQNST

SEQ ID NO: 92 - protein, 5 aa, synthetic construct
PSNST

SEQ ID NO: 93 - protein, 5 aa, synthetic construct
PTNST

SEQ ID NO: 94 - protein, 5 aa, synthetic construct
PVNAT

SEQ ID NO: 95 - protein, 5 aa, synthetic construct
QHNST

SEQ ID NO: 96 - protein, 5 aa, synthetic construct
QMNAT

SEQ ID NO: 97 - protein, 5 aa, synthetic construct
QQNST

SEQ ID NO: 98 - protein, 5 aa, synthetic construct
QTNGT

SEQ ID NO: 99 - protein, 5 aa, synthetic construct
SHNAT

SEQ ID NO: 100 - protein, 5 aa, synthetic construct
SHNNT

SEQ ID NO: 101 - protein, 5 aa, synthetic construct
SQNST

SEQ ID NO: 102 - protein, 5 aa, synthetic construct
SSNCT

SEQ ID NO: 103 - protein, 5 aa, synthetic construct
HANST

SEQ ID NO: 104 - protein, 5 aa, synthetic construct
HQNAT

SEQ ID NO: 105 - protein, 5 aa, synthetic construct
HSNST

SEQ ID NO: 106 - protein, 5 aa, synthetic construct
HTNST

SEQ ID NO: 107 - protein, 5 aa, synthetic construct
HYNAT

SEQ ID NO: 108 - protein, 5 aa, synthetic construct
KSNDT

SEQ ID NO: 109 - protein, 5 aa, synthetic construct
MGNAT

SEQ ID NO: 110 - protein, 5 aa, synthetic construct
MTNAT

SEQ ID NO: 111 - protein, 5 aa, synthetic construct
MTNST

SEQ ID NO: 112 - protein, 5 aa, synthetic construct
NANAT

SEQ ID NO: 113 - protein, 5 aa, synthetic construct
NCNCT

SEQ ID NO: 114 - protein, 5 aa, synthetic construct
NDNCT

SEQ ID NO: 115 - protein, 5 aa, synthetic construct
STNAT

SEQ ID NO: 116 - protein, 5 aa, synthetic construct
STNST

SEQ ID NO: 117 - protein, 5 aa, synthetic construct
SVNAT

SEQ ID NO: 118 - protein, 5 aa, synthetic construct
TFNAT

SEQ ID NO: 119 - protein, 5 aa, synthetic construct
TNNAT

SEQ ID NO: 120 - protein, 5 aa, synthetic construct
TSNAT

SEQ ID NO: 121 - protein, 5 aa, synthetic construct
TTNST

SEQ ID NO: 122 - protein, 5 aa, synthetic construct
TYNAT

SEQ ID NO: 123 - protein, 5 aa, synthetic construct
VANHT

SEQ ID NO: 124 - protein, 5 aa, synthetic construct
WSNST

SEQ ID NO: 125 - protein, 5 aa, synthetic construct
YTNAT

SEQ ID NO: 126 - protein, 9 aa, synthetic construct
DNNTWNNTH

SEQ ID NO: 127 - protein, 9 aa, synthetic construct
DNNTWNNSH

SEQ ID NO: 128 - protein, 9 aa, synthetic construct
DGNTWNHEH

SEQ ID NO: 129 - protein, 9 aa, synthetic construct
DGNTWSHKH

SEQ ID NO: 130 - protein, 5 aa, synthetic construct
DGGKH

SEQ ID NO: 131 - protein, 5 aa, synthetic construct
DGASH

SEQ ID NO: 132 - protein, 5 aa, synthetic construct
DGGRH

SEQ ID NO: 133 - protein, 5 aa, synthetic construct
DGSRH

SEQ ID NO: 134 - protein, 5 aa, synthetic construct
NGGNH

SEQ ID NO: 135 - protein, 5 aa, synthetic construct
NGGHH

SEQ ID NO: 136 - protein, 5 aa, synthetic construct
DGSNH

SEQ ID NO: 137 - protein, 5 aa, synthetic construct
DGARH

SEQ ID NO: 138 - protein, 5 aa, synthetic construct
DGAQH

SEQ ID NO: 139 - protein, 5 aa, synthetic construct
DGAEH

SEQ ID NO: 140 - protein, 5 aa, synthetic construct
DGARN

SEQ ID NO: 141 - protein, 8 aa, synthetic construct
DINKFLWM

SEQ ID NO: 142 - protein, 8 aa, synthetic construct
DLMKSPHM

SEQ ID NO: 143 - protein, 8 aa, synthetic construct
DLNKPTPM

SEQ ID NO: 144 - protein, 8 aa, synthetic construct
MLRIMPVV

SEQ ID NO: 145 - protein, 8 aa, synthetic construct
MSLIFSTV

SEQ ID NO: 146 - protein, 8 aa, synthetic construct
MLRLGFWI

SEQ ID NO: 147 - protein, 8 aa, synthetic construct
NIRLAPWI

SEQ ID NO: 148 - protein, 8 aa, synthetic construct
NIRLSPWI

SEQ ID NO: 149 - protein, 8 aa, synthetic construct
NLRLAYWI

SEQ ID NO: 150 - protein, 8 aa, synthetic construct
NIRLAYWI

SEQ ID NO: 151 - protein, 8 aa, synthetic construct
DVRLAYWI

SEQ ID NO: 152 - protein, 8 aa, synthetic construct
DTNLAYWI

SEQ ID NO: 153 - protein, 8 aa, synthetic construct
QLRLGAWI

SEQ ID NO: 154 - protein, 8 aa, synthetic construct
HLRLGIWI

SEQ ID NO: 155 - protein, 8 aa, synthetic construct
LIPISHWI

SEQ ID NO: 156 - protein, 22 aa, synthetic construct
IIASVSEHQPTTWVSFFFDLHI

SEQ ID NO: 157 - protein, 22 aa, synthetic construct
IIASVSEHQPPTWPSYFMDINV

SEQ ID NO: 158 - protein, 22 aa, synthetic construct
IIASVSEHQPTAWSSFMFDYHI

SEQ ID NO: 159 - protein, 22 aa, synthetic construct
LVDSVAEHQPASPEAMWAFLHV

SEQ ID NO: 160 - protein, 22 aa, synthetic construct
LVDSVAEHRPTTAGAYLRYFHV

SEQ ID NO: 161 - protein, 22 aa, synthetic construct
VNETIMEVNTIDPEVFMQRISS

SEQ ID NO: 162 - protein, 22 aa, synthetic construct
VNQTIQEVENVDLSEFMRRISG

SEQ ID NO: 163 - protein, 22 aa, synthetic construct
VAQSIIEVQDLSLSEVLSYFHP

SEQ ID NO: 164 - protein, 22 aa, synthetic construct
IGQSVIEVQNIALERVLERFHT

SEQ ID NO: 165 - protein, 22 aa, synthetic construct
IGQSVIEAQNLPLAEVFDRFHP

SEQ ID NO: 166 - protein, 22 aa, synthetic construct
IAQSVIEAQNISINDLLTNLTG

SEQ ID NO: 167 - protein, 22 aa, synthetic construct
IAQSVIEAQNISFDALFANLTG

SEQ ID NO: 168 - protein, 22 aa, synthetic construct
IAQSVIEAQNLSFDVFFANLTG

SEQ ID NO: 169 - protein, 22 aa, synthetic construct
IGQSVIEVQNVKLVALLGDLTG

SEQ ID NO: 170 - protein, 22 aa, synthetic construct
IAQSVIEAQDVKFDDLFYNIMG

SEQ ID NO: 171 - protein, 22 aa, synthetic construct
IGQSVIEVQNVKLVALLSDLTG

SEQ ID NO: 172 - protein, 22 aa, synthetic construct
IAQSIIEAQNISFDALFANLTG

SEQ ID NO: 173 - protein, 22 aa, synthetic construct
IGQSVIEAQNLPLAEVFARFHP

SEQ ID NO: 174 - protein, 22 aa, synthetic construct
VAQSIIEVQDLSLAEVLAYFHP

SEQ ID NO: 175 - protein, 22 aa, synthetic construct
VVGSIIEAQKLSFDAFLTSVHG

SEQ ID NO: 176 - protein, 22 aa, synthetic construct
VAQSIIEVQDLSVAEVLAYFHP

SEQ ID NO: 177 - protein, 22 aa, synthetic construct
VAQSIIEVQEVNLDELLSYIYP

SEQ ID NO: 178 - protein, 22 aa, synthetic construct
VNQSIIEVQDLSFAALFPYFHP

SEQ ID NO: 179 - protein, 22 aa, synthetic construct
VAQSIIEVQDLSFAALFPYFHP

SEQ ID NO: 180 - protein, 22 aa, synthetic construct
VTQSIVEVHDIKFKELLTYLHP

SEQ ID NO: 181 - protein, 22 aa, synthetic construct
VAQSIIEVQDLSLSGLLPYFHP

SEQ ID NO: 182 - protein, 22 aa, synthetic construct
AGQALTEVQDLGLLAVLAYFHP

SEQ ID NO: 183 - protein, 47 aa, synthetic construct
DAPAKPQTPAVESPEANGTAANATQPTNGTESGENTTQPANTTQHHH

SEQ ID NO: 184 - protein, 10 aa, synthetic construct
ALEGGQYNST

SEQ ID NO: 185 - skipped

SEQ ID NO: 186 - protein, 24 aa, synthetic construct
VIHEFDPYFNYRTTRFLAEEGFYK

SEQ ID NO: 187 - protein, 24 aa, synthetic construct
IIHEFDPWFNYRSTHHLASHGFYE

SEQ ID NO: 188 - protein, 24 aa, synthetic construct
VIHEFDPYFNYRVTQFLSKNGIYE

SEQ ID NO: 189 - protein, 24 aa, synthetic construct
MIHEFDPYFNYRTTLFLTEKGFYE

SEQ ID NO: 190 - protein, 24 aa, synthetic construct
LIHEFDPWFNYRAAEYMSTHGWSA

SEQ ID NO: 191 - protein, 24 aa, synthetic construct
LIHEFDPWFNYRATQYLSDNGWRA

SEQ ID NO: 192 - protein, 30 aa, synthetic construct
CRLYWVAWASEFYEFFFNDQLMITTNDGYA

SEQ ID NO: 193 - protein, 30 aa, synthetic construct
CRFYWVWWASEFNEYFFNNQLMIISNDGYA

SEQ ID NO: 194 - protein, 27 aa, synthetic construct
MHEIYGWQNPALVVDGEFIQGTHDSYH

SEQ ID NO: 195 - protein, 27 aa, synthetic construct
MQELPEWNQPAYRVAGEFIMGTHDAYH

SEQ ID NO: 196 - protein, 27 aa, synthetic construct
MQELPSWNQEAYKVAGEFIMGTHDAYH

SEQ ID NO: 197 - skipped

SEQ ID NO: 198 - protein, 19 aa, synthetic construct
PGYISRSVAGSYDNEGIAI

SEQ ID NO: 199 - protein, 19 aa, synthetic construct
PGYISRSVAGSFDNEGIAI

SEQ ID NO: 200 - protein, 19 aa, synthetic construct
PSYISRSVAGSYDNEAVAI

SEQ ID NO: 201 - protein, 19 aa, synthetic construct
PGYISRSVAGSYDNEAVAI

SEQ ID NO: 202 - protein, 19 aa, synthetic construct
PAHLMRSMAGEFDNECIAV

SEQ ID NO: 203 - protein, 19 aa, synthetic construct
PAHLMRSMAGEFDNECVAM

SEQ ID NO: 204 - protein, 19 aa, synthetic construct
NSYYNRTMSGYYDTDMLVL

SEQ ID NO: 205 - protein, 19 aa, synthetic construct
NSYYNRTMSGYYDTDMLVI

SEQ ID NO: 206 - protein, 19 aa, synthetic construct
PGFYFRSRLGYYDTDMVTL

SEQ ID NO: 207 - protein, 19 aa, synthetic construct
PGYYYRSRLGYYDTDIVTL

SEQ ID NO: 208 - skipped

SEQ ID NO: 209 - protein, 31 aa, synthetic construct
KVMSWWDYGYQITAMANRTILVDNNTWNNTH

SEQ ID NO: 210 - protein, 31 aa, synthetic construct
RVMSWWDYGYQIAGMANRTTLVDNNTWNNSH

SEQ ID NO: 211 - protein, 31 aa, synthetic construct
KVASWWDYGYQTTAMANRTVIVDNNTWNNTH

SEQ ID NO: 212 - protein, 31 aa, synthetic construct
KIMSWWDYGYQITAMGNRTVIVDNNTWNNTH

SEQ ID NO: 213 - protein, 31 aa, synthetic construct
RVLAWWDYGYQITGIGNRTSLADGNTWNHEH

SEQ ID NO: 214 - protein, 31 aa, synthetic construct
RILSWWDYGYQITGIGNRTTLADGNTWSHKH

SEQ ID NO: 215 - protein, 31 aa, synthetic construct
YVVAWWDYGYPIRYYSDVKTLIDGGKHLGKD

SEQ ID NO: 216 - protein, 31 aa, synthetic construct
YVVTWWDYGYPVRYYSDVKTLVDGGKHLGKD

SEQ ID NO: 217 - protein, 31 aa, synthetic construct
KVWTWWDWGYPTHYYAERISFADGGRHYGHH

SEQ ID NO: 218 - protein, 31 aa, synthetic construct
EVWTWWDWGYATHYYAGLQSFADGGRHYGEH

SEQ ID NO: 219 - protein, 31 aa, synthetic construct
QVWTWWDWGYATHYYAQLHSFADGGRHYGEH

SEQ ID NO: 220 - protein, 11 aa, synthetic construct
SDDINKFLWMV

SEQ ID NO: 221 - protein, 11 aa, synthetic construct
GDDINKFLWMV

SEQ ID NO: 222 - protein, 11 aa, synthetic construct
SGDLMKSPHMA

SEQ ID NO: 223 - protein, 11 aa, synthetic construct
GGDLNKPTPMM

SEQ ID NO: 224 - protein, 27 aa, synthetic construct
RDVYIYMPYRMLRIMPVVAQFANTNPD

SEQ ID NO: 225 - protein, 27 aa, synthetic construct
RDIYLYMPARMSLIFSTVASFSFINLD

SEQ ID NO: 226 - protein, 19 aa, synthetic construct
APRYVVATFENIRLAPWIT

SEQ ID NO: 227 - protein, 19 aa, synthetic construct
MPLYVVATFENIRLSPWIC

SEQ ID NO: 228 - protein, 19 aa, synthetic construct
TSQYVVATFENIRLSPWIC

SEQ ID NO: 229 - protein, 22 aa, synthetic construct
IIASVSEHQPTTWSSYYFDLQL